(19) United States
(12) Patent Application Publication
(10) Pub. No.: US 2021/0401453 A1
(43) Pub. Date: Dec. 30, 2021

(54) SYSTEMS AND METHODS FOR SKIN TREATMENT

(71) Applicant: Cytrellis Biosystems, Inc., Woburn, MA (US)

(72) Inventors: Kristian DiMatteo, Waltham, MA (US); Samantha Higer, Boston, MA (US); Anna Vogel, Boston, MA (US); Robert Brik, Boston, MA (US); Alan Clark, Boston, MA (US); Duncan Silver, Boston, MA (US); Michail Pankratov, Boston, MA (US); Oivind Brockmeier, Boston, MA (US)

(21) Appl. No.: 17/291,235

(22) PCT Filed: Nov. 6, 2019
(86) PCT No.: PCT/US19/60131

§ 371 Date: May 4, 2021

Related U.S. Application Data
(60) Provisional application No. 62/756,694, filed on Nov. 7, 2018.

(51) Int. Cl. A61B 17/3205

(52) U.S. Cl. CPC A61B 17/32053; A61B 2017/00398; A61B 2217/005

(57) ABSTRACT

Described herein are technologies, methods, and/or devices for treating skin (e.g., eliminating tissue volume, tightening skin, lifting skin, and/or reducing skin laxity) by selectively excising a plurality of microcores without thermal energy being imparted to surrounding (e.g., non-excised) tissue. The technologies, methods, and/or devices described herein satisfy an unmet need for rapid and safe treatment of skin, including, e.g., faster pretreatment preparation and post-treatment healing times compared to current surgical and thermal treatment methods.

### RELATED APPLICATIONS

This application claims priority to and the benefit of U.S. Provisional Patent Application Ser. No. 62/756,694, filed Nov. 7, 2018, entitled “Systems and Methods for Skin Treatment,” the disclosure of which is incorporated herein by reference in its entirety.

### FIELD OF TECHNOLOGY

The technologies described herein generally relate to systems and methods for treatment of biological tissues.

### BACKGROUND

Many human health issues arise from damage, deterioration, or loss of tissue due to disease, advanced age, and/or injury. These health issues can manifest themselves in a variety of alterations of tissue structure and/or function, including scarring, sclerosis, tightness, and laxity. In aesthetic medicine, elimination of excess tissue and/or skin laxity is an important concern that affects more than 25% of the U.S. population. In a recent survey (September 2015) of 1052 women in the US (ages 35-75), 78% of women surveyed felt that they had sagging skin, and 83% of these women were self-conscious about it. In addition, 86% of women surveyed felt that they had wrinkles.

### SUMMARY

There is a need for improved systems and methods that provide increased effectiveness over currently available minimally-invasive techniques while maintaining convenience, affordability, and accessibility to patients desiring tissue restoration.

Described herein are technologies, methods, and/or devices for treating skin (e.g., eliminating tissue volume, tightening skin, lifting skin, and/or reducing skin laxity) by selectively excising a plurality of microcores without thermal energy being imparted to surrounding (e.g., non-excised) tissue. The technologies, methods, and/or devices described herein satisfy an unmet need for rapid and safe treatment of skin, including, e.g., faster pretreatment preparation and post-treatment healing times compared to current surgical and thermal treatment methods.

In some aspects, this disclosure provides an apparatus for producing a cosmetic effect in skin tissue. In some embodiments, a provided apparatus may include a needle hub comprising at least one hollow needle having a distal end for contacting skin and configured (e.g., having a microcore) to remove a portion of the skin tissue when the hollow needle is inserted into and withdrawn from the skin tissue. An apparatus may include a translation and/or actuation mechanism connected to a needle hub to translate and/or actuate the needle hub in one or more directions relative to a surface of the skin tissue. An apparatus may include a spacer to stabilize skin tissue and/or maintain a constant position of the apparatus relative to a surface of skin tissue.

In some embodiments, an apparatus may include a hand piece shell at least partially enclosing the translation and/or actuation mechanism. In some embodiments, a spacer may be attached to the hand piece shell.

In some embodiments, a needle hub may include a single hollow needle. In some embodiments, a needle hub may include three hollow needles arranged in a row. In some embodiments, a needle hub may include a two dimensional array of needles (e.g., a two-by-two, three-by-two, or three-by-three array).

In some embodiments, a needle hub may include a first lumen having a first end and a second end. A first lumen may be or may include a lumen of at least one hollow needle and the first end of the first lumen may be at a distal end of the hollow needle.

In some embodiments, a needle hub may include a second lumen having a wall, a first end, and a second end. A first end of the second lumen may be or may include a fluid intake nozzle. In some embodiments, a first lumen may be connected to a second lumen such that the second end of the first lumen forms an opening in the wall of the second lumen. In some embodiments, each of a first lumen and a second lumen may be substantially straight, and the first lumen may be substantially perpendicular to the second lumen forming a T-junction. In some embodiments, a fluid intake nozzle may be a convergent nozzle.

In some embodiments, a second end of a second lumen may be connected to a fluid conduit such that when low pressure or (partial) vacuum is applied to the conduit, low pressure or (partial) vacuum is induced in the first lumen and the second lumen, such that fluid is drawn into and through the second lumen through the first end of the second lumen, thereby clearing skin tissue from the first lumen.

In some embodiments, a provided apparatus may include a translation and/or actuation mechanism including an actuator to displace a needle hub along a z-axis in a direction substantially perpendicular to a surface of skin tissue and substantially parallel to a longitudinal axis of at least one hollow needle. In some embodiments, an actuator may be or include a voice coil. In some embodiments, an apparatus may include a sensing device for detecting a position of a needle hub along a z-axis. In some embodiments, a translation and/or actuation mechanism may include an x/y-stage to translate the needle hub in one or more directions parallel to the surface of the skin. In some embodiments, an apparatus may include a translation and/or actuation mechanism including a rotary stage to rotate the needle hub around the z-axis.

In some embodiments, a provided apparatus may include a spacer including a device to contact a surface of the skin tissue and to maintain a distance and/or position between the apparatus and the skin tissue. In some embodiments, a provided apparatus may include a spacer including a device to contact a surface of the skin tissue to maintain or increase tension in the skin tissue during treatment compared to skin tissue not being treated and/or contacted by an apparatus.

In some embodiments, a provided apparatus may include a spacer including a frame to contact a surface of a skin tissue, wherein the frame comprises a base, an inner wall, and an outer wall, wherein the base, inner wall, and outer wall form an open channel.

In some embodiments, a channel may be configured such that when a frame is placed on a surface of skin, the surface of the skin, the base, the inner wall, and outer wall form a frame lumen. In some embodiments, a frame may be connected to a fluid conduit such that when low pressure or (partial) vacuum is applied to the conduit, low pressure or (partial) vacuum is established in a frame lumen, thereby drawing skin tissue toward and/or into a channel. In some embodiments, a base may include one or more protrusions. In some embodiments, a frame may be contoured (e.g., wherein the frame is concave).

In some embodiments, a spacer may include a switch connected to a sensor to detect a position of an apparatus relative to tissue underlying skin. When a frame is in a first position, e.g., is placed on a surface of skin and a low pressure or (partial) vacuum is applied to the frame, the switch may be in a “no-go” position. When a frame is in a second position, e.g., while the frame is in contact with a surface of skin after a low pressure or (partial) vacuum is applied to the frame, and after the frame is moved in a direction that is substantially perpendicular to and away from the surface of the skin, the switch may be in a “go” position. When a switch is in the no-go position, a needle hub may be prevented from moving along a z-axis in a direction substantially perpendicular to a surface of the skin tissue and substantially parallel to a longitudinal axis of the at least one hollow needle. When a switch is in the go position, a needle hub may be moveable along the z-axis. In some embodiments, a sensor may be or include a pushrod.

In some aspects, this disclosure provides a system including an apparatus as described herein. In some embodiments, a provided system may include a removal system for removing one or more tissue portions from an apparatus. In some embodiments, a removal system may include a low pressure source (e.g., a vacuum pump). In some embodiments, a low pressure source may be connected to a needle hub including at least one hollow needle via a first conduit to provide suction in the at least one hollow needle. In some embodiments, a low pressure source is connected to a spacer via a second conduit to provide suction in the spacer.

In some embodiments, at least one hollow needle may include at least a first prong provided at a distal end of the hollow needle for contacting skin. In some embodiments, an angle between a lateral side of a first prong and a longitudinal axis of the hollow needle may be at least about 20 degrees. In some embodiments, at least one hollow needle may include a second prong at a distal end of the hollow needle. In some embodiments, a first prong and/or a second prong may include a flat tip. In some embodiments, a first prong and/or a second prong may include an edge. In some embodiments, an inner diameter of at least one hollow needle may be between about 0.14 mm and 0.84 mm. In some embodiments, an inner diameter of at least one hollow needle may be between about 0.24 mm and 0.40 mm.

In some embodiments, at least one hollow needle may be configured to extend (i) into the dermal layer, (ii) through the entire dermal layer to the junction of the dermal layer and the subcutaneous fat layer, or (iii) into the subcutaneous fat layer.

At least part of the processes and systems described in this specification may be controlled by executing, on one or more processing devices, instructions that are stored on one or more non-transitory machine-readable storage media. Examples of non-transitory machine-readable storage media include, but are not limited to, read only memory, an optical disk drive, memory disk drive, random access memory, and the like. At least part of the processes and systems described in this specification may be controlled using a computing system comprised of one or more processing devices and memory storing instructions that are executable by the one or more processing devices to perform various control operations.

The details of one or more implementations are set forth in the accompanying drawings and the description. Other features and advantages will be apparent from the description and drawings, and from the claims.

### BRIEF DESCRIPTIONS OF THE DRAWINGS

FIG. 1 is a cut-away view of an example apparatus for microcoring.

FIG. 2 is a cut-away view of an example apparatus for microcoring.

FIG. 3 is a perspective view of an example apparatus for microcoring.

FIG. 4 is a perspective cutaway view of an example apparatus for microcoring.

FIG. 5 is a perspective view of an example apparatus for microcoring.

FIG. 6 is a perspective view of an example actuation unit.

FIG. 7A and FIG. 7B are cut-away view diagrams of example needles inserted in skin tissue.

FIG. 8 shows an example plot of longitudinal displacement (z-axis displacement) of an example voice coil during a coring procedure against time, and electrical power (Voltage (V)×Current (I)×Voice coil constant (K)) consumed by the voice coil against time.

FIG. 9 shows an example histogram of number of coring strokes (e.g., a stroke being a single z-actuation cycle of a needle hub) against area of under a power curve (e.g., as shown in FIG. 8) as a measure of work done by a voice coil, e.g., of a z-actuator.

FIG. 10 shows an example plot of voice coil velocity, position, and acceleration against time during an example normal coring procedure.

FIG. 11 shows an example plot of voice coil velocity, position, and acceleration against time before, during, and after a coring procedure with excessive over penetration and contact with hard tissue.

FIG. 12 is a cut-away view diagram of an example needle mounted on an example needle hub inserted in skin tissue.

FIG. 13 is a side view diagram of two example apparatuses for microcoring having depth control spacer elements with different lengths.

FIG. 14 is a side view of three different example needle hub configurations with needles of different lengths.

FIG. 15 is a side view diagram of an example embodiment of an example apparatus for microcoring where a depth control spacer element is threaded onto a tubular region at a distal end of the example apparatus.

FIG. 16 is a cut-away view diagram of an example apparatus with an example mechanism, e.g., to raise or lower (relative to a skin surface during operation) a z-actuator.

FIG. 17 shows an example coring pattern for coring using an example apparatus for microcoring.

FIG. 18 shows an example coring pattern for coring using an example apparatus for microcoring.

FIG. 19 is a semi-transparent cut-away view diagram of a retractable “pen-click”-type rotary mechanism for an example apparatus for microcoring.

FIG. 20A is a cut-away views of an example needle hub with one coring needle. FIG. 20B is a semi-transparent view of an example needle hub with one coring needle. FIG. 20C is a diagram illustrating an example core clearing procedure in an example needle hub with one coring needle.

FIG. 21 shows example results of a computational fluid dynamics simulation of fluid flow in an example channel of an example needle hub. Arrows indicate flow direction. Gray scale of arrows indicates Mach number.

FIG. 22 shows example results of a computational fluid dynamics simulation of fluid flow in an example channel of an example needle hub. Gray scale indicates fluid pressure.

FIG. 23A and FIG. 23B are cross-sectional view diagrams of an example needle and with lateral openings.

FIG. 24A, FIG. 24B, and FIG. 24C are cross-sectional view diagrams of components of an example fluid-based system for removal of tissue from an example needle.

FIG. 25A, FIG. 25B, and FIG. 26C are cross-sectional view diagrams of components of an example membrane-based system for removal of tissue from an example needle.

FIG. 26A, FIG. 26B, FIG. 26C, and FIG. 26D are cross-sectional view diagrams of components an example pushrod and membrane based system for removal of tissue from a needle.

FIG. 27 is a perspective exploded view of components of an example needle hub and core clearing system with three needles.

FIG. 28A and FIG. 28B, are cross-sectional views of an example needle hub for three needles. FIG. 28C, FIG. 28D and FIG. 28E are perspective views of an example needle hub for three needles.

FIG. 29A is a cross-sectional view of an example needle hub insert. FIG. 29B and FIG. 29C are perspective views of an example needle hub insert.

FIG. 30 is a perspective view of components of an example needle hub and core clearing system with three needles.

FIG. 31 is a semi-transparent cut-away view of an example needle hub with three needles.

FIG. 32A is a perspective view of an example needle hub with an example hub shield.

FIG. 32B is a cross-sectional view of an example needle hub with an example hub shield. FIG. 32C is a side view of an example needle hub with an example hub shield.

FIG. 33 is a perspective view of an example needle hub with an example hub shield and an example spacer.

FIG. 34 is a cross-sectional view of an example needle hub assembly including an ingress shield on the needle hub.

FIG. 35 is a cross-sectional schematic diagram view of an example needle hub assembly including a cylindrical ingress shield on the needle hub.

FIG. 36 is a perspective exploded view of components of an example needle hub and core clearing system with one needle.

FIG. 37A is a cross-sectional view of an example needle hub for one needle. FIG. 37B is an enlargement of the encircled portion in FIG. 37A. FIG. 37C, FIG. 37D and FIG. 37E are perspective views of an example needle hub for one needle.

FIG. 38A, FIG. 38B, and FIG. 38C show example needle hubs with one, two, and three needles, respectively.

FIG. 39A, FIG. 39B, and FIG. 39C show example needle array configurations.

FIG. 40A, FIG. 40B, and FIG. 40C are perspective views of an example vacuum spacer.

FIG. 41A, and FIG. 41B, are perspective views of an example vacuum spacer.

FIG. 42A is a cross-sectional view of an example vacuum spacer. FIG. 42B is a partial cross-sectional view of an example vacuum spacer.

FIG. 43 is a perspective view of an example vacuum spacer system.

FIG. 44 is a perspective view of an example vacuum spacer frame.

FIG. 45 is a perspective view of a schematic of an example curved vacuum spacer frame.

FIG. 46 is a perspective view of a schematic of an example vacuum spacer frame.

FIG. 47 is a perspective view of a schematic of an example vacuum spacer frame grid.

FIG. 48 is a perspective cross-sectional view of a schematic of an example vacuum spacer frame element.

FIG. 49 is a perspective view of an example vacuum spacer frame including an example pressure foot system.

FIG. 50 is a perspective view of an example vacuum spacer frame including an example pressure foot system.

FIG. 51 is a perspective view of an example vacuum spacer frame including an example pressure foot system.

FIG. 52 is a diagram of an example low pressure or (partial) vacuum system.

FIG. 53 is a diagram of an example low pressure or (partial) vacuum system with a subsystem for a needle hub and a subsystem for a vacuum spacer.

FIG. 54 is a perspective view of an example vacuum alignment frame including a low pressure or (partial) vacuum channel and protrusions on an inner wall.

FIG. 55 is a perspective view of an example distal end component of an example apparatus for microcoring.

FIG. 56 is a perspective view of an example distal end component of an example apparatus for microcoring including a vacuum frame.

FIG. 57A and FIG. 57B are side views of a spacer and frame with a moveable alignment element.

FIG. 58A is a perspective view of an example apparatus for microcoring with a spacer including a frame including an inner alignment element in form of a sub-frame. FIG. 58B shows an example spacer frame including an inner alignment element in form of a sub-frame.

FIG. 59A shows an example spacer frame including an inner alignment element in form of a grid of wires. FIG. 59B shows an example spacer frame including an inner alignment element in form of a transparent element.

FIG. 60 is a side view of an example apparatus for microcoring with spacer frame including a mirror assembly.

FIG. 61 is a perspective view of an example apparatus for microcoring with spacer including a camera.

FIG. 62 is a perspective view of an example apparatus for microcoring with spacer including a light source.

FIG. 63 is a perspective view of an example vision system to provide a display of a treatment region.

FIG. 64 shows an example output of an image processing system indicating complete core removal (circles) and incomplete core removal (no circle).

FIG. 65 shows a diagram of motion tracking of an example apparatus for microcoring across a skin surface.

FIG. 66 is a cross-sectional view of a section of an example apparatus for microcoring including an ingress shield.

FIG. 67 is a perspective view of an example ingress shield for an apparatus for microcoring.

FIG. 68 is a cross-sectional view diagram of an example of an ingress shield for an apparatus for microcoring.

FIG. 69 is a cross-sectional view diagram of an example sliding plate for protection of an apparatus for microcoring.

FIG. 70 is a perspective cut-away view of an apparatus for microcoring including a needle hub mount.

FIG. 71A is a side view diagram of an example apparatus for microcoring with an array of (disposable) needle hubs. FIG. 71B is a side view diagram of an example apparatus for microcoring with an array of (disposable) spacers.

FIG. 72 is a side view diagram of an example apparatus for microcoring with an array of (disposable) needle hubs and (disposable) spacers.

FIG. 73 shows an example needle hub attachment and/or replacement system and procedure of an example apparatus for microcoring.

FIG. 74 is a perspective view of an example spacer including channels and a distal section of a hand piece of an example apparatus for microcoring including rails for connection to the spacer.

FIG. 75 is a cross-sectional view of an example spacer and distal section of a hand piece of an example apparatus for microcoring with a snap on/pinch off connection mechanism.

FIG. 76 is a perspective view of an example spacer having a thread for connection to an example apparatus for microcoring.

FIG. 77A-77D shows an example spacer mounting system to connect a needle hub and/or a spacer to an example apparatus for microcoring.

FIGS. 78A and 78B is a cross-sectional view diagram of an example adhesive adhesive film to remove microcores from skin tissue.

FIGS. 79A and 79B is a cross-sectional view diagram of an example suction device to remove microcores from skin tissue.

FIG. 80 is a cross-sectional view diagram of a scraping device to remove microcores from skin tissue.

FIG. 81 is a perspective view of possible needle prong configurations for a hollow needle.

FIG. 82 is a schematic illustration showing a side view of a prong of a hollow needle. A bevel angle α of a prong refers to the angle between lateral side of the prong and longitudinal axis of the hollow needle.

FIG. 83 shows photographs that compare needle heel degradations after 2,000, 8,000, and 10,000 actuation cycles of hollow needles having a bevel angle of 10 degrees, 20 degrees, or 30 degrees.

FIGS. 84A, 84B, 84C, and 84D are photographs showing experimental results indicating that hollow needles coated with diamond-like carbon (DLC) did not display any sign of needle heel degradation after 10,000 actuation cycles, while non-coated hollow needle showed needle heel degradation after 10,000 actuation cycles. FIG. 84A is a photograph of a DLC-coated needle before undergoing any actuation cycles; FIG. 84B is a photograph of the DLC-coated needle after undergoing 5,000 actuation cycles; FIG. 84C is a photograph of the DLC coated needle after undergoing 10,000 actuation cycles; and FIG. 84D is a photograph of a non-coated needle after undergoing 10,000 actuation cycles.

FIG. 85 a schematic illustration showing needle coring force and tissue resistance force on a cored tissue portion inside the lumen of an example hollow needle.

### DETAILED DESCRIPTION OF CERTAIN EMBODIMENTS

Described herein are technologies, methods, and/or devices that may be used, for example, for treating skin (e.g., eliminating tissue volume, tightening skin, lifting skin, and/or reducing skin laxity) by selectively excising a plurality of microcores without thermal energy being imparted to the surrounding (e.g., non-excised) tissue.

Microcoring

In general, the term “microcoring,” as used herein, refers to technologies that utilize one or more (in some embodiments, a plurality, e.g., an array) hollow needles, or other non-thermal implement(s) (e.g., blades, tubes, or drills), of sufficiently small dimension to minimize the extent of bleeding and/or clotting within holes or slits, and/or to minimize scar formation, to excise and optionally sequester tissue from a site. In some embodiments, excising a tissue means forming a tissue portion (e.g., a “microcore”), e.g., by inserting a hollow needle into the site so that the tissue portion is formed inside the hollow needle and severed from surrounding tissue, whereby a microcore that is separate from other tissue is generated.

In some embodiments, microcoring technologies as described herein may include sequestration of the excised tissue. As used herein, the term “sequestering”, when used in reference to tissue, means excising a microcore and then removing the excised microcore from the excision site. In certain embodiments, sequestered tissue may be permanently disposed. In certain embodiments, sequestered tissue may be used for diagnostic purpose, e.g., using biopsy and/or histology techniques known in the art. In many embodiments, technologies provided herein maximize removal and minimize risk of (partial or complete) re-insertion of extracted tissue.

It should be understood that particular microcoring technologies, methods, and/or devices using hollow needles specifically described herein serve for exemplary and/or illustrative purposes, and that other techniques and devices can be used to create microcores. Microcoring technologies described herein may include a number of advantageous features. For example, provided technologies may enable visualization of results in real time during the course of the treatment, e.g., through subject feedback and subsequent treatment adjustment in real time.

Alternatively or additionally, apparatuses used for microcoring can include micro-sized features that may be beneficial for controlling extent of skin treatment.

Still further, in some embodiments, methods and/or devices described herein may require less skill than that of a surgeon. Thus, in certain embodiments, a subject may be treated by a non-physician professional and/or in an outpatient setting, rather than in an inpatient, surgical setting. In some embodiments, a subject may be treated at a spa, at a cosmetic salon, or at home. That is, the present disclosure provides technologies that are amenable to and/or permit consistent and/or reproducible administration of skin treatment services.

In some embodiments, technologies, methods, and/or devices described herein may have generally a lower risk profile and can provide more predictable results and/or risk factors than those for more invasive techniques (e.g., plastic surgery) or energy-based techniques (e.g., laser, radiofrequency (RF), or ultrasound), which may or may not be invasive.

In some embodiments, non-thermal fractional excision technologies, methods, and/or devices described herein allow skin tightening, skin lifting, and/or reduction of skin laxity without (or with significant reduction of) one or more common side effects of thermal ablation methods. Thermal ablation techniques prevent and/or inhibit skin tightening by allowing coagulation of tissue and formation of rigid tissue cores that cannot be compressed. Thermal ablation techniques create a three-dimensional heat-affected zone (HAZ) surrounding an immediate treatment site. While fractional ablative lasers can be used on or near heat-sensitive sites (e.g., eyes, nerves), i.e., when the laser does not penetrate more than 1 mm into the skin (resulting in a comparatively small HAZ), other thermal ablation techniques (e.g., ultrasound based techniques) cannot be used in the vicinity of heat-sensitive sites because the HAZ may extend to heat sensitive tissues potentially causing (permanent) damage. As will be appreciated by those skilled in the art reading the present disclosure, a “heat-sensitive site” is a site where exposure to radiation and/or elevated temperature is associated with a relatively high risk of unacceptable cosmetic and/or physiologic outcomes. In any event, technologies, methods, and/or devices described herein have generally a lower risk profile than, e.g., thermal methods at least in part due to a zone of tissue injury that is smaller than the zone of injury (e.g., the HAZ) of thermal methods.

In some embodiments, advantages of certain technologies, methods, and/or devices described herein include a particularly low (e.g., lesser than that observed with other techniques such as invasive techniques and/or thermal techniques) degree of erythema, faster resolution of erythema, and lower percent incidence, severity, term of skin discoloration (hyperpigmentation or hypopigmentation), and/or particularly low swelling and/or inflammation, e.g., as compared, with that observed with laser treatment and/or with ultrasound-based treatment.

In some embodiments, certain technologies, methods, and/or devices provided herein can allow for rapid closing of holes or slits after excising tissue (e.g., within a few seconds after treating skin, such as within ten seconds), thereby minimizing extent of bleeding and/or clotting within holes or slits, and/or scar formation.

In some embodiments, certain technologies, methods, and/or devices provided herein may be useful for maximizing treatment effect while minimizing treatment time, e.g., by using rapid-fire reciprocating needles or needle arrays, and/or by using large needle arrays that allow for simultaneous excision of tens, hundreds, or even thousands of microcores.

In some embodiments, technologies, methods, and/or devices described herein may be useful for maximizing tightening effect while minimizing healing time and/or minimizing the time in which a cosmetic effect occurs by optimizing tightening (e.g., by controlling the extent of skin pleating, such as by increasing the extent of skin pleating for some applications or skin regions and by decreasing the extent of skin pleating for other applications or skin regions, as described herein).

In some embodiments, technologies, methods, and/or devices described herein may provide efficient clearance of sequestered or partially ablated tissue and/or debris from ablated tissue portions, thus reducing time for healing and improving the skin tightening treatment, e.g., relative to laser-based technologies.

In some embodiments, technologies, methods, and/or devices described herein may allow for efficient and effective positioning of skin prior to, during, and after excision and/or tissue sequestration. Positioning the skin is critical to control skin-tightening direction and ensure ablation occurs in the desired location and desired dimensions (e.g. thickness, width in a preferred direction, e.g., along or orthogonal to Langer lines).

Among other things, the present disclosure encompasses the insight that microcoring technologies may be developed (e.g., as described herein) that can achieve desirable procedure times and/or can significantly improve one or more aspects of healing from a procedure (e.g., a tissue removal procedure), compared to, e.g., thermal methods.

Systems and Articles for Microcoring

Overview

Described herein are technologies, methods, and/or devices for treating skin, e.g., by selectively micrcocoring skin tissue. In particular, described herein are hollow needles, as well as related apparatuses, kits, and methods, capable of microcoring tissue portions by capturing and retaining the tissue portions inside a lumen of one or more hollow needles after insertion into and withdrawal from the skin. Microcored tissue portions can be removed from a lumen of a hollow needle and discarded. The process can be repeated to generate multiple cored skin tissue portions, in particular over a desired area of skin and located at chosen sites of the body of a subject. The hollow needles, actuation units, apparatuses, kits, and methods described herein may provide increased effectiveness over currently available apparatuses and techniques while maintaining convenience, affordability, and accessibility to patients desiring tissue restoration.

In some embodiments, technologies described herein include an apparatus, e.g., a hand held apparatus. An example apparatus may include a needle hub comprising at least one hollow needle configured to remove a portion of the skin tissue (e.g., a microcore) when the hollow needle is inserted into and withdrawn from the skin tissue. In some embodiments, an apparatus may include a translation and/or actuation unit connected to the needle hub, e.g., to translate and/or actuate the needle hub in one or more directions relative to a surface of the skin tissue. In some embodiments, an apparatus may include a spacer to stabilize and/or maintain a constant position of the apparatus relative to the surface of the skin tissue. In some embodiments, an apparatus may include a hand piece including a hand piece shell, e.g., to at least partially encase the translation and/or actuation unit. In some embodiments, a hand piece and/or hand piece shell may include or may be connected to a spacer, e.g., at a distal end of an apparatus (e.g., an end of an apparatus for contacting skin).

Translation, Actuation and (Position) Detection

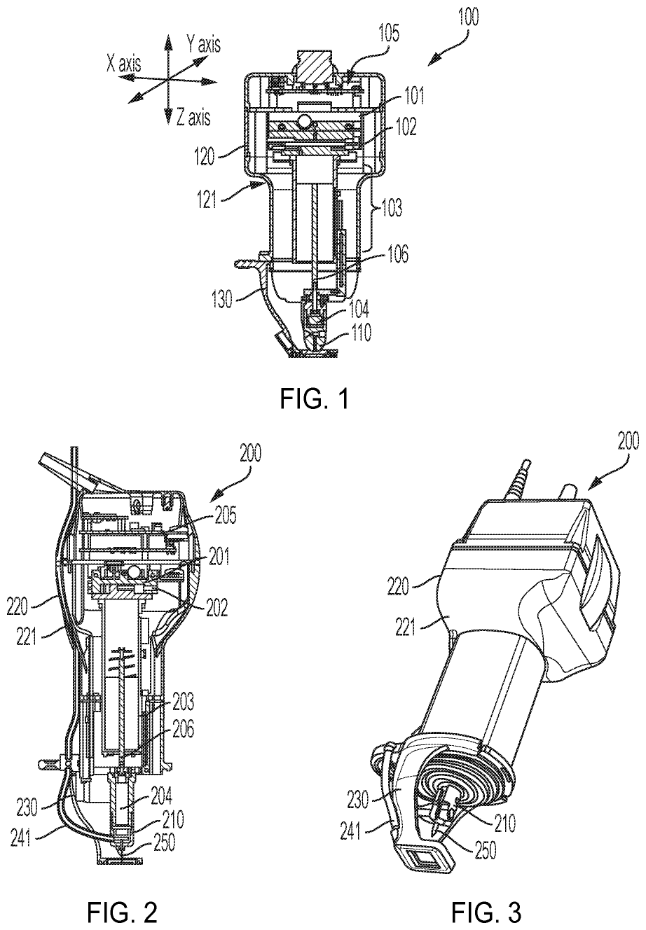

The technologies described herein may include a system that includes an apparatus for microcoring. An example apparatus 100 is shown in FIG. 1. An apparatus 100 as described herein may include an actuation unit including one or more actuation mechanisms to drive a needle hub and/or a hollow needle into skin (e.g., in a z-direction) or across skin (e.g., in an x- and/or y-direction). In some embodiments, an actuation unit of the apparatus 100 may be or include one or more x-actuators (e.g., x-actuator 101), one or more y-actuators (e.g., y-actuator 102), and one or more z-actuators (e.g., z-actuator 103). In some embodiments, an actuation mechanism, e.g., z-actuator 103, may be connected to a needle hub mount (e.g., needle hub mount 104) for removeably mounting a needle hub (e.g., needle hub 110) connected to one or more needles (not shown), e.g., via pushrod 106. In some embodiments, an apparatus for microcoring as described herein may be configured as a hand-held device that may be or include a hand piece (e.g., hand piece 120), e.g., comprising a hand piece shell (e.g., hand piece shell 121) encasing one or more components of an apparatus, e.g., actuators 101, 102, and/or 103, and/or other components, e.g., printed circuit board (PCB) 105, e.g., to control one or more actuators. A hand piece may include or may be removeably connected to other components of an apparatus 100, e.g., a spacer (e.g., spacer 130).

An example apparatus 200 is shown in FIG. 2. Apparatus 200 includes an x-actuator 201, a y-actuator 202, and z-actuator 203. Z-actuator 203 may be connected to a needle hub mount 204 for removeably mounting a needle hub 210 including an example needle 250, e.g., via pushrod 206. An example apparatus 200 may include a hand piece (e.g., hand piece 220), e.g., comprising a hand piece shell 221 encasing one or more components of an apparatus, e.g., actuators 201, 202, and 203, and/or other components, e.g., printed circuit board (PCB) 205, e.g., to control one or more actuators. A hand piece 220 may be removeably connected to one or more components of a system, e.g., a spacer 230. The example system may comprise a low pressure or (partial) vacuum system including vacuum tubing 241 connected to a needle hub 210. FIG. 3 shows an external view of apparatus 200.

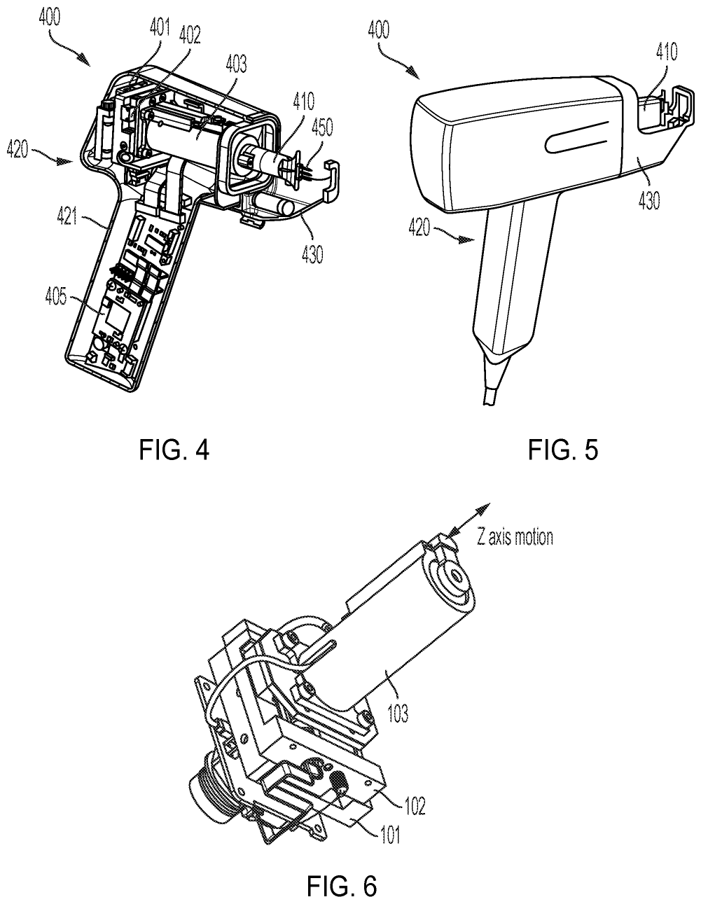

An example apparatus 400 is shown in FIG. 4. Apparatus 400 includes an x-actuator 401, a y-actuator 402, and z-actuator 403. Z-actuator 403 may be connected to a needle hub mount (not shown) for removeably mounting a needle hub 410 including one or more, e.g., three, example needles 450, e.g., via a pushrod (not shown). An example apparatus 400 may include a hand piece (e.g., hand piece 420), e.g., comprising a hand piece shell 421 encasing one or more components of an apparatus, e.g., actuators 401, 402, and 403, and/or other components, e.g., printed circuit board (PCB) 405, e.g., to control one or more actuators. A hand piece 420 may be removeably connected to one or more components of a system, e.g., a spacer 430. The example system may comprise a vacuum system including vacuum tubing (not shown) connected to a needle hub 410. FIG. 5 shows an external view of apparatus 400. Apparatuses 100, 200, and 400 are non-limiting example embodiments of technologies described herein. One or more features or components of apparatuses 100, 200, and 400 may be or may be used interchangeably.

In some embodiments, an actuation unit of the apparatus (e.g., an example actuation unit shown in FIG. 6) may include only x- and y-actuators (e.g., x-actuator 101, y-actuator 102) and/or a z-actuator 103. A z-actuator 103 (e.g., a voice coil, a solenoid, and/or a linear screw drive, disposed in z-axis housing) may be part of a needle assembly of the apparatus (e.g., a z-actuator and a needle hub). In some embodiments, x-, y-, and z-actuators may drive a needle hub and/or one or more hollow needles into and/or across a large area of skin surface in a relatively short amount of time compared to manual deployment of a hollow needle. In some embodiments, x-, y-, and z-actuators may drive a needle hub and/or one or more hollow needles into and/or across a small area of skin surface (e.g., a small area on the face (e.g., the area between the nose and the upper lip). In some embodiments, the x-, y-, and z-actuators may drive a needle hub and/or one or more hollow needle into and/or across multiple large and/or small areas of skin surface.

An example actuation unit as shown in FIG. 6 may include a z-actuator, e.g., a voice coil actuator, an x-actuator, e.g., an x-actuator stage comprising a linear screw drive, and a y-actuator, e.g., a y-actuator stage comprising a linear screw drive. In some embodiments, an x-actuator and a y-actuator have the same drive mechanism. In some embodiments, an x-actuator and a y-actuator have different drive mechanism. One or more actuators may be connected to a printed circuit board (e.g., as part of a control system), which may drive and/or control the actuators and/or receive feedback from the actuators, e.g., provide closed-loop control of actuation (e.g., to a control system). In some embodiments, an x-actuator and a y-actuator, e.g., an x-actuator stage and a y-actuator stage, may be stacked, e.g., forming an x/y-stage. In some embodiments, a z-actuator may be mounted on a stack of an x-actuator and a z-actuator, e.g., an x/y-stage, e.g. a z-actuator may be mounted on an x-actuator, and the x-actuator may be mounted on a y-actuator. In some embodiments, a stack of an x-actuator and a y-actuator may be mounted in or on a hand piece shell. In some embodiments, an x-actuator and a y-actuator may be mounted separately in or on a hand piece shell, e.g. the z-actuator is mounted and/or connected on an x-actuator and a y-actuator, e.g., on moveable tracks.

Z-Actuation

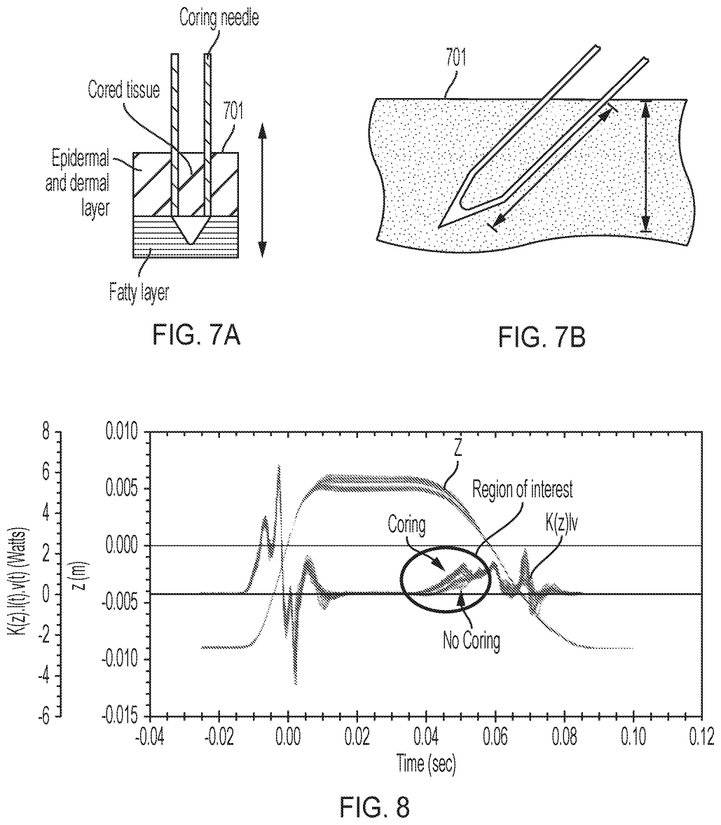

A z-actuator, e.g., z-actuator 103, 203, or 403, may drive displacement of a needle hub and/or one or more hollow needles along an axis (e.g., a z-axis), e.g., drive penetration into the skin by a hollow needle and/or retraction of the hollow needle after insertion (see, e.g., FIG. 7A). In some embodiments, a z-axis is substantially perpendicular to a skin surface 701 to be treated. In some embodiments, a z-axis is at an angle to a skin surface to be treated, e.g., about 90 degrees, 80 degrees, 70 degrees, 60 degrees, 50 degrees, 40 degrees, 30 degrees, 20 degrees, or 10 degrees. In some example embodiments, coring at an angle other than substantially perpendicular to a surface of skin may increase size of a microcore, and/or a ratio of dermis/epidermis to fat (see, e.g., FIG. 7B).

In some embodiments, a z-actuator, e.g., z-actuator 103, 203 or 403, may be located inside a hand piece (e.g., hand piece 120, 220, or 420), e.g., encased by a hand piece shell (e.g., hand piece shell 121. 221, or 421). In some embodiments, a z-actuator may be located external to a hand piece shell, wherein the z-actuator is mechanically coupled to a needle hub and/or one or more hollow needles.

In some embodiments, a z-actuator may be connected to a needle hub through a mounting assembly. In some embodiments, a mounting assembly may include a pushrod (e.g., a z-axis pushrod connected to a voice coil actuator, e.g., pushrod 106, 206, or 406) and a needle hub mount (e.g., needle hub mount 104, 204, or 404). In some embodiments, a z-actuator (e.g., a voice coil actuator) is part of a needle assembly of an apparatus and may be detachably attached to a needle hub.

A z-actuator as described herein may be capable of operating at a high speed to minimize treatment time and deflection of skin tissue during the penetration of the hollow needle. In some embodiments, one actuation cycle in the z-direction may take from about 5 milliseconds to about 50 milliseconds (e.g., 5, 10, 15, 20, 25, 30, 35, 40, 45, or 50 milliseconds). In some embodiments, a z-actuator may take about 20 to about 35 milliseconds (e.g., 20, 21, 22, 23, 24, 25, 26, 27, 28, 29, 30, 31, 32, 33, 34, or 35 milliseconds) to travel about 20 mm to about 30 mm (e.g., 20, 21, 22, 23, 24, 25, 26, 27, 28, 29, or 30 mm) distally toward and/or into skin tissue. In some embodiments, a z-actuator may take about 25 milliseconds to about 30 milliseconds (e.g., 25, 26, 27, 28, 29, or 30 milliseconds) to travel about 23 mm distally toward and/or into skin tissue. In some embodiments, a z-actuator may take about 25 to about 35 milliseconds (e.g., 25, 26, 27, 28, 29, 30, 31, 32, 33, 34, or 35 milliseconds (e.g., 30 milliseconds)) to travel about 20 mm to about 30 mm (e.g., 20, 21, 22, 23, 24, 25, 26, 27, 28, 29, or 30 mm (e.g., 23 mm)) proximally from a penetration depth of about 20 mm to about 30 mm (e.g., 20, 21, 22, 23, 24, 25, 26, 27, 28, 29, or 30 mm (e.g., 23 mm)) into skin tissue. In some embodiments, a z-actuator may take about 30 milliseconds to travel about 23 mm proximally from a penetrated skin tissue.

A z-actuator as described herein may further be capable of operating with a certain insertion force and/or retraction force. In some embodiments, a force of about 0.5 N to about 20 N (e.g., 0.5 N to 0.75 N, 0.5 N to 1 N, 0.5 N to 1.25 N, 0.5 N to 1.5 N, 0.5 N to 2 N, 0.5 N to 5 N, 0.5 N to 10 N, 0.5 N to 12 N, 0.5 N to 15 N, 0.5 N to 20 N, 0.75 N to 1 N, 0.75 N to 1.25 N, 0.75 N to 1.5 N, 0.75 N to 2 N, 0.75 N to 5 N, 0.75 N to 10 N, 0.75 N to 12 N, 0.75 N to 15 N, 0.75 N to 20 N, 1 N to 1.25 N, 1 N to 1.5 N, 1 N to 2 N, 1 N to 5 N, 1 N to 10N, 1 N to 12 N, 1 N to 15 N, 1 N to 20 N, 1.25 N to 1.5 N, 1.25 N to 2 N, 1.25 N to 5 N, 1.25 N to 10N, 1.25 N to 12 N, 1.25 N to 15 N, 1.25 N to 20 N, 1.5 N to 2 N, 1.5 N to 5 N, 1.5 N to 10N, 1.5 N to 12N, 1.5 N to 15N, 1.5 N to 20 N, 2 N to 5 N, 2 N to 10 N, 2 N to 12 N, 2 N to 15 N, 2 N to 20 N, 5 N to 10 N, 5 N to 12 N, 5 N to 15 N, 5 N to 20 N, 10 N to 12N, 10 N to 15 N, 10 N to 20 N, 12 N to 15 N, 12 N to 20 N, or 15 N to 20 N) per hollow needle may be applied, e.g., to ensure insertion of one or more hollow needles into skin. In some embodiments, a force of about 10 N to 20 N (e.g., 15 N) per hollow needle may be applied, e.g., to ensure insertion of one or more hollow needles into the skin. Without wishing to be bound by theory, insertion force may be inversely correlated with needle gauge. For example, a 24 gauge needle (e.g., a needle with an outer diameter of about 0.565 mm) may be operated with an insertion force of 12 N, while a 20 gauge needle (e.g., a needle with an outer diameter of about 0.9081 mm) may be operated with a higher insertion force. In some embodiments, an apparatus may include a feature or setting that may be used to control or change insertion force and/or retraction force of a hollow needle into/out of skin. In some embodiments, an adjustment implement, e.g., a scroll wheel, e.g., on a user interface of the a base unit (e.g., a unit comprising at least a part of a control system), may be used to adjust an insertion force and/or retraction force by the a hollow needle by physically adjusting (e.g., retracting) the hollow needle (e.g., adjusting position of a hollow needle relative to a distal end of an apparatus, e.g., when a z-actuator is fully retracted, e.g., by adjusting a position of a stationary base component of a z-actuator). In some embodiments, an adjustment implement, e.g., a scroll wheel on a user interface of a base unit, may be used to provide an electrical signal to a z-actuator to control insertion and/or retraction force. In some embodiments, a digital control unit or control system including a user interface of a base unit may control, e.g., distance, velocity, force and/or timing of penetration into and retraction out of the skin by a hollow needle. Parameters, e.g., insertion force as well as retraction force, may be monitored. In some embodiments, a z-actuator is or comprises a voice coil that includes and/or is connected to a closed loop position and/or momentum/energy control system, as described below.

In some embodiments, a z-actuator may provide position, velocity, acceleration, voice coil current, and/or voltage feedback signal to a z-axis position controller, e.g., a z-axis position controller that is part of a digital control unit as described herein. Feedback signals may be obtained from one or more sensors mounted on or integrated into a z-actuator. Feedback signals may be obtained from direct measurements, e.g., measurements of electric current and/or voltage entering or exiting a z-actuator, e.g., a voice coil. From these feedback signals, alone or in combination with known data, e.g., mass of a voice coil and/or needle assembly, a z-axis position controller (e.g., implemented on or as part of a digital control unit) may be used to measure and/or calculate a force required to insert/penetrate a subject's dermis and/or the force required to withdraw one or more coring needles from a subject's dermis.

A force required to penetrate dermal tissue may vary significantly between species, and may vary between subjects and/or skin types or areas to be treated. For example, abdominal dermal tissue may be thicker and/or tougher (harder to penetrate) than facial skin. Pig skin may be significantly thicker and/or tougher than human skin. A force required to penetrate dermal tissue may vary depending on number and/or configuration of needles used. Without wishing to be bound by theory, as the number of needles on a single needle hub increases, a force required to penetrate the dermis, e.g. full thickness dermis, may increase proportionately. An amount of force or energy required to fully penetrate a subject's dermis may be measured and may provide an in-vivo indication of a patient's skin toughness, and/or an indication of the resilience provided by the skin pressing against a coring needle in direction of the z-axis. This information may be useful to evaluate skin characteristics of a subject, e.g., skin laxity. Lax dermal tissue may provide less resistance to a penetrating needle as compared to healthy and/or firm skin. A measurement of a force or energy required to penetrate a subject's dermal layer may provide useful diagnostic information to a clinician. For example, as a subject's skin quality improves with each coring treatment, a specific increase in skin toughness (increased resilience provided by subject's skin against a penetrating coring needle) may be monitored from treatment to treatment, providing an indication of improvement in skin quality.

In some embodiments, a number of electrical and/or mechanical parameters of a system may be monitored before, during, and/or after coring, e.g., to determine tissue properties. Tissue properties may be inferred based on data from one or more sensors and/or data from electrical and/or mechanical parameters of a z-actuator entering and/or exiting tissue, e.g., voice coil (actuator) kinematics. Data may be used to characterize depth of tissue layers (e.g., dermis, epidermis, and/or fat), tissue quality of each layer (e.g., healthy, scarred, lax), and/or characterize location, shape, and/or volume of tissue features, e.g., scars or tumors, e.g., by combining said data with information of location (e.g., in an x-y plane of a treatment area) for each z-actuation.

In some embodiments, a coring process may be monitored to ensure successful coring and/or clearing of skin tissue from one or more hollow coring needles. In some embodiments, a measurement of force required to remove the coring needle from the patient's dermal layer may be used to indicate whether a core has been successfully withdrawn/excised or not. A force required to retract one or more needles with a (new) core present in a lumen of one or more needles may be different from a force required to retract one or more needles without a (new) core present in a lumen of one or more needles. In some embodiments, a digital control unit may be used to monitor data received from a voice coil of a z-actuator (e.g., position, velocity, and/or acceleration of a voice coil), current draw, counter-electromotive force (back EMF) and/or voltage, e.g., to derive successful coring information from voice coil data based on variation of force required to retract one or more needles from a tissue and/or variation of a velocity of needles being retracted from a tissue. In some embodiments, a radiofrequency energy may be applied to a needle, and output parameters may be monitored. Output parameters may vary based on presence of one or more cores inside a needle, thus indicating successful or unsuccessful coring. In some embodiments, radiofrequency energy may be applied to a needle to transfer energy to tissue, e.g., to improve coring and/or to core tissue selectively, e.g., by imparting a radiofrequency pulse when a needle is in contact with fat or septae. In some embodiments, heat may be generated in a tissue, e.g., through transfer of radiofrequency energy.

In some embodiments, amount and/or variation of pressure and/or flow rate in a fluid system in communication with one or more hollow needles may be monitored, e.g., using one or more pressure gauges, to determine successful coring. In some embodiments, successful coring may be verified using visual inspection of one or more components of a fluid system in communication with one or more needles, e.g., using one or more cameras. In some embodiments, electrical parameters in one or more components of a fluid system, e.g., capacitance and/or resistance, may be monitored to detect presence of one or more tissue cores. In some embodiments, an acoustic signal generated by an impact of one or more needles on skin tissue may detected and monitored. An acoustic signal may vary depending on the presence of a core in one or more needles. In some embodiments, information from parameters monitored as described herein may also be used to detect worn or damages needles and/or restricted and/or occluded needle lumens.

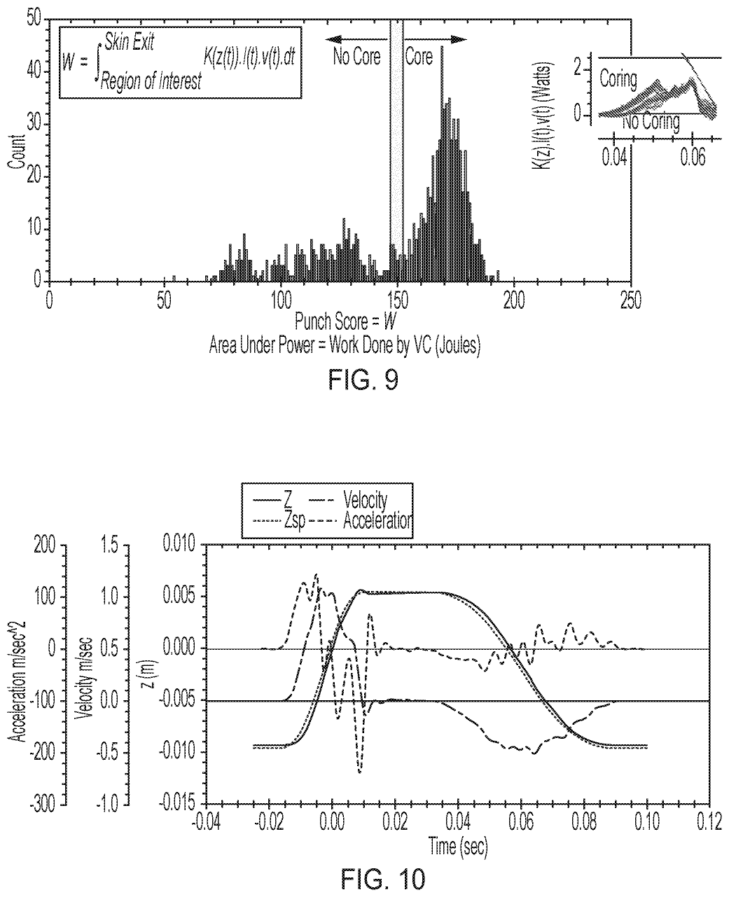

In some embodiments, if a core failed to be extracted, a user may be informed of a coring deficiency, e.g., a digital control unit may receive and process data indicating unsuccessful coring as described above and may generate an output signal to a user interface, e.g., to display a warning to a user. A user may then examine one or more components of a system, e.g., a needle hub, to determine whether there is an obstruction/clog. A user may select a deeper needle depth, e.g., to improve coring efficacy and/or efficiency. Without wishing to be bound by theory, by monitoring the total energy required to withdraw one or more needles it may be possible to determine whether one or more cores were fully extracted. For example, if one or more needles fail to penetrate through full dermal thickness, e.g., into a fat layer, then one or more cores may not be released from the underlying (dermal) tissue. This may result in a decrease in the force (energy) required to withdraw one or more coring needles. FIG. 8 shows an example plot of longitudinal displacement (z-axis displacement) of a voice coil during a coring procedure against time, and electrical power (Voltage (V)×Current (I)×Voice coil constant (K)) consumed by the voice coil against time. Power measured during voice coil actuation shows that the power required to retract a needle with a core may be higher than the power required to retract a needle without core. FIG. 9 shows an example histogram of number of coring strokes (e.g., a stroke being a single z-actuation cycle of a needle hub) against area of under a power curve (e.g., as shown in FIG. 8) as a measure of work done by a voice coil, e.g., of a z-actuator. Work done by a voice coil of a z-actuator during a successful coring cycle may be higher than work done by a voice coil when a needle enters and exits skin tissue without removing a core, e.g., without a core in a needle lumen. Insufficient (partial) coring or failure to core may thus be detected, and a digital control unit may provide a message or warning to a user, e.g., via a display or an audible tone, or both.

In some embodiments, depth of needle penetration may be controlled, e.g., digitally controlled, e.g., using a digital control unit. In some embodiments, depth of needle penetration may be digitally controlled with a backup of one or more mechanical limit stops. In some embodiments, a digital control unit may be used to monitor voice coil data (e.g., position, velocity, and/or acceleration of a voice coil), current draw (e.g., indicating load on a needle), and/or voltage, e.g., to derive depth of penetration from voice coil data. This may allow detection of location of tissue and stop needle progression at a pre-selected depth, e.g., by accelerating or decelerating a voice coil or a moving component thereof.

In some embodiments, movement (displacement) of a z-actuator and/or of a (moving component of a) voice coil may be monitored, e.g., using one or more linear sensors (e.g., one or more encoders, e.g., a z-axis encoder) and/or one or more homing sensors (e.g., one or more optical sensors), e.g., to detect when a z-actuator is completely retracted (e.g., when a moveable component of a voice coil actuator is in the most proximal position away from a skin surface, e.g., linear displacement in direction of a skin surface is zero). In some embodiments, an amount of kinetic energy in a moving voice coil is matched to an amount of energy required to penetrate skin and/or reach a desired depth. In some embodiments, an open-loop control system may be used to control depth based on kinetic energy. In some embodiments, a reference accelerometer may be mounted on or connected to a different component of an apparatus or a hand piece, e.g., on the hand piece shell, to provide data to the digital control unit, e.g., to account for device movement.

Impact on hard tissue may occur as a result of over-penetration, e.g., penetration beyond a dermal layer and/or subcutaneous fat layer. In some embodiments, a controller, e.g., a digital control unit, may be used to monitor discrepancies between commanded z-axis position and actual z-axis position of a z-actuator and/or a voice coil, and/or may be used to monitor deceleration of the z-actuator and/or a voice coil. In some instances, discrepancies between commanded z-axis position and actual z-axis position may occur, e.g., due to a needle impacting an impenetrable structure prior to reaching commanded depth. In some embodiments, if such a discrepancy may be detected and/or if deceleration exceeds a certain threshold, a warning notice may be conveyed to a user (e.g., by a digital processing unit via a display), e.g., if deceleration and/or the amplitude of a an acceleration/deceleration curve exceeds a certain threshold of about, e.g, 10 m/s², 20 m/s², 30 m/s², 40 m/s², 50 m/s², 60 m/s², 70 m/s², 80 m/s², 90 m/s², 100 m/s², 200 m/s², 300 m/s², 400 m/s², 500 m/s², or 1000 m/s².

FIG. 10 shows an example plot of voice coil velocity, position, and acceleration against time during an example normal coring procedure. The commanded z-axis position matches the actual z-axis position. Also, deceleration is less than about 250 m/s².

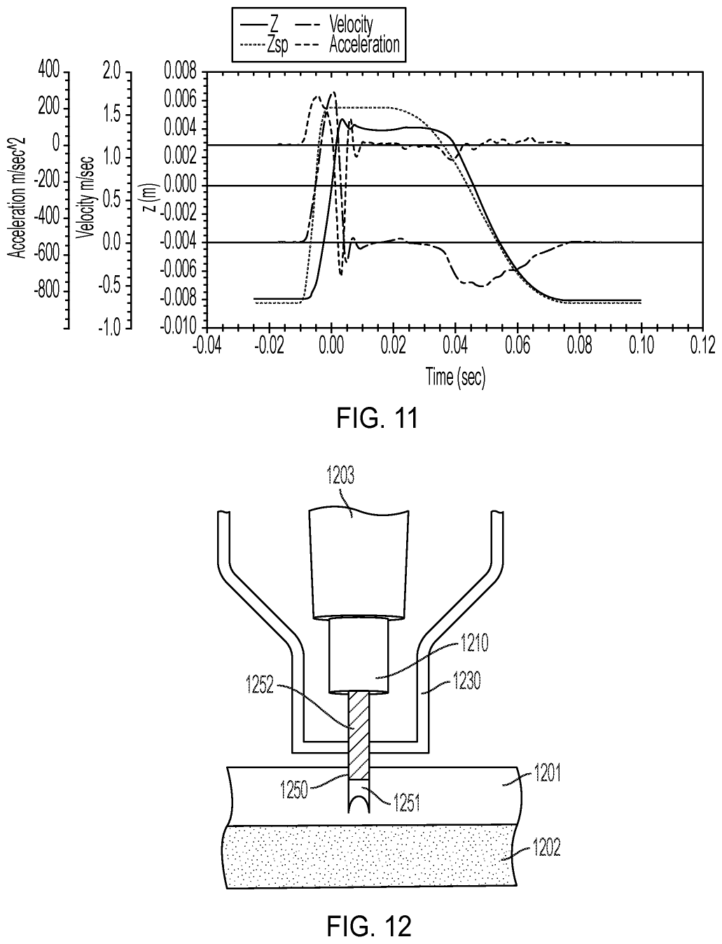

FIG. 11 shows an example plot of voice coil velocity, position, and acceleration against time before, during, and after a coring procedure with excessive over-penetration and contact with hard tissue resulting in a deceleration at impact of about 600 m/s². In this example, a needle tip was severely damaged. In some embodiments, a digital control unit may be used to monitor deceleration and may be used to provide a fault notice to a used, e.g., via a display. Measured decelerations greater than a certain threshold (e.g., 10 m/s², 20 m/s², 30 m/s², 40 m/s², 50 m/s², 60 m/s², 70 m/s², 80 m/s², 90 m/s², 100 m/s², 200 m/s², 300 m/s², 400 m/s², 500 m/s², or 1000 m/s²) may result in termination of a needle lifetime. In some embodiments, a needle hub may be identified, e.g., upon mounting on a needle hub mount, e.g., through a signal received by a digital control unit from a Radio Frequency Identification (RFID) chip located on or in a needle hub. In some embodiments, a digital control unit may be used to block use of an apparatus, e.g., actuation of a z-actuator, until a needle hub including one or more damaged needles is replaced, e.g., as indicated by the removal of the RFID chip associated with a (damaged) needle hub and mounting of a needle hub with a different RFID chip.

Depth Control

In some embodiments, an apparatus as described herein, for example apparatus 100, 200, or 400, may include one or more features or settings that may be used to control or change the depth of penetration of a hollow needle into the skin, e.g., by controlling one or more parameters of a z-actuator (e.g., z-actuator 103, 203, or 403). In some embodiments, an adjustment implement, e.g., a scroll wheel, e.g., on a user interface of a base unit, may be used to adjust an allowed depth of penetration by a hollow needle into skin. In some embodiments, an allowed depth adjustment may be carried out by physically adjusting (e.g., retracting) a hollow needle, e.g., by adjusting position of a hollow needle relative to a distal end of an apparatus, e.g., when a z-actuator is fully retracted (at a most proximal position of an actuation cycle), e.g., by adjusting a position of a stationary base component of a z-actuator. In some embodiments, an adjustment implement, e.g., a scroll wheel on a user interface of a base unit, may be used to provide an electrical signal to a z-actuator to control depth of penetration. In some embodiments, a digital control unit including a user interface of a base unit may control depth and/or timing of penetration into and retraction out of skin by a hollow needle. For example, an operator may program a computer component of a base unit to require a certain displacement of a needled hub and/or a hollow needle into skin based upon an area being treated. A z-actuator as described herein may be programmed or otherwise set to displace a hollow needle up to about, e.g., 10 mm into thick skin (e.g., on a patient's back or into scar tissue), or about, e.g., 1 mm into thin skin (e.g., on a patient's cheeks), for instance. A z-actuator as described herein may be programmed or otherwise set to displace a hollow needle to extend (i) into a dermal layer, (ii) through the entire dermal layer to the junction of the dermal layer and the subcutaneous fat layer, or (iii) into the subcutaneous fat layer.

In some embodiments, a feedback and/or depth control system that may be used with the technologies described herein (e.g., apparatuses 100, 200, or 400) may include an electrically insulated needle. In some embodiments, a coring needle may be electrically insulated (e.g., an external surface of the needle maybe electrically insulated, e.g., by an insulating coating) except for a distal tip, e.g., the needle may not be insulated (exposed) along a length of about 0.2 mm, 0.5 mm, 0.7 mm, 1 mm, 2 mm, 3 mm, 4 mm, or 5 mm from a distal end of a needle for contacting skin. An example insulated needle 1250 mounted on an example needle hub 1210 connected to example z-actuator 1203 and passing through an example spacer 1230 is shown in FIG. 12. An electrical signal, e.g., a radiofrequency (RF) signal, may be applied to a needle (e.g., needle 1250) having a tip (e.g., tip 1251) and an insulated lumen or body (e.g., body 1252). Electrical feedback (e.g., change voltage, current, and/or impedance) from the needle tip may be monitored as the needle penetrates skin of a subject. Without wishing to be bound by theory, once a needle (e.g., a needle tip, e.g., tip 1251) passes through a dermal layer 1201 and begins entry into a fat layer 1202, a measurable change in impedance detected may occur at the tip, e.g., due to a difference in electrical properties between tissue types. In some embodiments, this change in impedance may be used as z-axis position/depth indicator and may be used for feedback, e.g., transmitted to a digital control unit, which in turn may use impedance information to generate or trigger a signal to a z-actuator, e.g., to control or adjust depth of penetration. For example, once a desired full dermal thickness depth has been reached by a needle tip, impedance feedback to a digital control unit may cause the unit to signal a z-actuator to stop and reverse (withdraw) from a patient's dermis.

In some embodiments, a system as described herein may include a control system, e.g., a digital control unit, that may be used to monitor voice coil data, e.g., position, velocity, acceleration, current draw, and/or voltage. A digital control unit may be used to control (e.g., accelerate or decelerate) voice coil of a z-actuator based on pre-programmed commands and signals, and/or based on signals from a depth control system, e.g., a depth control system including an electrically insulated needle. In some embodiments, voice coil actuator and/or z-actuator movement may be monitored using a linear sensor (e.g., an encoder) and/or a homing sensor (e.g., an optical sensor) that may detect when a moveable component of a voice coil actuator or z-actuator is completely retracted away from a skin surface, e.g., is at a most distal position in an actuation cycle. In some embodiments, a vision system, e.g., using a camera, may be used to monitor needle travel. A reference accelerometer in or on an apparatus or hand piece, e.g., in or on a hand piece shell, may provide input data to a digital control unit, e.g., to account for device movement. In some embodiments, a digital control unit may be programmed to match an amount of kinetic energy in a voice coil to an energy required for a needle hub to reach a certain distance, e.g., for a needle to reach a certain depth.

In some embodiments, a needle may be advanced further until an exposed RF tip, e.g., needle tip 1251, is disposed entirely within the fat layer, or until the RF tip has reached a predetermined depth in the fat layer (e.g., 1 mm, 2 mm, or 3 mm depth of fat layer). This may be useful for applications where it is desired to remove fat as well as skin cores. In some embodiments, a hypodermic needle, e.g., a needle of less than 1 mm in internal diameter, which is not intended to core skin, may be advanced into and through the dermis of a subject and into a fat layer below, which may result in a hole through the subject's dermis, but not a core. In some embodiments, fat or other tissue beneath the dermis may be withdrawn via a needle lumen, e.g., using a liposuction procedure. In some embodiments, a signal from an RF tip that a fat layer was reached may be used as an input signal to a digital control unit that may be used to activate a tissue suction mechanism.

In some embodiments, an apparatus may include or may be connected to one or more of depth control systems that may include one or more skin surface or layer detection technologies. Skin surface or skin layer detection technologies may include systems and/or methods to monitor capacitance in a needle and detect changes therein to infer needle position/depth relative to a skin layer. Skin surface or skin layer detection technologies may include acoustic technologies, e.g., a microphone that may be used to ‘hear’ impact of one or more needles on a skin surface. Skin surface or skin layer detection technologies may include visual systems (e.g., one or more cameras) to detect and/or monitor skin surface location and/or needle/voice coil travel.

In some embodiments, a depth control system may include one or more technologies for detection of a dermal/fat interface, e.g., to control (e.g., stop) needle progression. Capacitance changes from air to dermis to fat, and may be detected and/or monitored using technologies analogous to technologies for impedance detection and/or monitoring, e.g., using one or more insulated or partially insulated needles, e.g., using polyvinylidene fluoride (PVDF) as an insulating material.

In some embodiments, a depth control system may include one or more technologies that employ ultrasound, optical coherence tomography (OCT), or other acoustic or vision based technology to assess depth of penetration by one or more needles, such as penetration of the fat/dermal interface. In some embodiments, dermal layer thickness may be determined by evaluating a previously removed core by vision, acoustic, or electrical systems or methods.

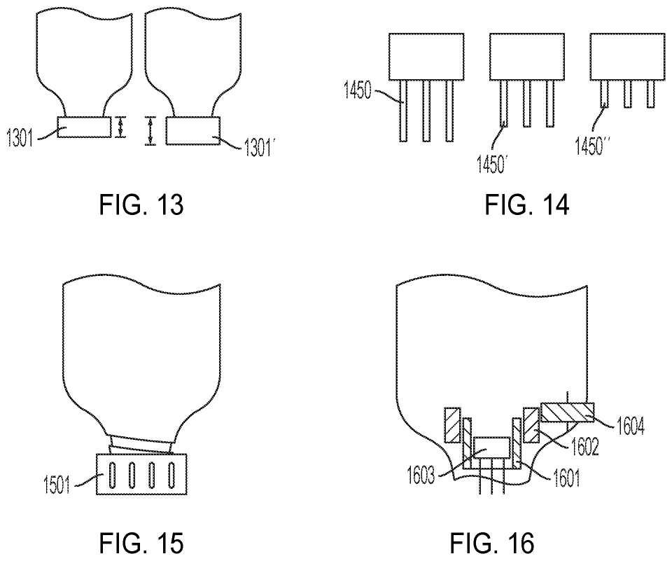

In some embodiments, mechanical depth control technologies may be used with the technologies described herein. Mechanical depth control technologies may include one or more depth control spacers, e.g., depth control spacer elements attached to a spacer frame as described herein, or other movement limitation implements that may limit z-actuation of a needle hub. In some embodiments, mechanical depth control technologies may be used alone or in combination with electrical technologies, e.g., as described above. FIG. 13 shows two embodiments of an example apparatus as described herein, wherein each embodiment has a depth control spacer element 1301 or 1301′ with a different length at a distal end of an apparatus, e.g., at or on a (vacuum) spacer frame as described herein. At constant needle length and actuation distance, a longer depth control spacer element may cause a shallower penetration depth as a greater needle actuation distance is covered by a depth control spacer element. FIG. 14 shows three different example needle hub configurations with needles, e.g., example needles 1450, 1450′, and 1450″, of different length to control depth of penetration. FIG. 15 shows an example embodiment of an example apparatus as described herein, where a depth control spacer element 1501 is threaded onto a tubular region at a distal end of an example apparatus. Depth of penetration of a needle may be controlled by adjusting position of a depth control spacer element, e.g., by rotating a depth control spacer element on a threaded end region of an apparatus. FIG. 16 shows an example embodiment of an example apparatus as described herein including a mechanism, e.g., an internal threaded mechanism, to raise or lower (relative to a skin surface during operation) an actuation unit that may be or include a z-actuator, e.g., z-actuator 1603, and/or a moveable component of a voice coil actuator. In some embodiments, an internal threaded mechanism is or includes a rack and pinion or rack and worm arrangement, e.g., rack 1601 and worm 1602. In some embodiments, an internal threaded mechanism may be manually actuated (e.g., through a wheel, e.g., on a hand piece shell, e.g., wheel 1604), or may be actuated through a motor.

Recoil Compensator

As a z-axis voice coil (or moving component thereof) accelerates and decelerates, a counter force may be imparted to an apparatus, including, e.g., a hand piece (e.g., hand piece 120, 220, or 420) and/or hand piece shell (e.g., hand piece shell 121, 221, or 421) encasing an actuation unit comprising a z-actuator. A hand piece may be held by a user operator and may be configured for optimal ergonomics. In some embodiments, a hand piece and its components (e.g., a hand piece shell) are made as light as possible, e.g. for user comfort. Without wishing to be bound by theory, a lower mass of the apparatus may worsen the recoil effect felt in the hand piece due to reduced inertia of the apparatus. In some embodiments, multiple needles, e.g., a needle array, may be used. The greater the number of needles on a given needle hub, the correspondingly greater acceleration may be required to drive the needles into or through the patient's dermis, e.g., to obtain a full thickness core, which may worsen user-felt recoil. An apparatus as described herein, e.g., an apparatus equipped with an ultra-light hand piece and/or a needle hub with multiple needles, may benefit from a recoil compensating mechanism, which may improve user experience and/or positional stability of a hand piece, e.g., by moving a mass counter to a z-axis stroke and cancelling or diminishing user felt recoil.

In some embodiments, a z-actuator may include multiple voice coil actuators. In some embodiments, a z-actuator comprises dual countering voice coils or voice coil actuators arranged along their axis of movement. Dual countering voice coils may be used such that one voice coil (or moving component thereof) cancels or reduces an effect of a change in momentum of the other voice coil (or moving component thereof) during operation. In some embodiments, a z-actuator may include or may be connected to a recoil compensator, e.g., a counter balance mass to reduce the effect of a change in momentum of a voice coil (or moving component thereof) during operation.

In some embodiments, an apparatus may include a hand piece accelerometer (e.g., mounted on or connected to the hand piece shell), which may provide feedback to a system (e.g., a digital control unit) including, e.g., a z-axis counter mass controller. A z-axis counter mass controller may be used to minimize accelerations detected by the hand piece accelerometer. In some embodiments, a z-axis counter mass controller may include a counter mass weight, e.g., a piece of metal, which may be moveably mounted in or on the apparatus, e.g., in or on a hand piece, e.g., a hand piece shell, in a direction substantially parallel and/or opposite to the direction of motion of a voice coil (or moving component thereof) of a z-actuator and/or a needle hub displaced by the z-actuator. A z-axis counter mass controller may include a motor for moving the counter mass weight and may include an electronic control system. In some embodiments, an electronic control system (e.g., a digital control unit) may be used to monitor movement of the apparatus, e.g., the hand piece, e.g., based on data obtained from the accelerometer, and to move the counter mass weight in a direction opposite to the direction of movement of the apparatus, e.g., the hand piece. In some embodiments, an electronic control system may be used to monitor movement of a needle hub and/or voice coil of a z-actuator and to move the counter mass weight in a direction opposite to the direction of movement of the needle hub and/or voice coil. In some embodiments, the z-axis counter mass weight may be of equal weight and may be moved with equal but opposite acceleration and/or velocity as the voice coil (or moving component thereof) of a z-actuator, which may cancel recoil caused by movement of the z-axis voice coil, without acceleration feedback from an accelerometer. In some embodiments, a counter mass weight may travel the substantially same distance at the substantially same velocity as a voice coil (or moving component thereof) of a z-actuator or a needle hub displaced by a z-actuator. In some embodiments, a counter mass weight may act to reduce rather than cancel recoil felt by an operator of the apparatus. In an example embodiment, a z-axis counter mass weight may travel in a direction opposite to the direction of movement of a voice coil (or moving component thereof) of a z-actuator or a needle hub displaced by a z-actuator, but only by a fraction of the distance of movement of a (moving component of a) voice coil of a z-actuator or a needle hub displaced by a z-actuator. This may reduce the worst “edges” of felt recoil, which may occur at an end of travel distance of a voice coil (or moving component thereof) of a z-actuator or a needle hub displaced by a z-actuator, which may be the period of maximum acceleration or deceleration. In some embodiments, a recoil compensating counter mass weight may be driven by a voice coil actuator substantially similar to the z-actuator, wherein the recoil compensating voice coil actuator is arranged to move the reciprocate counter mass weight in a direction opposite to the direction of travel of a voice coil (or moving component thereof) of a z-actuator or a needle hub displaced by a z-actuator.

In some embodiments, a z-actuator may be configured to maintain an apparatus or a component thereof at a low temperature (e.g., less than about 43° C., such as less than about 43, 42, 41, 40, 39, 38, 37, 36, or 35° C.) to avoid subject and/or user discomfort and/or to avoid damage to the skin tissue (e.g., collagen in the skin tissue is sensitive to high temperatures, e.g., temperatures above 40° C.). Actuator types having characteristics for maintaining a low temperature include voice coil actuators, pneumatic actuators, electromagnetic actuators, motors with cams, motors with lead screws (e.g., stepper motors), and piezoelectric actuators. In some embodiments, a low temperature z-actuator is a voice coil actuator.

XY-Actuator

In some embodiments, an apparatus as described herein may include an “x” and/or “y” actuator (e.g., an x/y actuator) for translating a needle hub and/or one or more hollow needles across skin, e.g., x-actuator 101, 201, or 401 and/or y-actuator 102, 202, or 402. An x/y-actuator may be used to establish skin treatment coverage. In some embodiments, a x/y-actuator may have a relatively small displacement range (e.g., maximum distance between a first x/y position and a second x/y position), e.g., less than about 10 mm (e.g., 10, 9, 8, 7, 6, 5, 4, 3, 2, or 1 mm). In some embodiments, an x/y-actuator may have a relatively large displacement range (e.g., up to about 30 mm). An x/y-actuator may operate with high positional accuracy (e.g., distance between a selected position and actual position, e.g., of a hollow needle). For example, an x/y-actuator may position a hollow needle to penetrate skin within a 30 μm radius (e.g., within 30, 25, 20, 15, 10, or 5 μm) of a selected position. An x/y-actuator may operate with high position accuracy that may allow continuous treatment across a treatment area. High position accuracy may provide the ability to re-enter a hole previously created and/or repeat coring at a position previously targeted, e.g., if coring was not achieved completely. In some embodiments, a needle may re-enter a hole previously created or previously targeted by the same needle, e.g., without translation in the x or y direction between the two entries. In some embodiments, a needle may enter a hole previously created or previously targeted by a different needle. In some embodiments, to deliver a drug or other substance to the hole, a needle may re-enter a hole previously created.

A treatment area may be a skin area that contains multiple treatment sites, e.g., a 3 cm by 3 cm treatment area containing nine 1 cm² treatment sites. An x/y-actuator may facilitate movement of a needle hub and/or one or more hollow needles of an apparatus from one treatment site to an adjacent treatment site within a treatment area. An x/y-actuator may facilitate movement of a needle hub and/or one or more hollow needles of an apparatus within each treatment site. An x/y-actuator may operate with high position accuracy that may avoid gaps between adjacent treatment sites in a treatment area and/or avoid overlaps between adjacent treatment sites in a treatment area. In some embodiments, an x/y actuator may enable creation of different hole patterns. In some embodiments, a hole pattern may be regular or irregular, uniform or non-uniform. Regular patterns include rows and/or arrays of equally spaced holes. Irregular patterns include random patterns. Uniform patterns include rectangular or arrays of equally spaced holes. Non-uniform patterns include arrays with differently spaced holes. In some embodiments, a pattern can be pre-set or pre-programmed, e.g., to match tissue conditions and/or desired treatment effect. In some embodiments, a pattern may be altered or modified during operation of the device. Array patterns that may be generated with the technologies described herein are described in detail below

An x/y-actuator may also operate at a relatively high speed to minimize treatment time. In some embodiments, one actuation cycle in the x- and/or y-direction may take from about 50 milliseconds to about 250 milliseconds (e.g., 50, 75, 100, 125, 150, 175, 200, 225, or 250 milliseconds). In some embodiments, one actuation cycle in the x- and/or y-direction may take about 120 milliseconds to about 160 milliseconds (e.g., 120, 125, 130, 135, 140, 145, 150, 155, or 160 milliseconds (e.g., about 140 milliseconds)). In some embodiments, one actuation cycle in the x- and/or y-direction may take about 120 milliseconds to about 160 milliseconds (e.g., 120, 125, 130, 135, 140, 145, 150, 155, or 160 milliseconds (e.g., about 140 milliseconds)) to travel about 0.6 mm to about 1 mm (e.g., 0.6, 0.65, 0.7, 0.75, 0.8, 0.85, 0.9, 0.95, or 1 mm). In some embodiments, one actuation cycle in the x- and/or y-direction may take about 140 milliseconds to travel about 0.833 mm.

In some embodiments, an x/y-actuator may be capable of operating with a force of about 0.5 N to about 20 N (e.g., 0.5 N to 0.75 N, 0.5 N to 1 N, 0.5 N to 1.25 N, 0.5 N to 1.5 N, 0.5 N to 2 N, 0.5 N to 5 N, 0.5 N to 10 N, 0.5 N to 12 N, 0.5 N to 15 N, 0.5 N to 20 N, 0.75 N to 1 N, 0.75 N to 1.25 N, 0.75 N to 1.5 N, 0.75 N to 2 N, 0.75 N to 5 N, 0.75 N to 10N, 0.75 N to 12 N, 0.75 N to 15 N, 0.75 N to 20N, 1N to 1.25N, 1N to 1.5N, 1N to 2N, 1N to 5N, 1N to 10N, 1N to 12N, 1N to 15N, 1N to 20N, 1.25 N to 1.5 N, 1.25 N to 2 N, 1.25 N to 5 N, 1.25 N to 10N, 1.25 N to 12 N, 1.25 N to 15N, 1.25N to 20N, 1.5N to 2N, 1.5N to 5N, 1.5N to 10N, 1.5N to 12N, 1.5N to 15N, 1.5N to 20N, 2 N to 5N, 2N to 10N, 2N to 12N, 2N to 15N, 2N to 20N, 5N to 10N, 5N to 12N, 5N to 15N, 5N to 20N, 10N to 12N, 10N to 15N, 10N to 20N, 12N to 15N, 12N to 20N, or 15N to 20N) per hollow needle can be applied to translate the needle across the skin. In some embodiments, a force of about 5 N to 15 N (e.g., 10 N) per hollow needle may be applied to translate a needle across skin.

An x/y-actuator may be configured to maintain an apparatus or a component thereof at a low temperature (e.g., less than about 43° C., such as less than about 43, 42, 41, 40, 39, 38, 37, 36, or 35° C.) in order to avoid raising the apparatus temperature to a level that could cause subject and/or user discomfort. Actuator types having characteristics for maintaining a low temperature include voice coil actuators, pneumatic actuators, electromagnetic actuators, motors with cams, piezoelectric actuators, and motors with lead screws (e.g., stepper motors). In some embodiments, an x/y-actuator is a stepper motor with a lead screw.

In some embodiments, one or more components of an apparatus as described herein may be selected or designed to secure a needle hub and/or one or more hollow needles and/or prevent or minimize angular movement (e.g., wobbling) of the hollow needle(s). In some embodiments, an x-, y-, and/or z-actuator may operate without causing any significant angular movement (e.g., wobbling) of a needle hub and/or one or more hollow needles. In some embodiments, a z-actuator may insert and/or withdraw one or more hollow needles in a linear fashion without any significant angular movement (e.g., wobbling) of the one or more hollow needles. A hollow needle may be secured to a needle hub so as to minimize or reduce angular movement of needle(s) during insertion to less than 5 degrees, e.g., less than 4, 3, or 2 degrees. An angular movement of a needle during insertion of −1-1.5 degrees may be within nominal tolerances, whereas an angular movement of the needle during insertion of −4-5 degrees or more may need to be avoided, if possible. In some embodiments, components that join one or more hollow needle(s) to other components of the needle assembly, e.g., a needle hub, may be designed with low mechanical tolerances to firmly secure the one or more hollow needles. This may reduce prevalence of or may lower the risk of destabilization and/or reduction in the structural integrity of hollow needle(s) that may result from repeated use. Firmly securing needle(s) may prevent and/or minimize dulling, bending, and curling of needle tip(s) that could reduce the effectiveness of the needle(s). Firmly securing needle(s) may also reduce the risk of over-striking (e.g., striking a hole produced by a needle again).

In some embodiments, actuators, e.g., z-, x-, and y-actuators, may be activated independently or together by one or more buttons, keys, toggles, switches, screws, dials, cursors, spin-wheels, or other components. In some embodiments, each of the z-, x-, and y-actuators can be separately controlled (e.g., using separate activation components, such as a button, or by using separate controls in a user interface). In some embodiments, an apparatus includes a multiplexer, e.g., to select one or more input signals or output signals, e.g., from or to an actuator or sensor, and transmit a signal in a single line.

Rotary Stage

In some embodiments, an apparatus and/or an actuation unit as described herein may be or include a rotary stage, e.g., to rotate a needle hub around an axis perpendicular to a surface of skin to be treated, e.g., around a z-axis. A rotary stage may include one or more motors and/or actuators, e.g., an electrical motor, e.g. a stepper motor. In some embodiments, a rotary stage is or comprises a z-actuator, e.g., as described above, and/or a rotation mechanism.

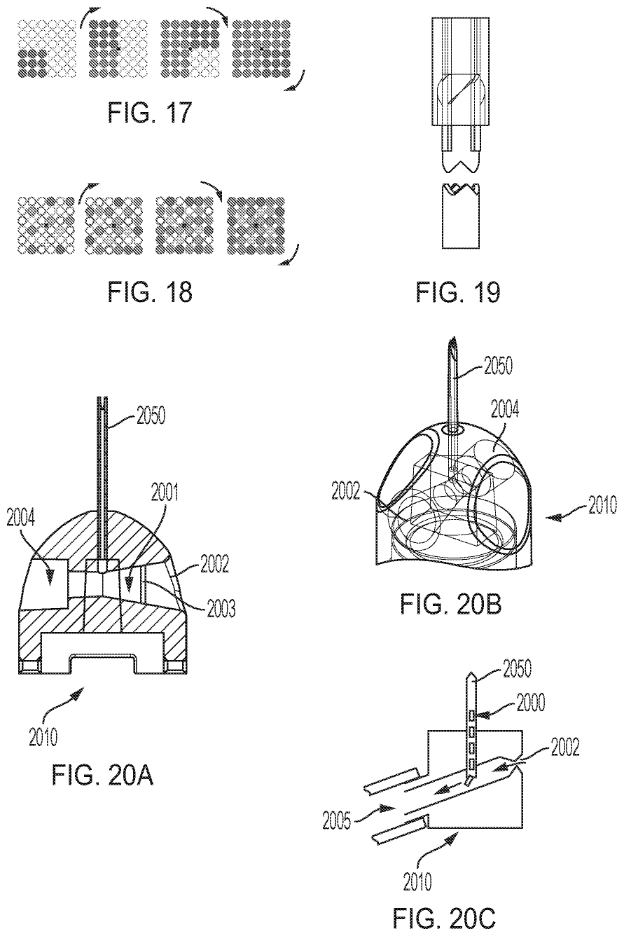

In some embodiments, a movement of or by a z-actuator may cause a needle hub and/or one or more needles, e.g., a needle array, to rotate, e.g., by about 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, or 90 degrees, and/or rotate by about 100 degrees, 110 degrees, 120 degrees, 130 degrees, 140 degrees, 150 degrees, 160 degrees, 170 degrees, 180 degrees, 200 degrees, 210 degrees, 220 degrees, 230 degrees, 240 degrees, 250 degrees, 260 degrees, 270 degrees, 280 degrees, 300 degrees, 310 degrees, 320 degrees, 330 degrees, 340 degrees, 350 degrees, or 360 degrees. In an example embodiment, each movement of or by a z-actuator may cause a needle hub and/or one or more needles, e.g., a needle array, to rotate, e.g., around a z-axis of a z-actuator. In an example embodiment, a 3×3 needle array may be rotated by 90 degrees during each actuation of a z-actuator. In this example, a pattern may have 4 quadrants, and four z-actuations (or strokes) may comprise a complete pattern, as shown in FIG. 17 and FIG. 18. In an example embodiment, an apparatus may be used for or may be configured for sequential patterning, e.g., all needles are acting on the same quadrant or equivalent, e.g., a sector of any size or shape (e.g., triangular, rectangular, or polygonal) (see FIG. 17). In an example embodiment, an apparatus may be used or configured for concurrent patterning, e.g., needles may act on the two or more different quadrants or equivalent (e.g., sectors of any size or shape). FIG. 18 illustrates example concurrent patterning: a partially filled circle indicates a hole created during a current z-actuation, and a completely filled circle indicates a hole created during a previous z-actuation. An apparatus as described herein may be configured for any number of strokes to complete a pattern of holes.

In some embodiments, a rotation mechanism may be used that includes a single planar translation mechanism, e.g., translation along a radius of a circle. Instead of encoding a position of a needle hub and/or z-actuator in Cartesian coordinate system (x, y), a position of a needle hub and/or a z-actuator may be encoded in polar coordinates (e.g., radius r, angle theta). In some embodiments, use of a rotation mechanism with two degrees of freedom may eliminate the need for x/y-translation and thus a need for an x-actuator and/or a y-actuator. This may lead to reduced weight of an apparatus, reduced size of a hand piece, and/or reduced cost. Reduction of hand piece size, e.g., hand piece shell diameter, may be an advantage to users with respect to ease of use of an apparatus as described herein. In some embodiments, an apparatus as described herein may be an ultra-fine precision, light-weight, and low cost coring apparatus that may include a rotary stage or a retractable “pen-click”-type rotary mechanism, e.g., as shown in FIG. 19, and a recoil compensator (e.g., a counter mass moving opposite to the z-actuator to reduce or eliminate hand piece movement due to z-axis acceleration/deceleration).

Needle Hub

The technologies described herein may include a system and/or apparatus that includes a needle hub. In some embodiments, a needle hub may be or include a needle hub assembly comprising one or more needle joints, e.g., to receive and/or hold one or more (hollow) needles. In some embodiments, a needle hub may include a first lumen having a wall, a first end and a second end. A first lumen may include, or may be in fluid communication with, a lumen of a hollow needle, e.g., wherein the first end of the first lumen is at a distal end of the hollow needle for contacting skin.

In some embodiments, a needle hub may be or include a needle hub assembly including two or more lumens, e.g., in fluid communication with each other. In some embodiments, a needle hub may include a second lumen having a wall, a first end, and a second end. In some embodiments, a second lumen may be in fluid communication with a first lumen, e.g., wherein the first lumen may include, or may be in fluid communication with, a lumen of a hollow needle. In some embodiments, a first lumen may be connected to a second lumen forming a junction such that the second end of the first lumen forms an opening in the wall of the second lumen. This may facilitate clearing of material, e.g., skin cores, from a first lumen (e.g., from an example hollow needle), as described further below. An example needle hub 2010 with two lumens is shown in FIGS. 20A and 20B.

First and second lumens and/or junctions between a first and second lumen may have any shape and/or configuration. In some embodiments, each of the first lumen and the second lumen may be substantially straight, and the first lumen may be substantially perpendicular to the second lumen forming a T-junction. In some embodiments, the first and second lumen may be connected at an angle, e.g., at about 0 degrees, 10 degrees, 20 degrees, 30 degrees, 40 degrees, 50 degrees, 60 degrees, 70 degrees, 80 degrees, or 90 degrees. In some embodiments, one or both of a first lumen and a second lumen may be curved and/or include a substantially straight and/or a curved section. In some embodiments, a lumen, e.g., one or both of a first lumen and a second lumen, may have a constant diameter along a length of a lumen or may have a diameter varying along a length of a lumen. A lumen may have any cross-sectional shape, e.g., circular, square, oval, rectangular, angular, or any combination thereof.

In some embodiments, a first lumen may include a lumen of a hollow needle (e.g., needle 2050), e.g., wherein the first end of the first lumen is at a distal end of the hollow needle for contacting skin. In some embodiments, the first end of the second lumen may be or include a fluid intake nozzle, e.g., an air intake nozzle 2001. In some embodiments, a fluid intake nozzle may be or include a convergent nozzle, a divergent nozzle, a convergent-divergent nozzle, a cylindrical nozzle, and/or a frusto-conical nozzle. In some embodiments, a second lumen or fluid intake nozzle may include a filter, e.g., filter 2003, to remove impurities (e.g., dust) from fluid traversing the nozzle. In some embodiments, a first end of the second lumen (e.g., a nozzle) maybe exposed to ambient atmosphere, e.g., at inlet 2002. In some embodiments, a first end of the second lumen (e.g., a nozzle 2001) may be connected to a fluid conduit, e.g., at outlet 2004 at the second end of the second lumen. In some embodiments, a first end (e.g., a nozzle 2001) of the second lumen may not be connected to a fluid conduit.

Core Clearing

A needle hub as described herein may be used for or to facilitate removal of tissue from a hollow needle. In some embodiments, a needle hub may be connected to or may be part of a fluid system, e.g., a fluid-based core clearing system, that may be used to facilitate removal of tissue, e.g., one or more skin cores, from one or more needles. As a coring needle is driven into or through the dermis of a subject (and/or into a fat layer below) skin tissue, e.g. one or more full thickness skin cores, is driven up into a needle lumen. During repeated operation with the same needle, multiple skin cores may stack up inside a lumen of a hollow needle and/or a first lumen of a needle hub, and may compress together. Repeated operation may lead to one or more skin cores filling up a lumen of a hollow needle and/or a first lumen of a needle hub. Repeated operation of the same needle may lead to one or more skin cores being pushed out of an opening in the lumen of a hollow needle and/or out of a first lumen of a needle hub (e.g., out of a second end of the first lumen). In some embodiments, a first lumen may be connected to a second lumen as described above, e.g., where a first lumen is connected to a second lumen forming a junction such that the second end of the first lumen forms an opening in the wall of the second lumen. In some embodiments, a second end of a second lumen may be connected to a fluid conduit such that when low pressure or (partial) vacuum is applied to the conduit, low pressure or (partial) vacuum is induced in the first lumen and second lumen, e.g., such that fluid may be drawn into the second lumen through the first end of the second lumen.

Without wishing to be bound by theory, once a core begins to emerge from a first lumen, e.g., a lumen of a hollow needle, and enter a second lumen, the core may be exposed to cross fluid flow in the second lumen (e.g., an airstream, e.g., a high velocity airstream, e.g., a (near) supersonic airstream) and associated drag force. Any fluid may be used, for example any gas (e.g., air, carbon dioxide, or nitrogen gas) or any liquid (e.g., water, saline, an aqueous solution, or oil), or any combination thereof. In some embodiments, fluid flow (e.g., airstream) in a second lumen exerts a lateral (drag) force on a side of a first core emerging from a first lumen, which may pull the core from the first lumen (e.g., as the core is flexible and may bend, thus translating the force acting on the side of the core into a tensional force). In some embodiments, during repeated operation of the same needle, multiple cores may enter a lumen of a hollow needle and/or a first lumen. One or more cores stacking behind (e.g., distally along a first lumen, e.g., a hollow needle lumen) an emerging core (e.g., a first core) in a first lumen may push the emerging core into a second lumen. In some embodiments, a core (e.g., an emerging core) may be exposed to both a lateral force from a fluid stream and force exerted from one or more cores stacking behind the emerging core. In some implementations, a suction force may act on a first lumen (e.g., a lumen of a hollow needle), which may cause one or more cores to be sucked from the first lumen, e.g., into a second lumen. FIG. 20C is a diagram illustrating an example core clearing procedure, e.g., in a needle hub 2010, wherein air is drawn into a second lumen through an air intake 2002 by means of a vacuum source downstream of the second lumen in fluid connection with needle hub 2010 through a vacuum line 2005. One or more skin cores 2000 may be drawn from a first lumen into the second lumen.

In some embodiments, a lumen, e.g., a second lumen, may be configured as a Venturi-like nozzle. Fluid, e.g., air, may be drawn through a nozzle, e.g., a fluid intake nozzle at a first end of the second lumen. In some embodiments, a fluid intake nozzle may include a filter, nozzle inlet, and/or a cross sectional constriction followed by a larger cross section tubing, e.g., a second lumen may be configured as a convergent-divergent duct. In some embodiments, constant airflow may be drawn through the fluid intake nozzle. Fluid flow, e.g., air flow, may accelerate through a convergent part of the lumen, reaching a maximum air velocity at a constriction of a convergent-divergent duct, e.g., the smallest cross sectional area of the lumen. A first lumen may be connected to the second lumen forming a junction such that the second end of the first lumen (e.g., a proximal end of a hollow needle) forms an opening in the wall of the second lumen at or near the constriction. Air velocity across the second end of the first lumen (e.g., a proximal end of a hollow needle) may be sufficiently high to create a low pressure at (e.g., in and/or around) the second end of the first lumen. Low pressure (e.g., pressure below atmospheric pressure) at the second end of the first lumen may create suction in the first lumen, which may cause one or more cores in the first lumen to be drawn towards and/or out of the second end of the first lumen. This process may occur alone or in combination with a force exerted by one or more (stacked) cores drawn into the first lumen, e.g., through a first end of the first lumen, e.g., caused by movement of the first lumen into skin tissue, causing formation of new cores inside the first lumen.

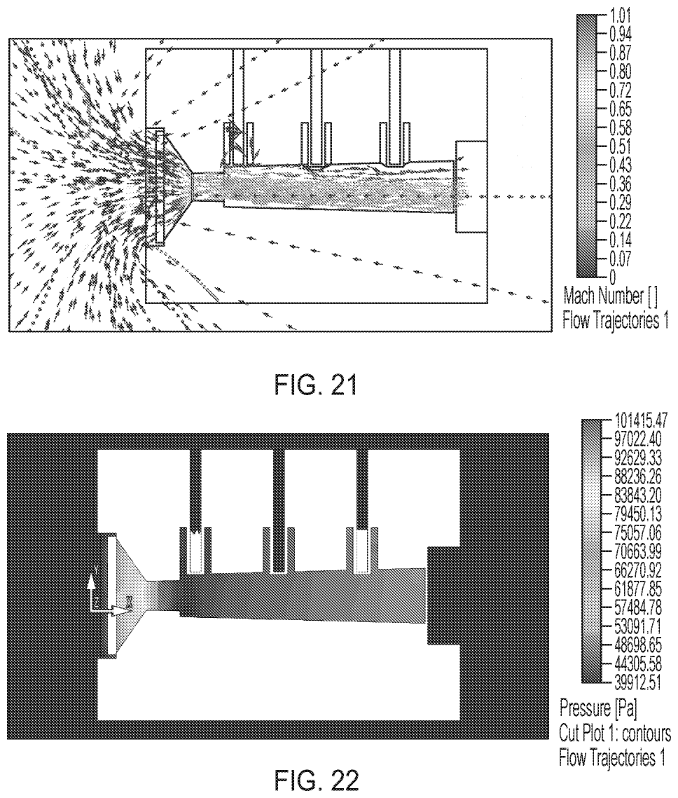

FIG. 21 and FIG. 22 show results of a computational fluid dynamics (CFD) simulation in an example channel (e.g., a second lumen) comprising a “steep” conical/frusto-conical inlet (convergent) and having a longer, “shallow” frusto-conical profile downstream from the inlet (divergent). The example model includes three additional channels (e.g., first lumens) that represent lumens including or connected to lumens of example needles. In this example simulation, coring needle lumens (e.g., first lumens) are blocked off to show flow effects as if cores were stacked up and blocking the needle lumens. Stacked cores may adhere to each other, which may require a very high velocity airflow (e.g., (near) supersonic flow in case of gas), to “break off” each core from the stack of cores. Fluid flow rates and/or velocities may depend on a size of channels. Flow rates of a fluid (e.g., a gas (e.g., air)) may be adjusted such that the maximum Mach number in a channel is about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0. In some embodiments, the maximum Mach number in a channel is about 0.72. In some embodiments, flow in a channel may be supersonic (e.g., the maximum Mach number in a channel is about 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, or 2.0). FIG. 21 shows a cutaway section of the channels where arrows indicate direction of flow. Gray scale of arrows indicate flow velocity ranging from near zero outside the example channels to (near) supersonic flow (Mach 1) at or near the narrowest point of the example second channel FIG. 22 shows a cutaway section of the channels where gray scale indicate fluid pressure. Reduced pressure can be observed in the divergent portion of the example channel, which may indicate a suction force on the first lumens.

As described further herein, an example fluid system including a first and second lumen as described above, may include or be connected to auxiliary technologies, e.g., one or more valves, pumps, filters, tissue traps, tubing, and tubing connectors. Fluid flow (e.g., air flow) through a lumen, e.g., a second lumen, may be controlled to be continuous or pulsed. In some embodiments, fluid flow is pulsed on/off. Pulsed flow may cause change in direction of force acting on a skin core, which may aid dislodgement of a skin core and/or transport in the fluid stream. In some embodiments, a fluid system (e.g., a suction system) may be directly connected to a lumen of one or more hollow needles, e.g., connected to one or more first lumens without a second lumen and without a cross-flow system as described above.

Other configurations and technologies may be used for fluid-based core clearing. In some embodiments, a stream of liquid may be employed instead of a stream of gas (e.g., an airstream) to remove one or more cores. In some embodiments, a closed-loop hydraulic system may be used to draw liquid through a lumen (e.g., a second lumen) to remove one or more cores.

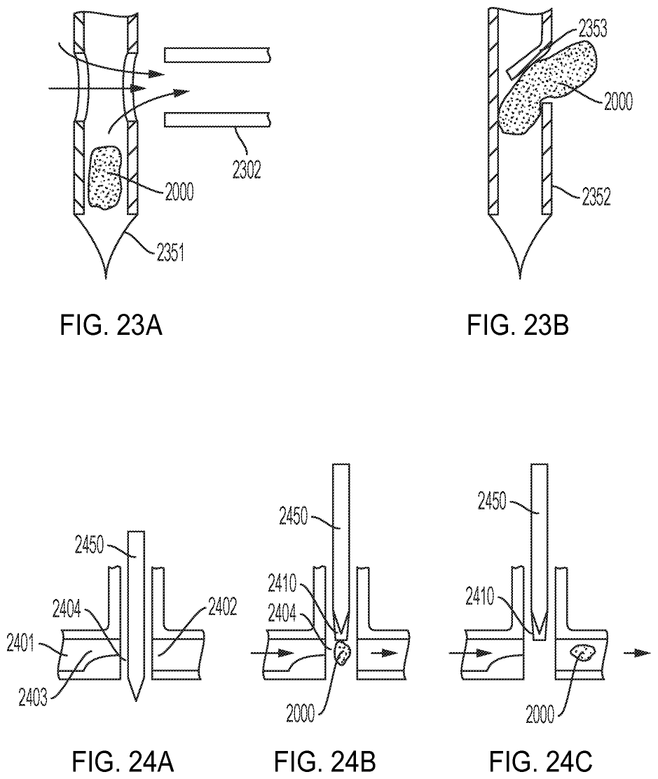

In some embodiments, a needle as used with the technologies described herein and/or a first lumen may have one or more lateral openings. In some implementations, an example needle 2351 and/or a first lumen may have two or more lateral openings, e.g., two openings opposite each other, e.g., as shown in FIG. 23A. In some embodiments, a fluid device, e.g., a suction device or manifold 2302, may be placed on or near a first lateral opening. A negative pressure or suction force may be applied in the fluid device, e.g., suction device. Negative pressure or suction force may be induced in the second opening and/or in the needle lumen and/or in the first lumen. One or more skin cores 2000 inside a needle lumen and/or a first lumen may be drawn up and/or out, e.g., through the first opening, by suction force. In some embodiments, a lateral opening may include or may be connected to a protrusion 2353 into a lumen of an example needle 2352 and/or a first lumen, e.g., to guide one or more tissue cores 2000 out of the needle lumen and/or first lumen, e.g., as shown in FIG. 23B. In some embodiments, cores 2000 may be cleared from a needle by simple stacking: older cores may be pushed out by subsequent cores generated by repeated insertion of the needle, e.g., pushed out of a lateral opening, e.g., as shown in FIG. 23B. In some embodiments, no suction may be used to remove a core 2000 from a lateral opening.

In some embodiments, one or more cores 2000 may be removed from a lumen, e.g., a first lumen, by an internal removal tool, e.g., a pushrod. In some embodiments, an internal tissue removal tool may be a piston or a pin that fits inside the lumen of a hollow needle (e.g., without creating a (partial) vacuum inside the lumen (e.g., the gap between the tissue removal tool and the wall of the lumen of the hollow needle may be large enough to allow the passage of air)). In some embodiments, an internal removal tool may be a piston. A removal tool (e.g., a piston or pushrod) may not disrupt a structural integrity of a cored tissue portion. In some embodiments, an internal removal tool (e.g., a piston or pushrod) may push one or more cored tissue portions out of a lumen of a first lumen (e.g., a hollow needle) as a substantially intact, cored tissue portion, instead of as pieces of the cored tissue portion, which may be difficult to remove completely. Maintaining structural integrity of a cored tissue portion as a substantially intact tissue portion during a removal process may facilitate efficient and complete tissue removal from a hollow needle.

In some embodiments, a tissue removal tool may be combined with a fluid system, e.g., a fluid based core removal system. In some embodiments, an example needle 2450 as described herein may retract from skin into a needle channel such that a needle tip may be positioned in or adjacent to a suction manifold, e.g., manifold 2403, comprising a manifold lumen, e.g., as shown in FIG. 24A. In some embodiments, a manifold, e.g., manifold 2403, may be configured with two intakes. A first intake, e.g., intake 2401, upstream of an intersection of a manifold lumen with a needle channel 2404, may be or may include a nozzle, e.g., a convergent nozzle, e.g., to accelerate fluid flow velocity. A second intake, e.g., intake 2402, may be positioned downstream of an intersection with a needle channel 2404. As a needle 2450 comprising a core 2000 in a needle lumen is retracted into a needle channel, e.g., is moving in proximal direction, a removal tool, e.g., a pushrod 2410, inside a needle lumen prevents a core from moving proximally, e.g., at or near an intersection of an intersection of a manifold lumen with a needle channel A removal tool, e.g., a pushrod 2410, may be stationary or moveable, e.g., moving in a direction opposite to a direction of movement of a needle. In some embodiments, proximal movement of a needle, e.g., in combination with a core held in position by a removal tool exposes a core 2000 to a fluid stream, e.g., as shown in FIG. 24B. A skin core 2000 may by sucked through the second intake, e.g., intake 2402, and/or pushed by the fluid stream generated through the first intake, e.g., intake 2401, and may be removed from the intersection (FIG. 24C).

In some embodiments, a needle hub may not include a fluid-based core clearing system, e.g., as described above. In some embodiments, during repeated operation of one or more needles, cores in a lumen of a needle may be stacked and pushed out of a lumen, e.g., out of a proximal end of a needle lumen by positive displacement. Cores exiting from a proximal end of a needle lumen may be deposited into a space, e.g., a receptacle, proximal to the needle.

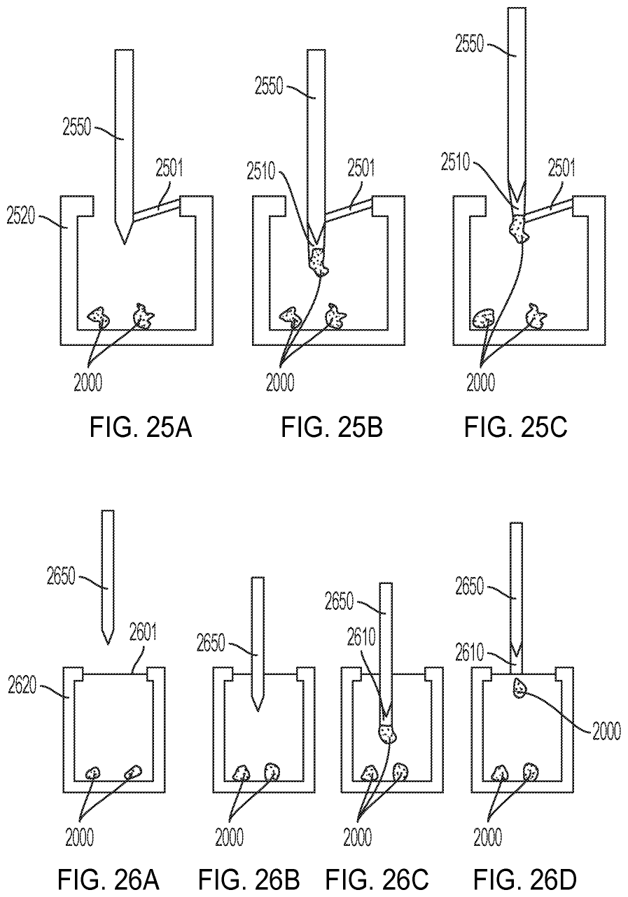

In some embodiments, one or more cores 2000 may be deposited into a receptacle (e.g., a trap 2520) by “wiping” a removal tool, e.g., a pushrod 2510, across a membrane, e.g., membrane 2501, e.g., as shown in FIGS. 25A-25C. An example needle 2550 may move relative to a removal tool, e.g., a pushrod 2510, as described herein, which may cause one or more skin cores 2000 to exit a lumen of a needle 2550, e.g., at a distal end of needle 2550 (FIG. 25A, FIG. 25B). A skin core 2000 may remain attached to a removal tool, e.g., a pushrod 2010, as it clears a distal opening of a needle (FIG. 25B). As a removal tool, e.g., a pushrod 2510, and/or a needle 2550 are retracted, a flexible membrane 2501 may contact the removal tool (e.g., a pushrod 2010) and/or skin cores 2000, removing the skin core from the removal tool (FIG. 25C). One or more “wiped” skin cores 2000 may be collected in a receptacle, e.g., receptacle 2520.

In some embodiments, one or more cores 2000 may be deposited into a receptacle after a needle punctures a membrane 2601 covering the receptacle (FIG. 26A and FIG. 26B). An example needle 2650 may move relative to a removal tool, e.g., a pushrod 2610, (e.g., by moving the needle and/or the pushrod) as described above, which may cause one or more skin cores 2000 to exit a lumen of a needle, e.g., at a distal end of needle 2650 (FIG. 26C). A skin core 2000 may remain attached to a removal tool, e.g., a pushrod 2610, as it clears a distal opening of a needle 2650. As a removal tool, e.g., a pushrod 2610, and/or needle are retracted, the flexible membrane 2601 may contact the removal tool and/or skin core 2000, removing the skin core from the removal tool (e.g., pushrod 2610) (FIG. 26D). One or more “wiped” skin cores 2000 may be collected in a receptacle, e.g., receptacle 2620.

In some embodiments, a system as described herein may include a rinsing system, e.g., including a saline flushing or rinsing system, e.g., to wash one or more needles between uses. In some embodiments, a rinsing system may include a sterile saline container that may receive one or more needles. In some embodiments, low pressure may be applied to the one or more needles drawing saline though the one or more needles, thus clearing any debris from one or more needle lumens.

In some embodiments, a lubricant may be used, e.g., to facilitate core clearing. For example, a needle tip and/or air inlet may be sprayed with or dipped in a liquid, e.g., saline to aid tissue and fluid clearing.

In some embodiments, a lumen of a needle and/or a first lumen may cylindrical. In some embodiments, a lumen of a needle and/or a first lumen may be frusto-conical, e.g., a proximal end of a lumen of a needle and/or a first lumen may have larger diameter than a distal end for contacting skin, e.g., to improve tissue transit through the lumen.

Other tissue removal tools that may be use with the technologies described herein are described in PCT Application number PCT/US2017/02475, filed Mar. 29, 2017, the disclosure of which is incorporated herein by reference in its entirety.

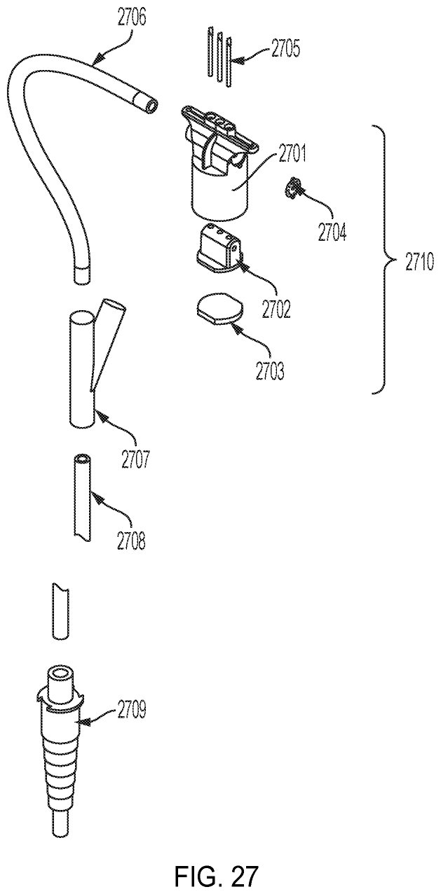

An example needle hub and core clearing system that may be used with the technologies described herein, e.g., apparatus 100, 200, or 400, is shown in FIG. 27. In some embodiments, a needle hub 2710 may include a needle hub body 2701 to hold, e.g., three example needles 2705, and a needle hub insert 2702. In some embodiments, a needle, e.g., one or more of needles 2705, may be fully or partially inserted into one or more lumens of a needle hub, e.g., needle hub body 2701. A needle may be glued, welded, or press fit into a needle hub body. In some embodiments, a needle, e.g., one or more of needles 2705, may be attached to one or more lumens of a needle hub, e.g., needle hub body 2701, without being inserted, e.g., one or more needles may be attached externally to a needle hub body, e.g., needle hub body 2701. In some embodiments, an example needle hub 2710 may include a filter 2704, e.g., to remove impurities from ambient air. In some embodiments, an example needle hub 2710 may include a secondary insert 2703, e.g., a metal (e.g., steel) insert. In some embodiments, a secondary insert 2703 may be used, e.g., to hold a needle hub insert 2702 in place. In some embodiments, a secondary insert 2703, e.g., a metal (e.g., steel) insert, may be used to verify that a needle hub 2710 is connected, e.g., securely connected, to one or more components of an apparatus as described herein, e.g., securely mounted to a z-actuator, e.g., via a needle hub mount. In some embodiments, an electrical signal or an RFID signal may be used to verify connection. In some embodiments, a secondary insert 2703 may include an RFID tag. In some embodiments, a needle hub, e.g., needle hub 2710, may be implemented as a disposable unit. In some embodiments, a needle hub, e.g., needle hub 2710, may be configured to include several components that are implemented as one or more disposable units, e.g., one or more needles and/or needle mounts. An example needle hub may include a tag or chip or other identifier, e.g., mounted on or integrated into a secondary insert 2703. In some implementations, an identifier may be used to identify a specific needle hub, e.g., to monitor usage of a needle hub as described herein.

In some embodiments, an example needle hub 2710 and core clearing system may include a fluid conduit, e.g., first tubing 2706, e.g., connected to needle hub 2710, e.g., at an end of a lumen of a needle hub body 2701, e.g., a second end of a second lumen. In some embodiments, a fluid conduit, e.g., a first tubing 2706, may be connected to a connector, e.g., a Y-connector 2707 having a first, end, a second end, and a third end. In some embodiments, a first tubing 2706 may be connected to a first end of a Y-connector 2707. In some embodiments, a Y-connector 2707 may include a second end, which may be connected to a fluid conduit (e.g., tubing) that may connect Y-connector 2707 to a spacer, e.g., a foot or frame of a vacuum spacer as described below. In some embodiments, a Y-connector 2707 may include a third end connected to a fluid conduit, e.g., second tubing 2708. In some embodiments, a second tubing 2708 may be connected to or include a connector, e.g., a stepped connector 2709. In some embodiments, a connector, e.g., stepped connector 2709, may connect a needle hub and/or core clearing system to a fluid system, e.g., a low pressure system, e.g., a vacuum pump, to induce (partial) vacuum in a system as described below.

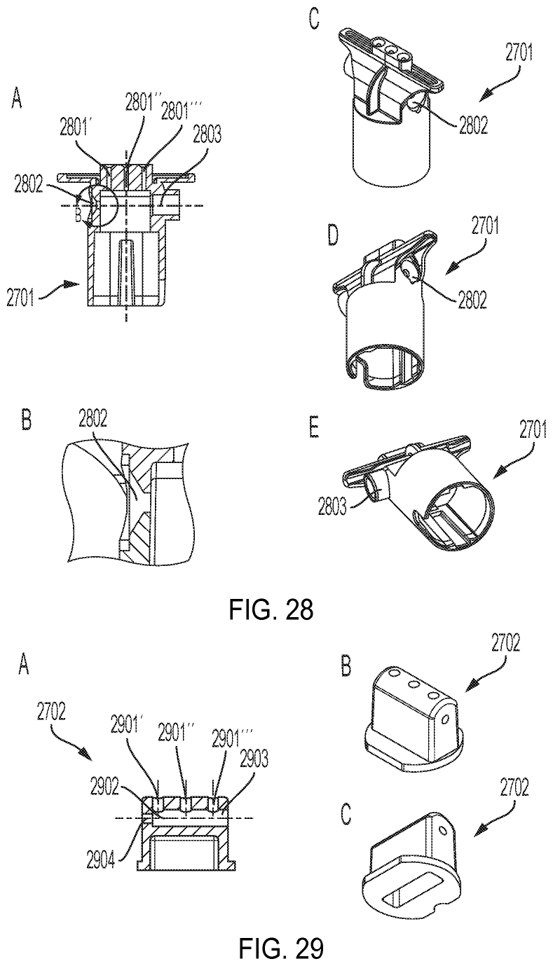

FIG. 28A shows a cross-sectional view of an example needle hub body 2701. In some embodiments, a needle hub body 2701 may include one or more first lumens, e.g., three first lumens 2801, or one or more parts thereof. In some embodiments, a needle may be fully or partially inserted in a first lumen (e.g., lumen 2801) of a needle hub body, e.g., needle hub body 2701. In some embodiments, a needle, e.g., a hollow needle having a lumen, may be attached to a needle hub body, e.g., needle hub body 2701, such that a first lumen of a needle hub body and a needle lumen are connected (e.g., end-to-end) and together form a first lumen of a needle hub, e.g., needle hub 2710, or a part thereof. In some embodiments, a needle hub body 2701, may include a fluid intake nozzle, e.g., nozzle 2802. In some embodiments, a fluid intake nozzle, e.g., nozzle 2802, of a needle hub body 2701, may constitute a part of a second lumen of a needle hub body, and/or may be located at a first end of a second lumen of a needle hub body (FIG. 28B). In some embodiments, a needle hub body, e.g., needle hub body 2701, may include a second lumen, or a part thereof, e.g., an upstream section 2803 of a second lumen of a needle hub body 2701. In some embodiments, a fluid intake nozzle and a second lumen of a needle hub body, e.g., an upstream section 2803 of a second lumen of a needle hub body 2701, may be part of a second lumen of a needle hub. FIGS. 28C-28E show perspective views of an example needle hub body 2701.

FIG. 29A shows a cross-sectional view of an example needle hub insert 2702. In some embodiments, a needle hub insert, e.g., needle hub insert 2702, may include one or more first lumens, e.g., three first lumens 2901, or one or more parts thereof. In some embodiments, a needle hub insert may be configured such that one or more first lumens of a needle hub insert 2702 line up with one or more first lumens of a needle hub body, e.g., first lumen 2801 of needle hub body 2701, when a needle hub insert is inserted in a needle hub body. In some embodiments, a lumen of a hollow needle, a first lumen of needle hub body, e.g., needle hub body 2701, and a first lumen of a needle hub insert, e.g., needle hub insert 2702, may be connected (e.g., end-to-end) and together form a first lumen of a needle hub, e.g., needle hub 2710, or a part thereof. For example, lumen 2901′ may line up with lumen 2801′, lumen 2901″ may line up with lumen 2801″, and lumen 2901′″ may line up with lumen 2801′″. In some embodiments, a lumen of a hollow needle may be inserted in a needle hub body 2701 such that a lumen of a hollow needle, a lumen of a needle hub body 2701, and a first lumen of needle hub insert 2702 are connected (e.g., end-to-end) and together form a first lumen of a needle hub 2710, or a part thereof. In some embodiments, a needle hub insert 2702 may include a second lumen. e.g., second lumen 2902, having a first end and a second end. In some embodiments, a fluid intake nozzle, e.g., nozzle 2802, a second lumen (e.g., upstream section 2803) of a needle hub body 2701 and a second lumen 2902 of a needle hub insert 2702) may align and constitute a second lumen of a needle hub 2710 or may be part of a second lumen of a needle hub 2710. In some embodiments, a needle hub insert 2702 may be configured such that when needle hub insert 2702 is inserted into a needle hub body 2701, fluid entering a needle hub through an intake nozzle (e.g., nozzle 2802) of a needle hub body 2701 may subsequently enter a second lumen 2902 of a needle hub insert 2702 through an opening at a first end of a lumen of a needle hub insert 2702. Fluid may then traverse a second lumen 2902 of a needle hub insert 2702, and exit the second lumen 2902 of a needle hub insert 2702 through an opening at a second end 2903 of a second lumen 2902 of a needle hub insert 2702. Fluid exiting a second lumen 2902 of a needle hub insert 2702 through an opening 2903 at a second end of a second lumen 2902 of a needle hub insert 2702 may enter a second lumen of a needle hub body 2701, e.g., an upstream section 2803 of a second lumen of a needle hub body 2701. In some embodiments, an opening 2903 at a second end of a second lumen 2902 of a needle hub insert 2701 has a larger cross sectional area than an opening 2904 at a first end of a second lumen 2902 of a needle hub insert. In some embodiments, a second lumen (e.g., upstream section 2803) of a needle hub body 2701, and a second lumen 2902 of a needle hub insert 2702 may constitute a second lumen of a needle hub 2710 or may be part of a second lumen of a needle hub 2710. FIG. 29B and FIG. 29C show perspective views of an example needle hub insert 2702.

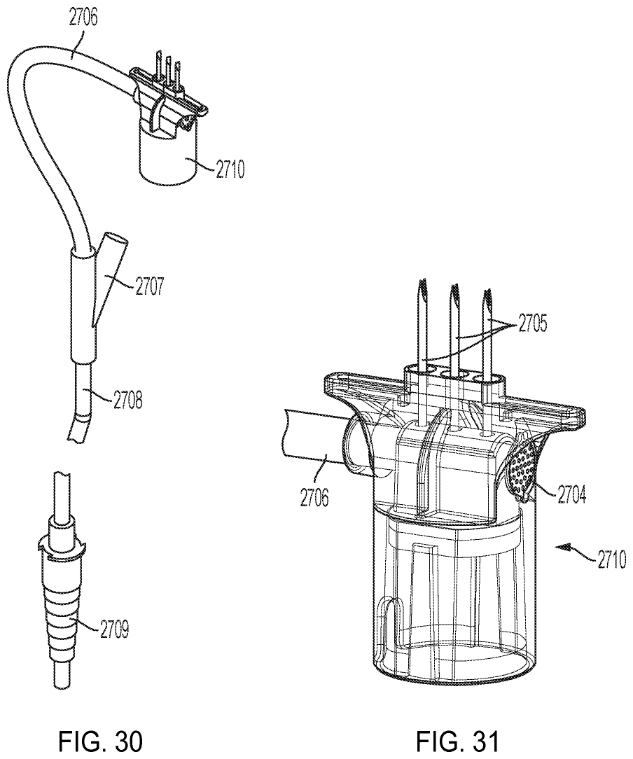

FIG. 30 shows an assembled example needle hub 2710 and core clearing system that may be used with the technologies described herein. FIG. 27 shows a semi-transparent view of an assembled example needle hub 2710 including three needles 2705.

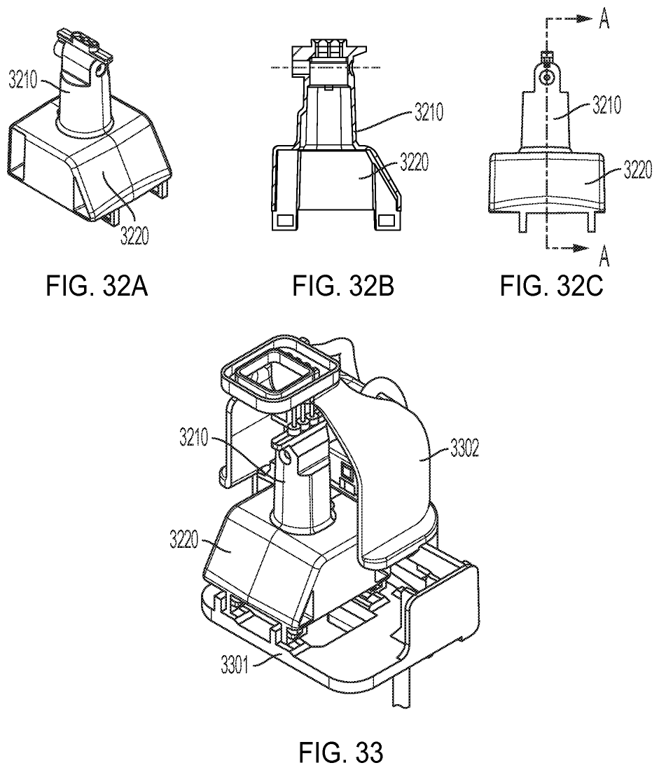

In some embodiments, a system as described herein may include technologies to prevent fluids or other substances from entering an apparatus (e.g., an apparatus 100, 200, or 400), e.g., a distal opening in a hand piece, e.g., hand piece 120, 220, or 420. In some embodiment, a needle hub may include or may be connected to a shield to prevent fluids or other substances from entering an apparatus, e.g., a distal opening in a hand piece, e.g., a hand piece 120, 220, or 420. FIG. 32A-C shows an example needle hub 3210, which may be substantially similar or the same as needle hub 2710, connected to example hub shield 3220. In some embodiments, as a needle hub, e.g., needle hub 3210, moves in the x-direction or y-direction (e.g., substantially parallel to a skin surface) and/or moves in the z-direction (e.g., substantially perpendicular to a skin surface), an example hub shield 3220 may move together with a needle hub 3210. In some embodiments, an example hub shield 3220 is sized such that a distal opening or distal end of a hand piece (e.g., a hand piece 120, 220, or 420) is covered by at least a portion of a hub shield 3220 and/or needle hub 3210. FIG. 33 shows an example needle hub 3310 and hub shield 3220 moveably mounted on an example hand piece distal end component 3301 and an example spacer 3302. Example spacers, e.g., spacer 3302, are further described below (e.g., spacers 4000 or 4100). In some embodiments, example needle hub 3310 and hub shield 3220, example hand piece distal end component 3301 and/or example spacer 3302 may be reusable. In some embodiments, example needle hub 3310 and hub shield 3220, example hand piece distal end component 3301 and/or example spacer 3302 may be disposable. In some embodiments, example hub shield 3220 maybe releasably connected to hand piece distal end component 3301. In some embodiments, e.g., where needle hub, hub shield, and or hand piece distal end component are disposable, example hub shield 3220 may be connected to hand piece distal end component 3301 during storage and/or transport (e.g., through a openable locking mechanism, e.g., a hooking mechanism), but may be released from hand piece distal end component 3301 after hand piece distal end component 3301 is connected to a hand piece, e.g., hand piece 220. Other example embodiments are discussed below.

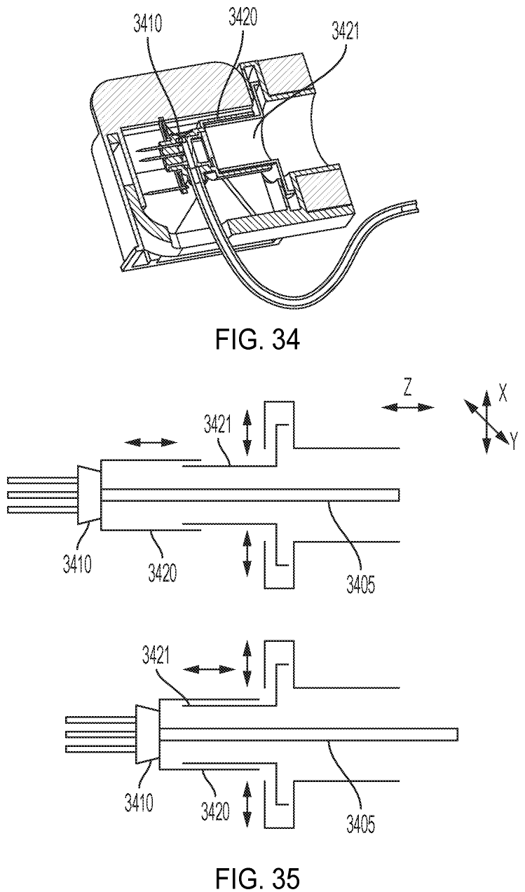

In some embodiments, technologies to prevent fluids or other substances from entering an apparatus (e.g., an apparatus 100, 200, or 400) may include multiple components. An example needle hub assembly including an example needle hub 3410, an example ingress shield 3420, and example shield receiver 3421 is shown in FIG. 34. A schematic illustrating the working principle of this embodiment is shown in FIG. 35. An example cylindrical ingress shield 3420 may be positioned proximal to one or more needles or a needle hub 3410 and may move with the needle hub, e.g., in a z-direction perpendicular to a surface of skin to be treated. Needle hub 3410 may be connected to x-, y-, and z-actuators, e.g., through push rod 3405. An example shield receiver 3421 moveable in x-y direction, e.g., parallel to a surface of skin, may be positioned inside a cylindrical shield such that a needle hub 3410 may move in all directions while maintaining overlap between shield and shield receiver. Thereby, a tortuous path may be created for any potential contaminant, thus protecting internal components of a hand piece.

FIG. 36 shows an example needle hub and core clearing system substantially as described above (e.g., as described for the embodiment in FIG. 27), configured for a single needle, e.g., needle 3605. In some embodiments, a needle hub 3610 may include a needle hub body 3601 to hold, e.g., an example needle 3605, and a needle hub insert 3602. Needle hub insert 3602 may be substantially similar or the same as needle hub insert 2702. In some embodiments, a needle, e.g., a needle 3605, may be fully or partially inserted into one or more lumens of a needle hub, e.g., needle hub body 3601. A needle may be glued, welded, or press fit into a needle hub body. In some embodiments, a needle, e.g., needle 3605, may be attached to one or more lumens of a needle hub, e.g., needle hub body 3601, without being inserted, e.g., a needles may be attached externally to a needle hub body, e.g., needle hub body 3601. In some embodiments, an example needle hub 3610 may include a filter 3604, e.g., to remove impurities from ambient air. In some embodiments, an example needle hub 3610 may include a secondary insert 3603, e.g., a metal (e.g., steel) insert. In some embodiments, a secondary insert 3603 may be used, e.g., to hold a needle hub insert 3602 in place. In some embodiments, a secondary insert 3603, e.g., a metal (e.g., steel) insert, may be used to verify that a needle hub 3610 is connected, e.g., securely connected, to one or more components of an apparatus as described herein, e.g., securely mounted to a z-actuator, e.g., via a needle hub mount. In some embodiments, an electrical signal or an RFID signal may be used to verify connection. In some embodiments, a secondary insert 3603 may include an RFID tag. In some embodiments, a needle hub, e.g., needle hub 3610, may be implemented as a disposable unit. In some embodiments, a needle hub, e.g., needle hub 3610, may be configured to include several components that are implemented as one or more disposable units, e.g., a and/or a needle mount. An example needle hub may include a tag or chip or other identifier, e.g., mounted on or integrated into a secondary insert 3603. In some implementations, an identifier may be used to identify a specific needle hub, e.g., to monitor usage of a needle hub as described below.

In some embodiments, an example needle hub 3610 and core clearing system may include a fluid conduit, e.g., first tubing 3606, e.g., connected to needle hub 3610, e.g., at an end of a lumen of a needle hub body 3601, e.g., a second end of a second lumen. In some embodiments, a fluid conduit, e.g., a first tubing 3606, may be connected to a connector, e.g., a Y-connector 3607 having a first, end, a second end, and a third end. In some embodiments, a first tubing 3606 may be connected to a first end of a Y-connector 3607. In some embodiments, a Y-connector 3607 may include a second end, which may be connected to a fluid conduit (e.g., tubing) that may connect Y-connector 3607 to a spacer, e.g., a foot or frame of a vacuum spacer as described below. In some embodiments, a Y-connector 3607 may include a third end connected to a fluid conduit, e.g., second tubing 3608. In some embodiments, a second tubing 3608 may be connected to or include a connector, e.g., a stepped connector 3609. In some embodiments, a connector, e.g., stepped connector 3609, may connect a needle hub and/or core clearing system to a fluid system, e.g., a low pressure system, e.g., a vacuum pump, to induce (partial) vacuum in a system as described below.

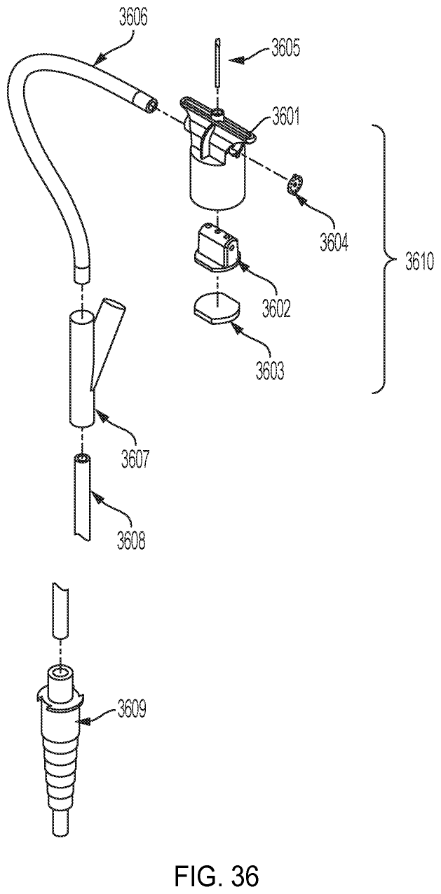

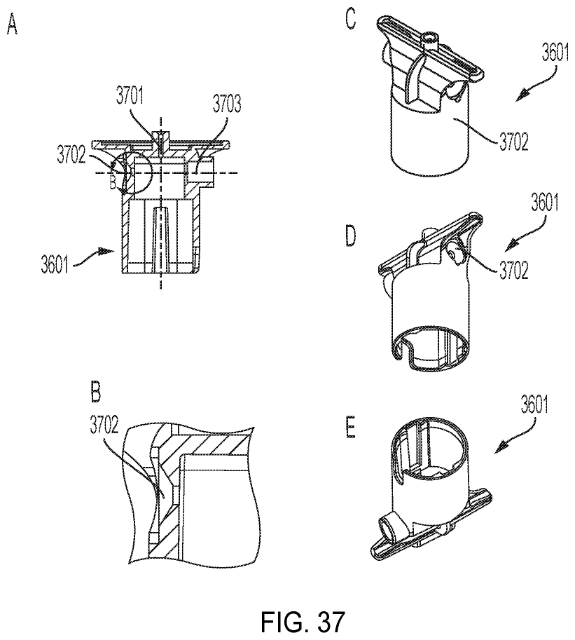

An example needle hub body 3601 to be use with an example system as shown in FIG. 36 is shown in FIGS. 37A-37E. In some embodiments, a needle hub insert as shown, e.g., 3602 or 2702 in FIG. 29 may be used in a single needle system as shown in FIG. 36. In some embodiments, a needle hub insert with a single first lumen may be used in a single needle system as shown in FIG. 36. FIG. 37A shows a cross-sectional view of an example needle hub body 3601. In some embodiments, a needle hub body 3601 may include a first lumen, e.g., first lumen 3701, or one or more parts thereof. In some embodiments, a needle may be fully or partially inserted in a first lumen of a needle hub body, e.g., needle hub body 3601. In some embodiments, a needle, e.g., a hollow needle having a lumen, may be attached to a needle hub body, e.g., needle hub body 3601, such that a first lumen 3701 of a needle hub body and a needle lumen are connected (e.g., end-to-end) and together form a first lumen of a needle hub, e.g., needle hub 3610, or a part thereof. In some embodiments, a lumen of a hollow needle, a first lumen of needle hub body, e.g., needle hub body 3601, and a first lumen of a needle hub insert, e.g., needle hub insert 3602 or 2702, may be connected (e.g., end-to-end) and together form a first lumen of a needle hub, e.g., needle hub 3610, or a part thereof. In some embodiments, a lumen of a hollow needle, a first lumen of needle hub body, e.g., needle hub body 3601, and a first lumen 2901″ of needle hub insert 2702, may be connected (e.g., end-to-end) and together form a first lumen of a needle hub, e.g., needle hub 3610, or a part thereof. In some embodiments, a needle hub body 3601 may include a fluid intake nozzle, e.g., nozzle 3702. In some embodiments, a fluid intake nozzle, e.g., nozzle 3702, of a needle hub body 3601, may constitute a part of a second lumen of a needle hub body, and/or may be located at a first end of a second lumen of a needle hub (FIG. 37B). In some embodiments, a needle hub body, e.g., needle hub body 3601, may include a second lumen, or a part thereof, e.g., an upstream section 3702 of a second lumen of a needle hub body 3601. In some embodiments, a fluid intake nozzle, e.g., nozzle 3702, and a second lumen of a needle hub body, e.g., an upstream section 3703 of a second lumen of a needle hub body 3601, may be part of a second lumen of a needle hub. FIGS. 37C-37E show perspective views of an example needle hub body 3601.

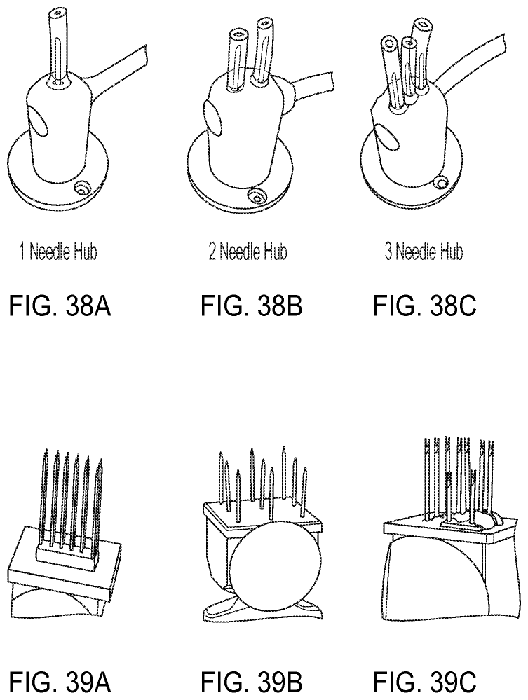

Needle hubs of any needle/lumen configuration may be used with the technologies described herein. FIG. 38A shows an example needle hub with one coring needle, FIG. 38B, shows an example needle hub with two coring needles, FIG. 38C shows a needle hub with three coring needles. Coring needles may be arranged in one or two-dimensional arrays, may be aligned or staggered, and/or may be spaced uniformly or non-uniformly. Needles of different lengths may be used in a same array. FIG. 36A shows a 6×1 needle array, FIG. 39B shows a 3×3 needle array, and FIG. 39C shows a 3×3 needle array with needles of different lengths within the same array.

Consumable Detection—Needle Hub

A needle hub may be configured or implemented as a single-use item or a reusable item. In some embodiments, a reusable needle hub may be sterilizeable and/or autoclavable (e.g., may be constructed from heat resistant materials).

In some embodiments, a needle hub as described herein (e.g., needle hub 2710 or needle hub 3610) may include a tag to identify a needle hub. In some embodiments, a tag may be or include an integrated circuit (IC) chip that may be read-only. In some embodiments, a tag may be or include a chip that may be a read-and-write chip. In some embodiments, a tag may be or include a chip that is operable to exchange data with a reader using, e.g., RF signals and may include a built-in antenna and an integrated circuit, e.g., a tag may be or include an RFID tag. In some embodiments, a tag may be or include an RFID chip mounted on or integrated into a needle hub, e.g., in or on a secondary insert (e.g., secondary insert 2703 or 3603) of a needle hub (e.g., needle hub 2710 or needle hub 3610).

In some implementations, an identifier may be used to identify a specific needle hub, e.g., to monitor usage of a needle hub as described below.

Spacer

The technologies described herein may include a system and/or apparatus that includes a spacer. In some embodiments, a spacer may be part of or connected to an apparatus as described herein (e.g., apparatus 100, 200, or 400), e.g., may be part of or attached to a hand piece (e.g., hand piece 120, 220 or 420), e.g., a hand piece shell, of an example apparatus. In some embodiments, a spacer may be used to maintain a constant distance between a base position (e.g., retracted position) of a needle and a surface of skin to be treated. In some embodiments a spacer may be adjustable or moveable, e.g., to adjust the distance between a base position of a needle (and/or a distance between a z-actuator) and a surface of skin to be treated. In some embodiments, a distance between a base position of a needle (and/or a distance between a z-actuator) and a surface of skin to be treated may be adjusted during a coring procedure. In some embodiments, a distance between a base position of a needle (and/or a distance between a z-actuator) and a surface of skin to be treated may be adjusted prior to a coring procedure and may remain constant during a coring procedure.

In some embodiments, a spacer may be or include a one or more devices to maintain a distance and/or position (e.g., a constant distance and/or position) of an apparatus relative to a skin surface to be treated during a coring procedure. In some embodiments, a spacer may be or include a one or more devices to maintain or increase tension in a skin tissue to be during treatment compared to skin not being treated and/or contacted by an apparatus described herein. In some embodiments, one or more devices to maintain a distance and/or position are different form one or more devices to maintain or increase tension in a skin tissue. In some embodiments, one or more devices to maintain a distance and/or position are the same as one or more devices to maintain or increase tension in a skin tissue. In some embodiments, one or more devices to maintain a distance and/or position and/or one or more devices to maintain or increase tension in a skin tissue may include hooks, barbs may include one or more tissue fixation implements including frames, pins, rollers, forceps, grippers, hooks, needles, barbs, and/or adhesives.

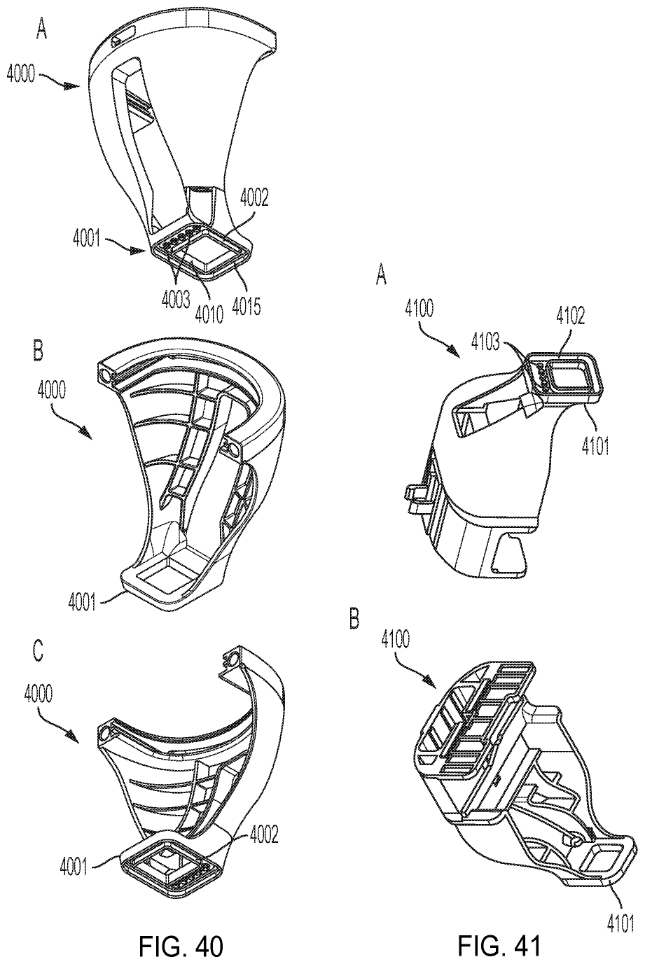

In some embodiments, a spacer may be or include a vacuum spacer. An example vacuum spacer 4000 is shown in FIGS. 40A-40C. An example vacuum spacer may include a frame 4001 to contact a surface of a skin tissue to be treated. In some embodiments, a frame 4001 of a spacer 4000 may be configured such that the frame forms a border around an area of skin to be treated, e.g., cored by one or more coring needles. An example frame of a spacer may include a base, an inner wall 4010, and an outer wall 4015, wherein the base, inner wall, and outer wall form an open channel in the frame. An example channel 4002 may be configured such that when a frame is placed on a surface of skin, the surface of the skin, the base, the inner wall 4010, and outer wall 4015 form a lumen, e.g., a frame lumen. In some embodiments, a frame 4001 may include one or more protrusions, e.g., one or more protrusions 4003, e.g., to reduce an amount of skin tissue drawn into a channel 4002.

FIG. 41A and FIG. 41B show another, similar, example spacer 4100 with (vacuum) frame 4101, example frame channel 4102, and example protrusions 4103. Example spacer 4100 may be substantially similar or the same as spacer 3302 shown in FIG. 33.

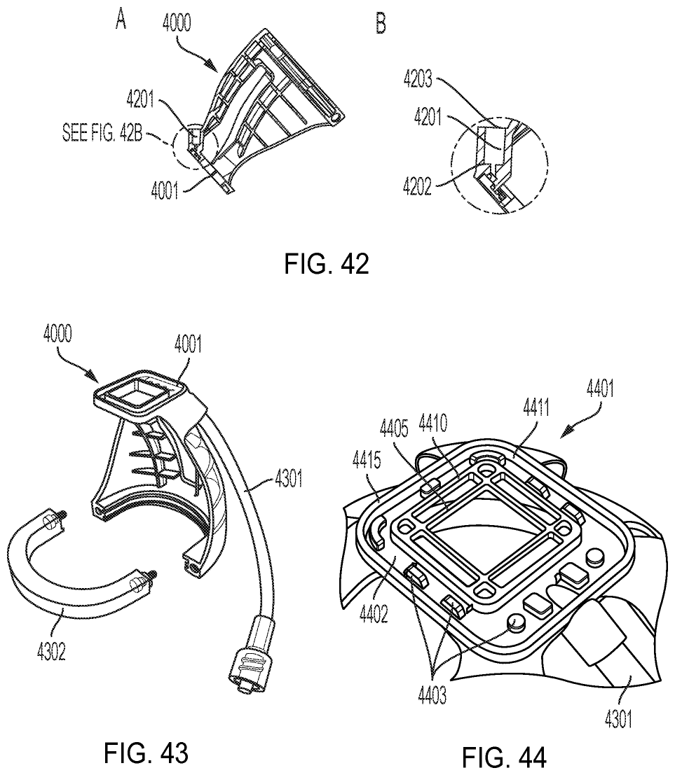

In some embodiments, a frame, e.g., frame 4001 or 4101, may be connected to a fluid conduit such that when low pressure (e.g., below atmospheric pressure) or (partial) vacuum is applied to the fluid conduit, low pressure or (partial) vacuum is established in the frame lumen (e.g., frame lumen 4002 or 4102). FIGS. 42A and 42B show a section of an example vacuum spacer frame (e.g., frame 4000) including a connection lumen 4201 having a first end 4202 and a second end 4203. In some embodiments, a first end 4202 of a connection lumen 4201 may form an opening in a frame lumen, e.g., lumen 4002. In some embodiments, a second end 4203 of a connection lumen 4201 may contact an end of a lumen of a fluid conduit. In some embodiments, a fluid conduit may be connected to a Y-connector 2707 as shown in FIG. 27 (e.g., a second end of a Y-connector) and/or a low pressure source, e.g., a vacuum pump. Low pressure or (partial) vacuum in a frame lumen may cause skin tissue to be drawn towards (e.g., sucked into) the channel.

In some embodiments, applying low pressure (e.g., below atmospheric pressure) or (partial) vacuum to a channel of a vacuum spacer frame (e g, channel 4002 or 4102) may cause a suction force to be exerted on skin tissue contacting the frame. This may cause an increase in tension in an area of skin near (e.g., surrounded by) or in contact with a vacuum spacer frame. Without wishing to be bound by theory, increased tension in skin tissue surrounded by a vacuum spacer frame under low pressure or (partial) vacuum may cause stabilization of a plane of the skin surface such that when surface penetration by a coring needle begins, movement of skin in contact with the needle in direction of needle movement during coring (“tenting”) is reduced compared to movement of skin during a similar procedure without application of a vacuum spacer frame. A coring needle may penetrate a dermis at a lower velocity and/or force than would be required in a similar procedure without application of a vacuum spacer frame to a skin surface. In some example embodiments, application of a vacuum space frame may yield more consistent/reproducible depth of penetration of a needle, e.g., in relation to a skin surface and/or a vacuum spacer frame, compared to a similar procedure without application of a vacuum spacer frame, e.g., due to reduced movement of skin. In some example embodiments, application of a vacuum spacer frame may lead to a lower depth of penetration of a needle, e.g., in relation to a skin surface and/or a vacuum spacer frame, required to achieve a similar effect compared to a similar procedure without application of a vacuum spacer frame, e.g., due to reduced movement of skin or compression of one or more skin layers. Use of a vacuum spacer frame may reduce trauma (reduce down time), enhance safety, and/or reduce chances of over-penetration. In some embodiments, a low pressure or (partial) vacuum generated in a vacuum frame may enable a user to pull skin tissue connected to the vacuum frame away from anatomical structures beneath the skin, e.g., reducing the potential for the needle to contact undesired underlying structures during actuation. In an example procedure without a vacuum spacer frame, a coring needle may push skin away in direction of needle tip movement as the needle is penetrating skin, which may necessitate a deeper penetration by a needle to reach the patient's lower dermis and adjacent fat layer, e.g., to remove a full thickness core.

FIG. 43 shows an example vacuum spacer (e.g., spacer 4000), an example fluid conduit 4301 connected to a frame 4001 of the vacuum spacer 4000, and a connection frame 4302 to connect a vacuum spacer (e.g., vacuum spacer 4000) to, e.g., a hand piece (e.g., hand piece 120, 220, or 420), e.g., a hand piece shell (e.g., hand piece 121, 221, or 421), of a coring apparatus (e.g., apparatus 100, 200, or 400). FIG. 44 shows an example vacuum spacer frame 4401 (substantially similar to frame 4000 and frame 4100) to draw skin within the frame taught, e.g., to stabilize skin during coring. In some embodiments, a vacuum spacer frame (e.g., frame 4401) may include a sub-frame (e.g., sub-frame 4405), e.g., to aid positioning of a frame and/or to provide tissue stabilization.

In some embodiments, a channel in a vacuum spacer frame (e.g., frame 4401) may include one or more protrusions (e.g., protrusions 4403), e.g., one or more structures protruding from a base (e.g., base 4411) of a channel (e.g., channel 4402) in a vacuum spacer frame, e.g., to ensure even suction pressure throughout a frame lumen, e.g., as shown in FIG. 44. When low pressure or (partial) vacuum is applied to a lumen formed by a channel in a vacuum spacer frame and a skin surface (e.g., a frame lumen), skin tissue may be drawn toward the base of the channel Skin tissue may block a first end of a connection lumen that may form an opening in a frame lumen, blocking fluid communication between the frame lumen and a fluid conduit (e.g., conduit 4301) that provides low pressure or (partial) vacuum, potentially disrupting a low-pressure connection between a vacuum spacer frame and a skin surface. One or more structures protruding from a base of a channel in a vacuum spacer frame may be configured to prevent blocking of a first end of a connection lumen by skin tissue. In some embodiments, a channel in a vacuum spacer frame includes one or more indentations, e.g., one or more cavities extending into a base of a channel in the vacuum spacer frame, e.g., to ensure even suction pressure throughout a frame lumen. In some embodiments, one or more cavities extending into a base of a channel (e.g., base 4411) in a vacuum spacer frame may be configured to prevent blocking of a first end of a connection lumen by skin tissue. In some embodiments, a channel in a vacuum spacer frame may include one or more protrusions (e.g., protrusions 4003, 4103, or 4403) and one or more indentations, e.g., to ensure even suction pressure throughout a frame lumen. In some embodiments, frame channel configurations and/or protrusion configurations and/or indentation configurations may be chosen or modified depending on tissue type and/or location to be treated. Without wishing to be bound by theory, the softer or laxer a skin tissue, the closer and/or the larger protrusion may be to prevent or impede skin tissue from entering space between a protrusion and a wall (e.g., outer wall 4415 and inner wall 4410) and/or another protrusion.

In some embodiments, a channel (e.g., channel 4002, 4102, or 4402) of a vacuum spacer frame may have a width of about 2.5 mm (e.g., a minimum distance between an inner wall (e.g., inner wall 4010 or 4410) and an outer wall (e.g., outer wall 4015 or 4415) of a frame (e.g., frame 4000, 4100, or 4401) of about 2.5 mm). In some embodiments, a channel of a vacuum spacer frame may have a depth of about 2 mm (e.g., an average distance between a base of a frame and a flat surface opposite the base and substantially in contact with an outer wall of the frame). A channel of a vacuum space frame may have any size, e.g., depending on tissue to be stabilize and/or improve access to complex anatomical areas. In some embodiments, a channel of a vacuum spacer frame may have a width (e.g., a minimum distance between an inner wall an outer wall of a frame) of about 0.5. mm, 1 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, a channel of a vacuum spacer frame may have a width (e.g., a minimum distance between an inner wall an outer wall of a frame) of between 0 and 100 mm, between 10 mm and 90 mm, between 20 mm and 80 mm, or between 30 mm and 70 mm.

In some embodiments, a channel (e.g., channel 4002, 4102 or 4402) of a vacuum spacer frame may have a depth (e.g., an average distance between a base of a frame (e.g., base 4411 of frame 4401) and a flat surface opposite the base and substantially in contact with an outer wall (e.g., outer wall 4415) of the frame) of about 0.5 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, a channel of a vacuum spacer frame may have a depth (e.g., an average distance between a base of a frame (e.g., base 4411 of frame 4401) and a flat surface opposite the base and substantially in contact with an outer wall of the frame) of between 0 and 100 mm, between 10 mm and 90 mm, between 20 mm and 80 mm, or between 30 mm and 70 mm.

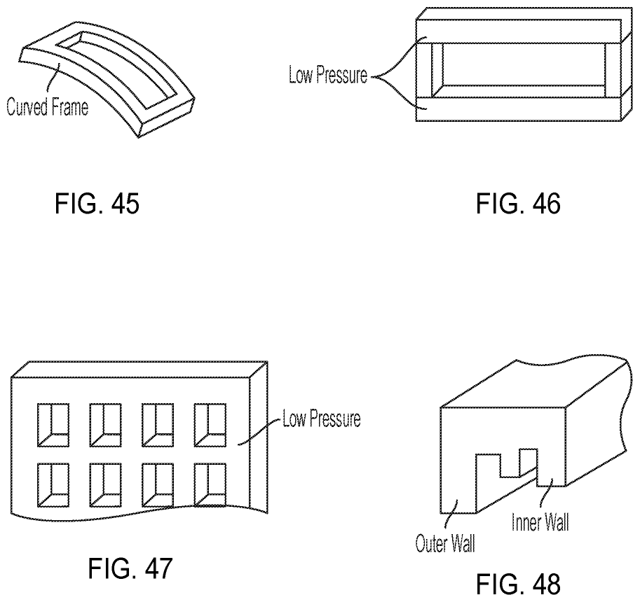

Size and/or shape of a frame of a vacuum spacer may depend on location on a body of a subject on which an apparatus may be used. Multiple variations may be employed. In some embodiments, an area of skin enclosed or surrounded by a spacer frame, e.g., surrounded by an inner wall (e.g., wall 4410 of frame 4401) a spacer frame, may have any area, e.g., an area of about 0.2 cm², 0.4 cm², 0.6 cm², 0.8 cm², 1.0 cm², 1.2 cm², 1.4 cm², 1.6 cm², 1.8 cm², 2.0 cm², 2.2 cm², 2.4 cm², 2.6 cm², 2.8 cm², 3.0 cm², 3.5 cm², 4.0 cm², 4.5 cm², 5.0 cm², 5.5 cm², 6.0 cm², 6.5 cm², 7.0 cm², 7.5 cm², 8.0 cm², 8.5 cm², 9.0 cm², 9.5 cm², 10 cm², 15 cm², or 20 cm². In some embodiments, a frame of a vacuum spacer may be curved or contoured, e.g., dependent on curvature of a tissue surface to be treated (see, e.g., FIG. 45). A contoured frame may improve contact between a frame and a skin surface compared to a flat frame. In some embodiments, a frame of a spacer, e.g., a vacuum spacer may include a sub-frame or other structure, e.g., between inner walls of a frame, e.g., as shown in FIG. 44. In some embodiments, a sub-frame (e.g., a grid) or other structure may be used to further stabilize tissue or for alignment of an apparatus.

In some embodiments, a frame of a vacuum spacer may include non-contiguous vacuum channel sections, e.g., two longer channels on opposite sides of a frame (e.g., in a rectangular frame), or non-orthogonal sections. In an example embodiment (FIG. 46), a frame includes two separate (parallel) vacuum frame elements connected by frame elements that do not conduct low pressure or (partial) vacuum. In an example embodiment (FIG. 47), a frame includes a grid of vacuum frame elements. Frame elements may be straight or curved, may be orthogonal to each other or at different angles to each other. Inner and outer channel walls are may have the same height. In some embodiments, an inner wall (e.g. inner wall 4410 of frame 4401) may have a lower height than an outer wall (e.g. outer wall 4415 of frame 4401), e.g., as shown in FIG. 48. In some embodiments, an inner wall may have a greater height than an outer wall. Varying configurations may increase or decrease an amount of stretch induced by a frame of a vacuum spacer.

The pressure in a system, e.g., in a frame lumen of a vacuum spacer, may range from approximately full vacuum (0 kPa) to approximately 50 kPa, e.g, a pressure may be between about 0 kPa and ambient atmospheric pressure, 0 kPa and 100 kPa, 5 kPa and 90 kPa, 10 kPa and 80 kPa, 15 kPa and 70 kPa, 20 kPa and 65 kPa, 25 kPa and 60 kPa, or 30 kPa and 50 kPa. In some embodiments, pressure may be kept constant during a coring procedure or may be adjusted. In some embodiments, pressure may be monitored, e.g., by measuring fluid flow rate and/or pressure. Tissue properties may be monitored, e.g., to monitor underlying tissue behavior. In some embodiments, a frame may include or be connected to one or more sensors, e.g., pressure sensors, electrical sensors, optical sensors, or cameras.

In some embodiments, a spacer may include a pressure switch, e.g., to control actuation of a z-actuator. Once a vacuum spacer of an apparatus (e.g., apparatus 100, 200, or 400) is connected to a skin surface of a subject and low pressure or (partial) vacuum is applied to a frame of a vacuum space, a user may move (e.g., gently pull up) the apparatus away from the skin surface, e.g., in a direction away from and substantially perpendicular to the skin surface. During movement of an apparatus, contact between apparatus and skin may be maintained. During movement, skin connected to an apparatus may be lifted away and/or detached from underlying tissue. During a coring procedure, a needle entering a skin tissue that has been lifted as described may be prevented from contacting tissue below a dermal layer and/or subcutaneous fat layer, even if a needle may over-penetrate a skin layer, e.g., due to an improper coring depth setting for a z-actuator.

In some embodiments, a system and/or apparatus as described herein may include a pressure switch, e.g., to prevent a z-actuator from moving unless an apparatus attached to skin tissue has been moved (e.g., pulled up) as described above. In some embodiments, a digital control system used with systems and apparatuses as described herein may include a pressure switch that may be connected to a sensor to detect a position of an apparatus relative to a skin surface and/or tissue underlying skin. In some embodiments, when a frame is placed on a skin surface and a low pressure or (partial) vacuum is applied to the frame, a switch is in a “no-go” position. After an apparatus and/or frame, while the frame is in contact with the skin surface after a low pressure or (partial) vacuum is applied to the frame, is moved in a direction that is substantially perpendicular to and away from the surface of the skin, the switch is in a “go” position. A switch may be actuated (e.g., mechanically or electrically) through a signal from a sensor that continuously senses a contact pressure between a frame and skin in contact with the frame or skin or skin tissue immediately adjacent thereto (e.g., skin tissue less than 20 mm, 15 mm, 10 mm, or 5 mm apart from an outer wall of a frame). Moving (e.g., pulling up) an apparatus may reduce contact pressure. In some embodiments, when contact pressure is reduced below a threshold, a switch may move from a “no-go” to a “go” position. When a switch is in the no-go position, a needle hub and/or z-actuator is prevented (e.g., by a digital control system) from moving along a z-axis in a direction substantially perpendicular to a surface of the skin tissue and substantially parallel to a longitudinal axis of the at least one hollow needle. When a switch is in the go position, a needle hub and/or z-actuator is moveable along the z-axis.

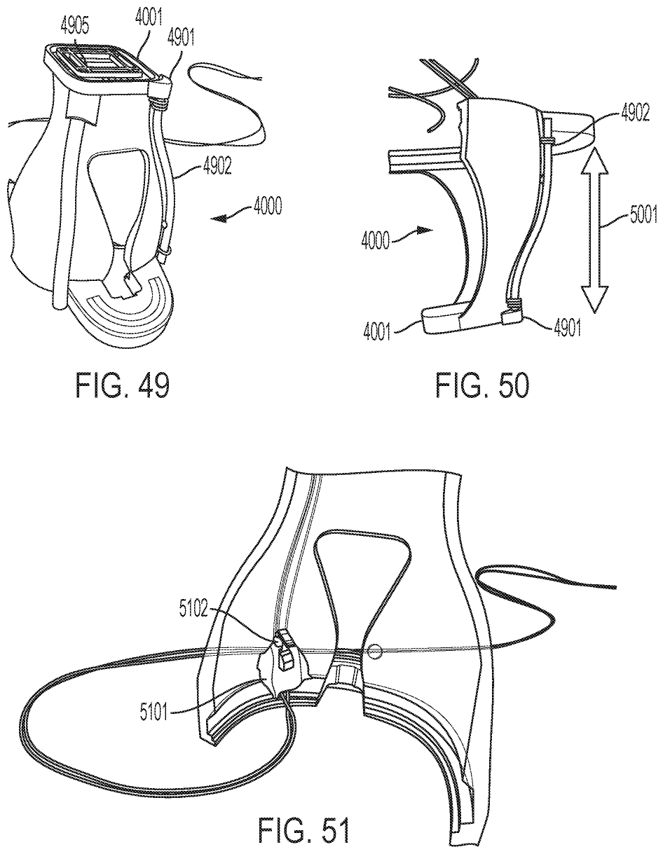

In some embodiments, an example pressure switch arrangement includes a pressure foot, including, e.g., a pressure foot tab 4901 adjacent to a frame (e.g., a frame 4001, 4101, 4401) of a vacuum spacer (e.g., a spacer 4000 or 4100), e.g., as shown in FIG. 49. A pressure foot may include a spring (connected to a switch, not shown) and/or a pushrod (e.g., pushrod 4902), which may push the pressure foot tab 4901 distally (e.g., toward a skin surface). A pressure foot pushrod may be spring-loaded, e.g., to extend a pressure foot tab distally (e.g., toward a skin surface) and distally to a frame/skin surface contact plane, e.g., at least partially beyond a skin contact end 4905 (see FIG. 50, arrow 5001 indicating directing motion of pushrod 4902). A go/no-go switch (e.g., switch 5101) may be located on or near a proximal end of a pushrod 4902 and may be connected to said pushrod 4902, e.g., at connection 5102 (see FIG. 51). When a spacer frame (e.g., a frame 4001, 4101, 4401) is placed on a skin surface and/or low pressure or vacuum is established in a frame, the pressure foot tab 4901 and the pushrod 4902 may be pushed proximally. Proximal movement of a pushrod moves a switch (e.g., switch 5101) to a retracted or “no-go” position. A switch may be connected to a digital control unit as described herein, which may be programmed such that when a no-go signal is received from the switch (e.g., switch 5101), coring (e.g., z-actuation of a needle hub) is prevented. When a spacer frame is (e.g., a frame 4001, 4101 or 4401) connected to skin tissue moved proximally (e.g., pulled up), a pressure foot tab may extend distally, e.g., via spring pressure, thereby allowing or causing the pushrod (e.g., 4902) to move distally and move the switch (e.g., switch 5101) into a “go” position. A digital control unit as described herein may be programmed such that when a go signal is received from the switch (e.g., switch 5101), coring (e.g., z-actuation of a needle hub) is permitted, e.g., a z-actuator is rendered moveable.

A spacer, e.g., a vacuum spacer as described herein, may provide an enhancement of safety wherein untargeted deeper tissues remain out of reach of a needle tip. Should a user push an apparatus down (distally) toward a skin surface, a switch may be moved to (or may remain in) a “no go” or treatment inhibited position. If a user pulls up and away from a skin surface, a pushrod may extend toward the distal end of a spacer frame moving a switch to the “go” or treatment enabled position. An apparatus with a pressure switch may be used as a technique training aid and/or may be used to teach the proper “pull up” technique.

In some embodiments, a spacer, e.g., a vacuum spacer, as described herein may include one or more tissue fixation implements including hooks, needles, barbs, and/or adhesives, e.g., to temporarily attach skin tissue to a frame. In some embodiments, an apparatus as described herein (e.g., apparatus 100, 200, or 400) may be used for treatment of facial tissue in combination with other implements. In some embodiments, application of a tongue depressor may help induce tension in tissue, e.g., in the face and/or neck, which may be beneficial for a coring procedure. After treatment, a cold (e.g., frozen) towel may be applied to cored tissue, e.g., to improve healing and/or increase tension in skin, e.g., to improve further treatment.

Accessories

Vacuum Pump System

In some embodiments, a system and/or apparatus (e.g., apparatus 100, 200, or 400) as described herein may include one or more low pressure and/or (partial) vacuum generation systems, e.g., to provide low pressure or (partial) vacuum for core clearing from a needle hub and/or to provide low pressure or (partial) vacuum, e.g., suction, to a spacer frame and/or needle hub. In some embodiments, a system or apparatus as described herein may include a low pressure or (partial) vacuum system that employs a single pump, a regulator, a control valve, and/or an inlet filter. In some embodiments, a system or apparatus as described herein may include a low pressure or (partial) vacuum system that employs a multiple (e.g., two, three, or four) pumps, regulators, control valves, and/or inlet filters.

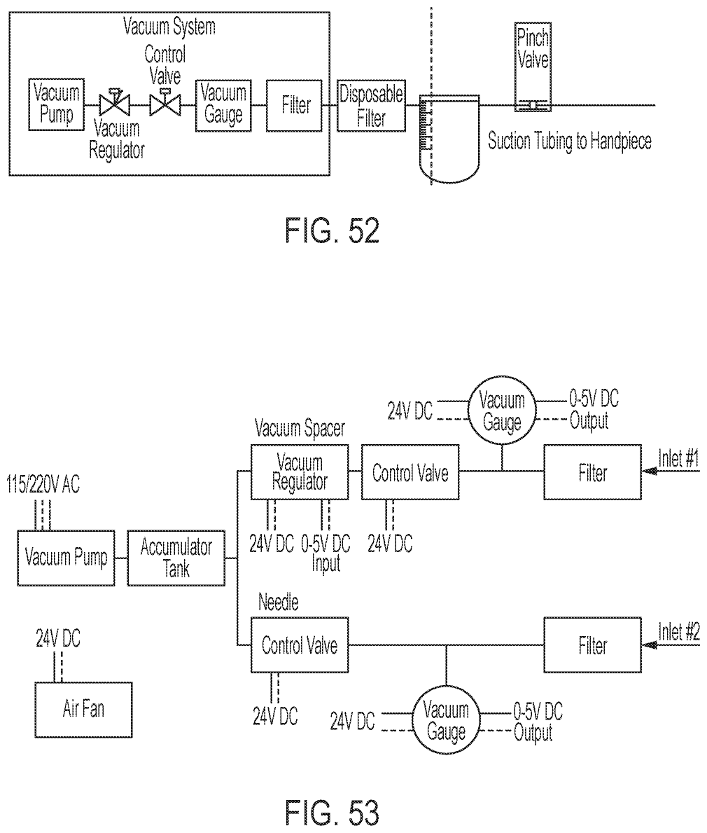

In some embodiments, a pressure conduit, e.g., tubing, connecting an element of a low pressure or (partial) vacuum system, e.g., a pump, to an apparatus, e.g., a hand piece, may be disposable. In some embodiments, a low pressure or (partial) vacuum system may include one or more filters, e.g., an air inlet filter to remove debris from ambient air while air is drawn into a system or apparatus, or one or more filters between a needle hub and a pump, e.g., to protect a pump from debris and/or contamination. In some embodiments, a low pressure or (partial) vacuum system may include one or more traps, e.g., a fluid trap and/or a skin core collection trap upstream of a needle hub. In some embodiments, a low pressure or (partial) vacuum system may include a connection conduit to connect a vacuum spacer, e.g., a spacer frame, and a needle hub. In some embodiments, a valve, e.g., an electronic pinch valve, may be used to control flow rate and/or pressure (e.g., suction) in a conduit, e.g., by collapsing one or more sections of tubing. Pressure may also be controlled using other types of valves. In some embodiments, one or more solenoid valve may be used for pressure control. In some embodiments, a low pressure or (partial) vacuum system may include an internal pressure accumulator to improve system response. A diagram of an example low pressure or (partial) vacuum system is shown in FIG. 52.

In some embodiments, pressure in a low pressure or (partial) vacuum system may be controlled through a manual valve including a vent or opening (e.g., a vent or opening in a conduit), to ambient air. An example vent or opening to ambient may be closed when low pressure or (partial) vacuum, e.g., suction, is desired. An example vent or opening may be closed by a valve or by covering a vent or opening by a finger of a user.

In some embodiments, tissue stabilization, e.g., using a vacuum spacer, may require application of a fluid pressure to a spacer, e.g., a spacer frame, that is different from a fluid pressure required for core clearing from a needle hub. In some embodiments, pressure in different locations or components of a low pressure or (partial) vacuum system may be controlled, e.g., by restricting flowrates through one or more components or compartments of a low pressure or (partial) vacuum system. In some embodiments, flow rates may be controlled, e.g., restricted, by orifices integrated in a needle hub or vacuum spacer, or in one or more conduits upstream of a needle hub or vacuum spacer. In some embodiments, flow restriction may be achieved or controlled using by electronic valves, by creating restrictive flow paths in a needle hub or vacuum spacer, or by creating restrictive flow paths in one or more conduits upstream of a needle hub or vacuum spacer. A diagram of an example low pressure or (partial) vacuum system with a subsystem for a needle hub and a subsystem for a vacuum spacer is shown in FIG. 53.

In some embodiments, a system may include two independent low pressure or (partial) vacuum systems, one connected to a needle hub for core clearing, the other connected to a vacuum spacer frame for attachment of a vacuum spacer frame to skin tissue. In some embodiments, each independent (partial) vacuum system or apparatus as described herein may include a low pressure or (partial) vacuum system that employs multiple (e.g., two, three, or four) pumps, regulators, control valves, and/or inlet filters.

In some embodiments, a low pressure or (partial) vacuum system may include one or more pressure gauges and/or one or more flow meters to monitor pressure in a low pressure or (partial) vacuum system or components thereof, e.g., continuously or sporadically. In some embodiments, a low pressure or (partial) vacuum system may include or be connected to a digital processing unit for active control and monitoring of suction function and/or performance in a subsystem for core clearing from a needle hub and/or for active control and monitoring of suction function and/or performance in a subsystem for attachment of a vacuum spacer frame to skin tissue. In some embodiments, a low pressure or (partial) vacuum system may continuously adjust suction force for each function.

Positioning and Alignment

In some embodiments, a system or apparatus (e.g., a hand piece, e.g., hand piece 120, 220, or 420) as described herein may include a translation mechanism to drive an apparatus across the skin (e.g., x- and y-translation). In some embodiments, a translation mechanism may include, e.g., driving wheels or rods. In some embodiments, a translation mechanism may permit automatic or manual translation of an apparatus across the skin. Translating components (e.g., wheels) may be disposed in or on the apparatus or may be disposed external to the apparatus, e.g., disposed in or on a hand piece or be disposed external to the hand piece. In some embodiments, a translating mechanism may be activated by an activator, such as a button, key, toggle, switch, screw, cursor, dial, spin-wheel, or other component, and/or may be digitally controlled using a digital processing unit and a user interface.

In some embodiments, a system or apparatus (e.g., a hand piece, e.g., hand piece 120, 220, or 420) as described herein may include a position detection device or system, such as an optical tracking system. In some embodiments, a position detection system may be or include a camera, an infrared sensor, a photodiode, an LED, and/or a detector and may assist in tracking movement of an apparatus in relation to a subject or a treatment area. An optical tracking mechanism may facilitate placement of a hollow needle on a skin surface in the instance of manual translation of the apparatus across the skin. In some embodiments, control electronics for a position detection mechanism may be disposed within the apparatus or external to the apparatus, e.g., integrated into a digital processing unit as described herein. In some embodiments, a position detection mechanism may monitor a distance between a previous needle insertion and the current apparatus position, and may send a signal to the control electronics to actuate the skin penetration mechanism when the apparatus has reached a desired position (e.g., a position at a defined distance from the position where the needles were last inserted). Desired distances and/or positions may be controlled at a user interface in communication with a digital processing unit.

In some embodiments, a system or apparatus as described herein may also include a guide or template to facilitate the positioning (e.g., alignment) of an apparatus and/or of a needle hub and/or of one or more hollow needles of the apparatus. In some embodiments, a guide or template may include one or more holes or openings that provide a pre-set array pattern (e.g., as described further herein) for one or more hollow needles of an apparatus to follow. A guide or template may be used alone or in combination with a position detection mechanism. In some embodiments, a hollow needle may be translated by x- and/or y-actuators to move across a guide or template and follow an array pattern set by the guide or the template to remove skin tissue portions at the holes or openings in the guide or template.

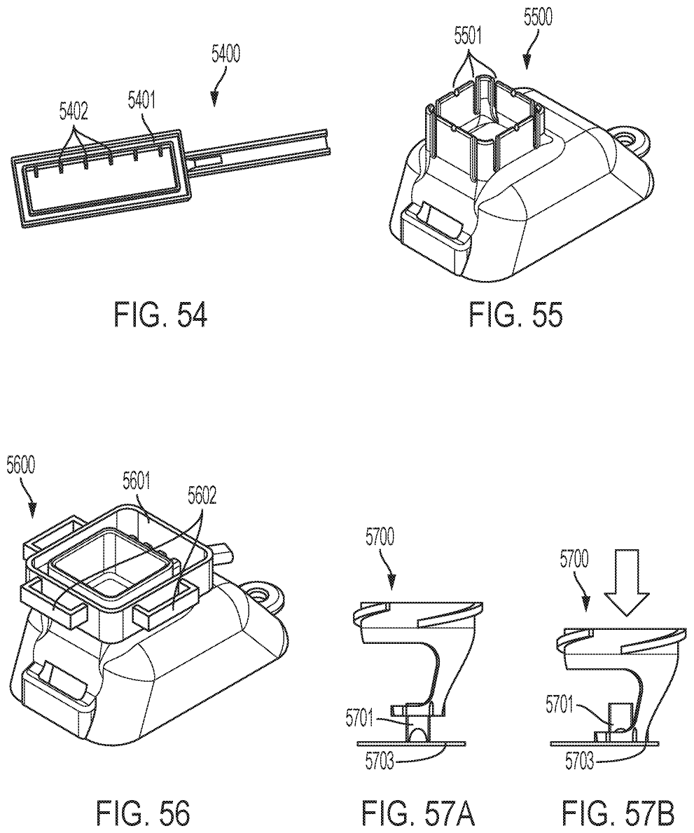

In some embodiments, a system or apparatus (e.g., apparatus 100, 200, or 400) may be positioned and/or aligned using an alignment frame. In some embodiments, a distal part of an apparatus, e.g., a spacer frame (e.g., a frame of a vacuum spacer as described above, e.g., frame 4001, 4101, or 4401), may be placed in, on, or around an alignment frame, e.g., along markings on an alignment frame (e.g., visual markers, protrusions, or magnets), to align an apparatus on a surface to be treated. In some embodiments, markers on an alignment frame may include protrusions or indentations in the alignment frame. In some embodiments, an alignment frame may be connected to a low pressure or (partial) vacuum system, e.g., as described herein, e.g., to stabilize underlying tissue as described herein with regards to a vacuum spacer frame. FIG. 54 shows an example vacuum alignment frame 5400 including a low pressure or (partial) vacuum channel 5401 and protrusions 5402 on an inner wall of the frame. In some embodiments, a vacuum alignment frame may be used in combination with a vacuum spacer frame or in combination with a coring apparatus without a vacuum spacer. In some embodiments, a vacuum alignment frame may allow re-application of an apparatus during a procedure (e.g., multiple coring cycles to cover an area larger than an area enclosed by a spacer frame of an apparatus) without disrupting tension in skin induced by (partial) vacuum or low pressure. FIG. 55 shows an example distal end component 5500 of an apparatus (e.g., attached to or part of a distal end of a hand piece, e.g., a hand piece 120, 220, or 420), e.g., to be used with an alignment frame 5400 as shown in FIG. 54. Protrusions 5402 on a (vacuum) alignment frame (e.g., frame 5400) may slot into one or more indentations (e.g., indentations 5501) of a distal end component 5501 during coring, maintaining a desired position of an apparatus. After completion of a coring cycle, an apparatus may be moved to a next position along a (vacuum) alignment frame. In some embodiments, a distal end component of an apparatus may be implemented as a vacuum spacer frame end 5600, e.g., as shown in FIG. 56. In some embodiments, a vacuum frame end 5600 (similar to vacuum frames described above e.g., frame 4001) comprising a channel 5601 may include, e.g., on one or more outer walls, one or more features 5602 that can be used to line up visually an apparatus with, e.g., a row of holes previously created.

In some embodiments, a spacer or a component thereof, e.g., a frame of a spacer as described above (e.g., frame 4001, 4101, or 4401), may include a moveable alignment element, e.g., as shown for example spacer 5700 in FIG. 57A and FIG. 57B. In FIG. 57A, a moveable alignment element 5702 is extended toward a surface 5703 of skin to be treated. In some embodiments, a moveable alignment 5702 element may be made of a transparent material and may be aligned visually prior to coring, e.g., aligned along a row of previously cored holes or aligned along an anatomical feature. FIG. 57B shows a moveable alignment element 5702 in a retracted position as an apparatus is moved toward a skin surface such that a spacer frame of spacer 5700 contacts the skin surface 5703. In some embodiments, a moveable alignment element may include or may be connected to a switch connected to a control unit that is programmed such that actuation of the switch may cause a z-actuation to be prevented when a moveable alignment element is in an extended position.

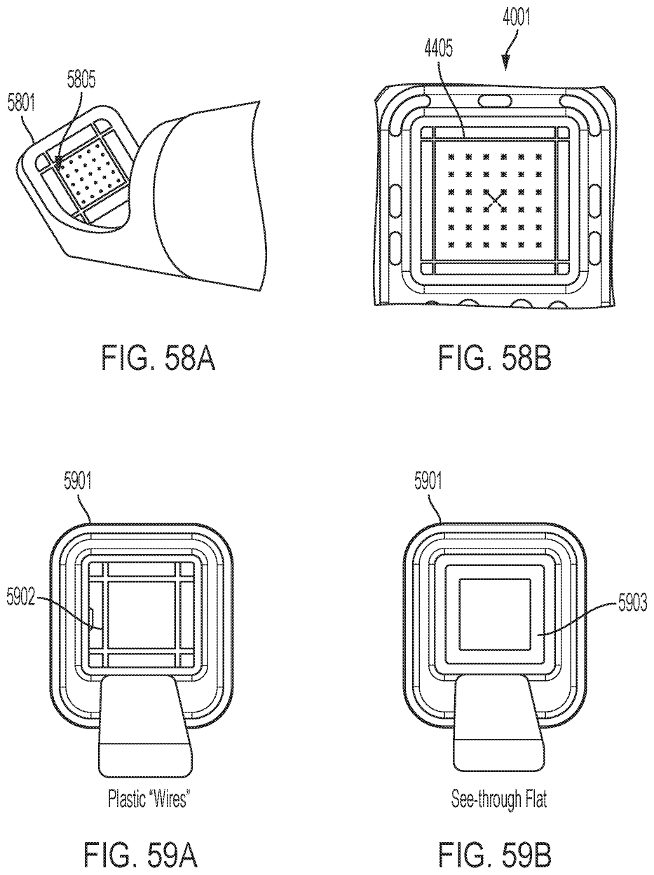

In some embodiments, a spacer frame, e.g., a vacuum spacer frame (e.g. frame 4401, e.g., as shown in FIG. 44), may include one or more inner alignment elements. In some embodiments, a spacer frame may include a sub-frame, e.g., sub-frame 4405 as shown in FIG. 44, which can be used to align one or more sub-frame elements to a row of previously cored holes. An example alignment of an example spacer frame 5801 substantially similar to frames described above using a sub-frame 5805 is shown in FIG. 58A. Bars of a spacer frame may be aligned with previously produced cores. Another example alignment of an example vacuum spacer frame using a sub-frame is shown in FIG. 58B. In some embodiments, a spacer frame may include one or more inner alignment elements in form of a grid of wires (e.g., wires 5902 as shown in a frame 5901 in FIG. 59A) or in form of a transparent element (e.g., transparent element 5903 as shown in a frame 5901 in FIG. 59B).

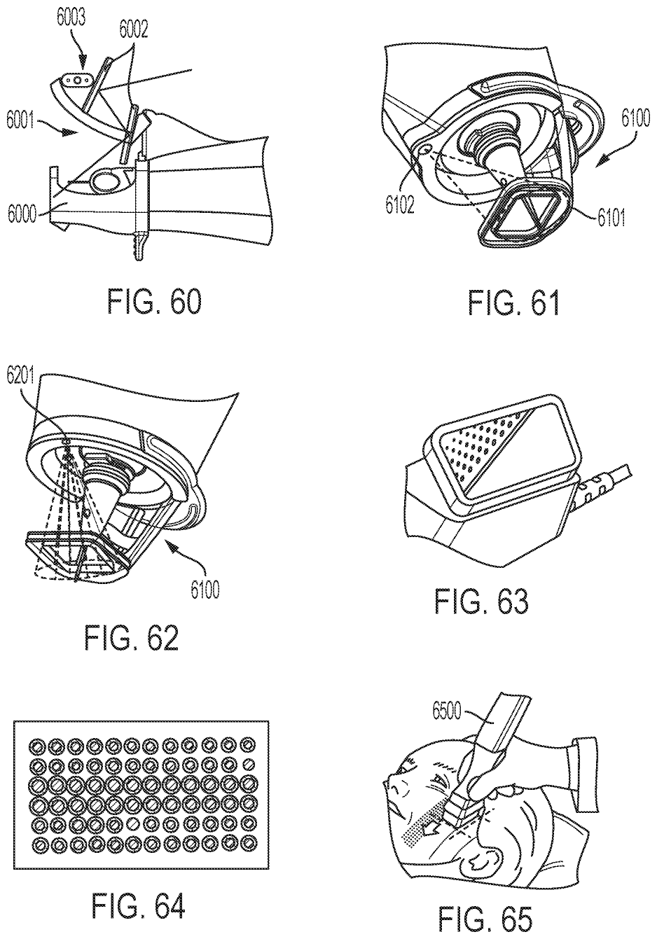

Optical technologies or devices may be used, e.g., to visually inspect a region of skin during coring or to align an apparatus, e.g., an apparatus 100, 200, or 400. In some embodiments, a spacer, e.g., a vacuum spacer including a frame, may be configured (e.g., sized) such that a region of skin being treated (e.g., cored) remains visible during a procedure. In some embodiments, a spacer may include one or more structures that create a line of sight from a side of an apparatus and/or from a position proximal to an apparatus (e.g., a hand piece, e.g., hand piece 120, 220, or 420). In some embodiments, a spacer and/or spacer frame may be made from a transparent, semi-transparent and/or translucent material. In some embodiments, a spacer may include a mirror assembly, e.g., may include a mirror connected to a ball joint to adjust positioning and line of sight. An example spacer 6000 in combination with a mirror assembly 6001 is shown in FIG. 60. One or more mirrors 6002 may be moveably mounted, e.g., on a ball joint 6003. In some embodiments, a mirror in a mirror assembly may be a concave or convex mirror, e.g., to provide visual magnification. In some embodiments, an apparatus may include or may be connected to a camera connected to a display screen. In some embodiments, an apparatus may include a camera (e.g., camera 6102) to visualize a skin region (e.g., in an example spacer frame 6101 of an example spacer 6100) during coring, e.g., as shown in FIG. 61. In some embodiments, an apparatus as described herein may include one or more sources of illumination, e.g., to aid positioning or monitoring of a treatment region. In some embodiments, a source of illumination may include a light emitting diode (LED) and/or a fiber optic cable connected to a light emitter. An example embodiment of an apparatus including a light source 6201 is shown in FIG. 62. In some embodiments, cross-polarization of light and/or specific wavelength of light may be used, e.g., to optimize skin contrast, reduce glare and/or undesired reflections, and/or improve depth perception. Light wavelength and/or light intensity may be adjusted, e.g., to improve visibility of particular structures or tissues, e.g., cored holes.

Optical devices and technologies may be used to align an apparatus (e.g., apparatus 100, 200, or 400), including, e.g., light projection devices. In some embodiments, light projection devices may be used to project cross-hairs on a skin region, aiding visual alignment of an apparatus. Light projection technologies that may be used include light emitting diodes (LEDs), lasers, and/or other light emitter that may be used for unaided visual inspection of may be used with digital light processing techniques. In some embodiments, an apparatus as described herein may be aligned using direct visual inspection and/or using a vision system, e.g., using a camera and a display. In some embodiments, a vision system may be configured to provide a display of an overlay of a desired position over a previously cored region, e.g., as shown in FIG. 63. In some embodiments, image processing systems and methods (e.g., implemented on a digital processing unit using data captured by an imaging system on an apparatus as described herein) may be used to guide a user, e.g., by analyzing an already cored region, and may provide a user with guidance as to placement of an apparatus to core a next region. An image processing system may also be used to evaluate a coring procedure (e.g., in real time), e.g., to determine unsuccessful coring. An example output of an image processing system indicating complete core removal (circles) is shown in FIG. 64. Incomplete core removal may be indicated by an absence of a circle in a cored region, or vice versa. In some embodiments, an apparatus as described herein may include one or more position tracking mechanisms, e.g., rollers, trackballs, and/or lasers, that can be used to track movement of an apparatus over a skin surface (see, e.g., FIG. 65 illustrating movement of an example apparatus 6500 over a subject's skin). In some embodiments, an apparatus may include a ball in contact with a skin surface and an electromechanical device to capture movement data of the ball as the apparatus is moved over a skin surface. Ball movement data may be used to track position of an apparatus. Position information may be processed (e.g., by a digital processing unit) and displayed, e.g., on a screen, e.g., showing an overlay over a skin region image.

Ingress Shield on Hand Piece

An apparatus as described herein (e.g., apparatus 100, 200, or 400) may include single use components and/or re-usable components. In some embodiments a needle hub may be a single-use component that is discarded, e.g., after completion of a treatment procedure. In some embodiments, one or more components of an apparatus, e.g., components encased by a hand piece shell (e.g., hand piece shell 121, 221, or 421), may be re-usable. In some embodiments, a hand piece shell may be configured to be cleaned and or sterilized. In some embodiments, a hand piece shell may be cleaned by wiping, e.g., using ethanol or bleach. In some embodiments, a hand piece shell may be covered with a disposable drape during operation.

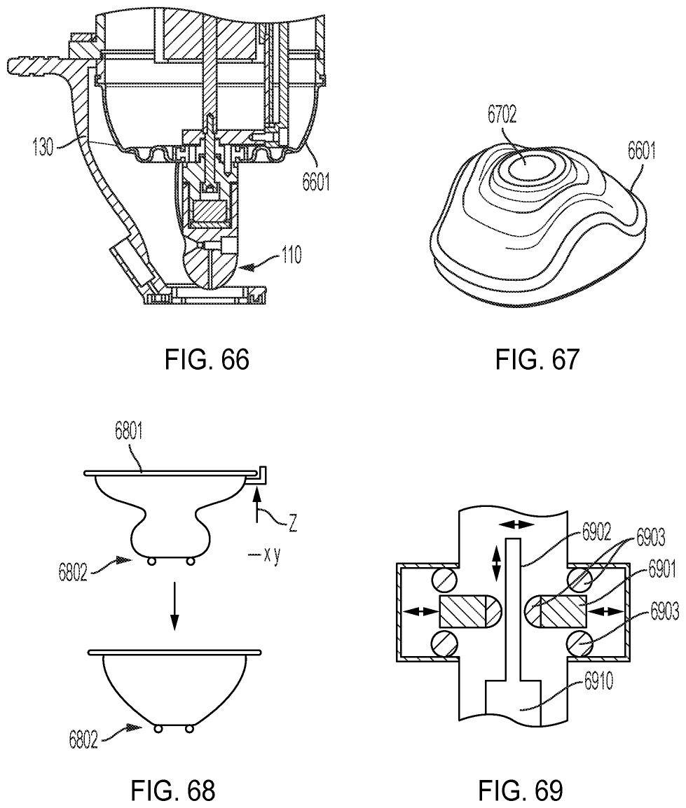

As mentioned above, in some embodiments, a system as described herein may include technologies to prevent fluids or other substances from entering an apparatus, e.g., a distal opening in a hand piece, e.g., hand piece 120, 220, or 420. In some embodiments, a hand piece ingress shield (e.g., hand piece ingress shield 6601 may be mounted on or near a distal end of an apparatus (e.g., apparatus 100) or on a distal end a of a hand piece (e.g., a hand piece 120), e.g., as shown in FIG. 66. A hand piece ingress shield 6601 may be configured such that a needle hub (e.g., needle hub 110) can move in a x-, y-, and/or z-direction while protecting an interior of a hand piece, e.g., components encased by a hand piece shell. In some embodiments, a hand piece ingress shield (e.g., a hand piece ingress shield 6601) may be or may include a flexible diaphragm including a needle hub orifice configured to provide a seal with a needle hub, e.g., diaphragm 6701, as shown in FIG. 67. A diaphragm may be made of a polymer or other flexible material, e.g., to allow for movement of a needle hub in an x- and/or y-direction, and may be re-usable or disposable. In some embodiments, a diaphragm may be mounted on a hand piece, e.g., a hand piece shell (e.g., hand piece shell 121), and may form a contact seal with a needle hub such that the needle hub may slide freely within a needle hub orifice, e.g., needle hub orifice 6702, while maintaining a seal. In some embodiments, a diaphragm, e.g., diaphragm 6801 as illustrated in FIG. 68, may be connected to a needle hub at a needle hub orifice, e.g., using a clamp 6802 or similar. A clamped diaphragm may be sufficiently flexible to allow needle hub movement in the z-direction (e.g., perpendicular to a skin surface) and/or in the x-/y-direction (e.g., parallel to a skin surface). As a needle hub is actuated, e.g., in a z-direction (e.g., perpendicular to a skin surface to be treated), the needle hub orifice moves together with the needle hub, e.g., in a z-direction, e.g., as shown in FIG. 68. In some embodiments, an apparatus may include a second diaphragm or other technology to compensate changes of a volume of air inside a hand piece, e.g., due to movement of the ingress shield diaphragm. In some embodiments, a flexible diaphragm may be connected to a needle hub at a needle hub orifice and may be configured to exert a force in a z-direction on a needle hub, e.g., to aid retraction (proximal movement) of a needle hub.

In some embodiments, an apparatus may include a sliding plate, e.g., sliding plate 6901, to (further) protect an interior of a hand piece (e.g., hand piece 120, 220, or 420), e.g., as shown in FIG. 69. A sliding plate may be moveable in an x- and/or y-direction, and may include one or more seals 6903 around a shaft (e.g., a z-axis pushrod 6902), e.g., of a z-actuator connected to a needle hub, e.g., needle hub 6910. A combination of seals (e.g., seals 6903) around sliding plate 6901 may prevent entry of contaminants. In some embodiments, an apparatus may include or may be connected to sealing technologies including one or more bellows (that may be implemented similar to a diaphragm), a face and shaft seal (e.g., analogous to a sliding plate), and/or a sliding sock.

Consumable Detection—Hand Piece

In some embodiments, a needle hub (e.g., needle hub 110, 210, or 410, or needle hub 2710 or 3610) may be implemented as a consumable item, e.g., a needle hub may be discarded after a certain amount of usage. In some embodiments, a needle hub may be replaced after a certain number of insertion/extraction cycles, e.g., about 50 cycles, 100 cycles, 150 cycles, 200 cycles, 250 cycles, 300 cycles, 350 cycles, 400 cycles, 450 cycles, 500 cycles, 600 cycles, 700 cycles, 800 cycles, 900 cycles, or about 1000 cycles. In some embodiments, a needle hub may be replaced after a certain amount of time, e.g., an amount of time a needle hub is disposed in or on an apparatus (e.g., apparatus 100, 200, or 400), e.g., mounted on a needle hub mount. In some embodiments, a needle hub may be a single use item, e.g., a needle hub may not be used again once it has been removed from an apparatus, e.g., disconnected from a needle hub mount. This may improve safety, e.g., by preventing re-use of a needle hub on a different subject, reducing likelihood of infection.

In some embodiments, a needle hub may include a unique identifier. In some embodiments, an identifier may be mounted on or integrated into a needle hub (e.g., a tag, e.g., a chip (e.g., an RFID chip), e.g., in or on a secondary insert 2703 or 3603), and may be used to identify a specific needle hub, e.g., to monitor usage of the needle hub. In some embodiments, a tag may be mounted on a needle hub such that the tag may (directly) contact (e.g., touch) or may otherwise connect to an element, e.g., a read/write element, that is fixed to or is integrated into an apparatus (e.g., a needle hub mount, a hand piece and/or a z-actuator) when the needle hub is mounted, e.g., on a needle hub mount. In some embodiments, a read/write element may be fixed to or movably connected to a hand piece, e.g., mounted on a hand piece shell (e.g., hand piece shell 121, 221, or 421). In some embodiments, a read/write element may be in electronic communication with a digital processing unit that may be operable to receive data from a needle hub tag (e.g., a chip), to process said signals, and/or to write data to the needle hub tag. In some embodiments, a read/write element and needle hub tag may be implemented as a near field communication (NFC) system. In some embodiments, a needle hub tag may include data (e.g., electronic data) stored thereon, e.g., data encoding a unique identifier and/or a certain maximum number of cycles. In some embodiments, when a needle hub is mounted on, e.g., a needle hub mount, a digital processing unit may receive a signal that a needle hub is indeed mounted and may initiate data exchange with the tag via a read/write element. During coring, a digital processing unit may receive data from a z-actuator, e.g., via a sensor mounted thereon or from electric signals to or from a voice coil, and may execute a program to count a number of insertion/extraction cycles. Once a certain number of cycles is reached, e.g., a number pre-programmed into a digital processing unit and/or a number stored on a needle hub tag, a digital processing unit may cause the z-actuator to cease moving, e.g., blocking z-actuation until the needle hub is removed and a new needle hub is mounted. In some embodiments, a digital processing unit may cause a read/write element to write data (e.g., electronic data) to a tag on a needle hub, e.g., data indicating that the needle hub has been mounted and/or used. In some embodiments, a digital processing unit may cause a read/write element to write data to the tag, e.g., data indicating that the needle hub has been mounted and/or used, immediately after a needle hub is mounted on an apparatus. In some embodiments, a digital processing unit of an apparatus may be programmed to prevent z-actuation if such data indicating that the needle hub had been previously mounted is received, e.g., via a read write element. This may prevent the needle hub from being re-used once the needle hub has been dismounted.

In some embodiments, mounting of a needle hub, e.g., mounting a needle hub on a needle hub mount, may be verified, e.g., using a Hall switch device including a Hall effect sensor. A Hall effect sensor is a transducer that may vary its output voltage in response to a magnetic field. In some embodiments, a needle hub may include a magnetic element, e.g., at a proximal end, operable to activate a Hall effect sensor on, e.g., a needle hub mount or hand piece when the needle hub is properly mounted on a hand piece and/or z-actuator. Upon mounting, a Hall effect switch device may receive a signal from the Hall effect sensor and transmit a signal to a digital processing unit, e.g., causing a z-actuator lock to be released.

In some embodiments, a Reed switch device may be used instead of or in addition to a Hall switch device. A Reed switch is an electrical switch activated by the presence of a magnetic field. In some embodiments, a needle hub may include a magnetic element, e.g., at a proximal end, operable to activate a Reed switch on, e.g., a needle hub mount when the needle hub is properly mounted on, e.g., a needle hub mount. A Reed switch device may receive a signal from the Reed switch and transmit a signal to a digital processing unit, e.g., causing a z-actuator lock to be released.

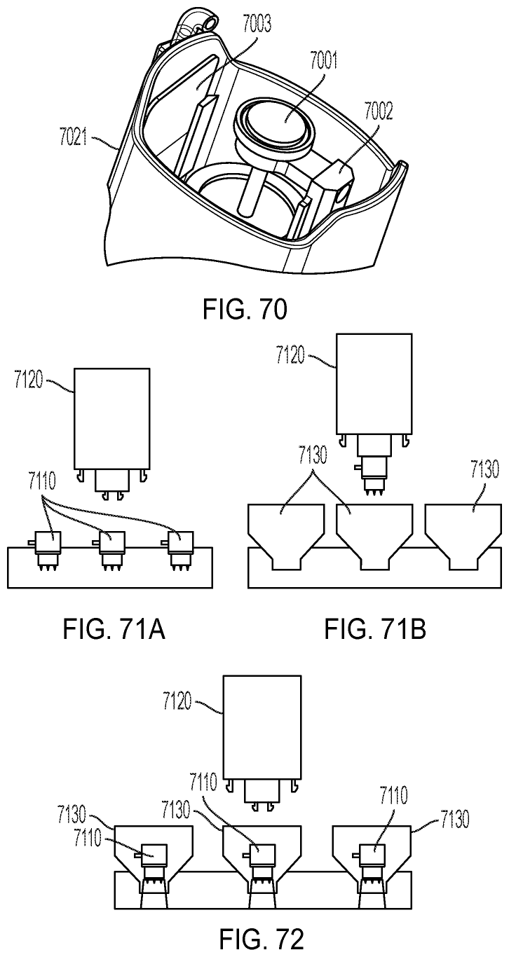

An example embodiment of a needle hub mount that may be used with an apparatus described herein (e.g., apparatus 100, 200, or 400) is shown in FIG. 70. An example needle hub (not shown) is mounted on a needle hub mount, e.g., needle hub mount 7001. In some embodiments, a needle hub mount may be connected to a z-encoder (e.g., encoder 7002), e.g., one or more sensors to monitor z-actuation, e.g., to count z-actuation cycles. A Hall sensor (device that may be used to measure a magnitude of a magnetic field) (not shown) may be mounted in or on a hand piece, e.g., between a hand piece shell (e.g., hand piece shell 7021) and a NFC/Hall effect sensor board e.g., board 7003). A NFC/Hall effect sensor board may include electronic elements for NFC between, e.g., a digital processing unit (e.g., a control unit as described herein), and a needle hub. In some embodiments, a Hall switch device may be used to verify presence and/or proper mounting of a spacer, e.g., a vacuum spacer as described herein (e.g., spacer 4000 or 4100), e.g., upon connection of a spacer to a spacer clip on a hand piece shell.

As described above, in some embodiments, a digital control unit may be programmed and/or used to detect potential damage to a needle hub and to block an apparatus, e.g., actuation of a z-actuator, until the needle hub is replaced. In some embodiments, replacement of a damaged needle hub may be indicated by the removal of a tag (e.g., a chip) associated with a damaged needle hub and connection of a needle hub with a different tag (e.g., chip).

In some embodiments, a needle hub (e.g., needle hub 110, 210, or 410, or needle hub 2710 or 3610) and a spacer, e.g., a vacuum spacer (e.g., spacer 4000 or 4100), may be removable from a hand piece (e.g., hand piece 120, 220, or 420), and may be replaceable together or independently from each other. In some embodiments, a hand piece as described herein (e.g., hand piece 7120) of an apparatus may first be fitted with a needle hub as described herein (e.g., needle hub 7110), e.g., as shown in FIG. 71A, and then be fitted with a spacer as described herein, e.g., a vacuum spacer 7130, e.g., as shown in FIG. 71B. In some embodiments, a hand piece of an apparatus may first be fitted with a spacer, e.g., a vacuum spacer, and then be fitted with a needle hub. In some embodiments, a hand piece (e.g., hand piece 7120) of an apparatus may be fitted with a needle hub (e.g., needle hub 7110) and a spacer, e.g., a vacuum spacer 7130, e.g., in one operation, e.g., as shown in FIG. 72. A needle hub and a spacer, e.g., a vacuum spacer, may be packaged separately or may be packaged together, e.g., for simultaneous attachment of needle hub and spacer. In some embodiments, a packaging device (e.g., a box) may have features, e.g., inserts, that may function as an installation and/or removal tool. In some embodiments, tubing, fluid/debris trap(s) and/or other components of a system or apparatus may be replaceable and/or packaged together with a needle hub and/or spacer.

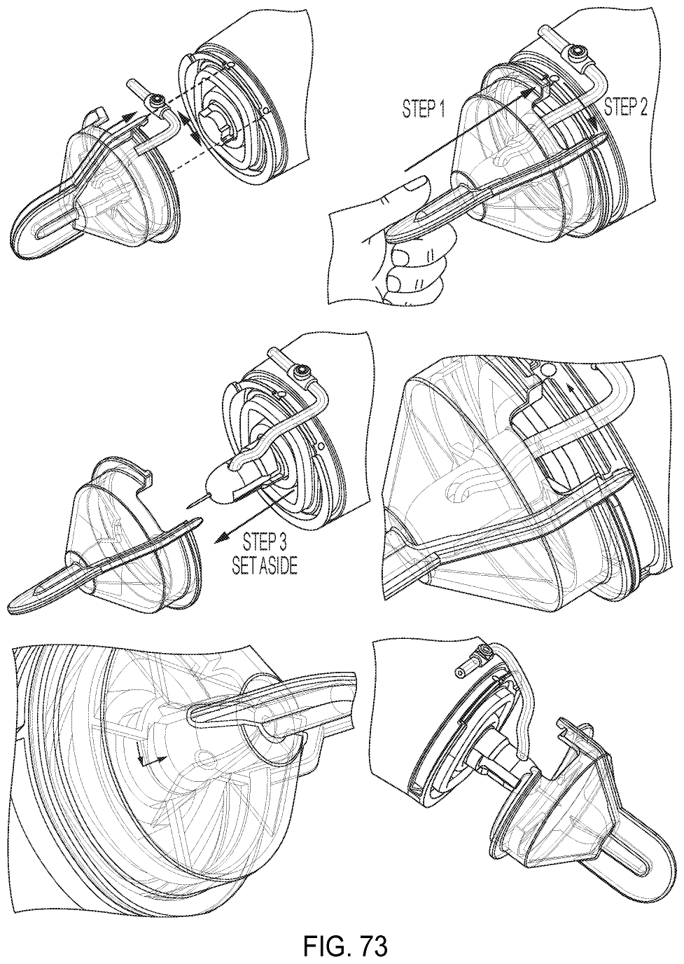

An example needle hub attachment and/or replacement procedure is illustrated in FIG. 73. In some embodiments, a needle hub as described herein may be secured to a hand piece, e.g., a needle hub mount and/or a z-actuator (e.g., voice coil actuator), by way of a magnet (e.g., using a permanent magnet or an electromagnet in a needle hub and/or a needle hub mount). In some embodiments, geometric features on a needle hub and/or a needle hub mount may be used to control orientation of a needle hub. In some embodiments, a needle hub may be mounted by mechanical means such as snaps, threads, and/or bayonet coupling. In some embodiments, packaging implements, e.g., a needle tip protector, maybe used as an installation and/or removal tool. In some embodiments, an installation and/or removal tool may be used to release and/or dismount a needle hub, e.g., by twisting a needle hub a fraction of a turn. In some embodiments, steps of a mounting procedure may be reversed to release and/or dismount a needle hub. In some embodiments, a needle hub may be ejected using an electromagnet, or manual and/or mechanical devices.

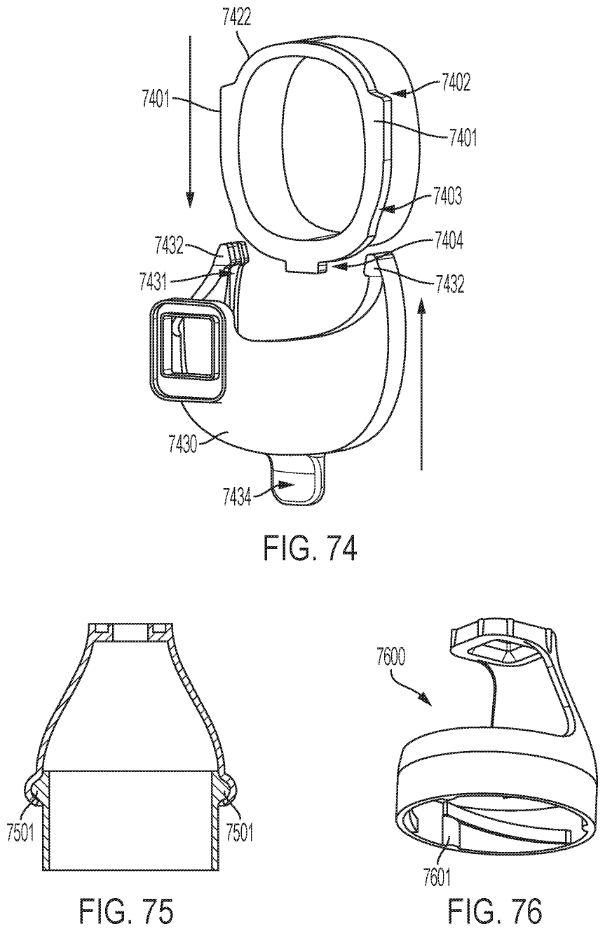

In some embodiments, a spacer, e.g., a vacuum spacer as described herein (e.g., spacer 4000 or 4100), may be removably connected to a hand piece (e.g., hand piece 120, 220, or 420). In some embodiments, a spacer, e.g., vacuum spacer, may include one or more channels at a proximal end that may engage one or more rails at a distal end of a hand piece. In some embodiments, to mount an example spacer on a hand piece, one or more channels 7431 of an example spacer (e.g., spacer 7430) may slide over one or more rails 7401 at a distal end 7422 of a hand piece, e.g., a hand piece shell as described herein, e.g., as shown in FIG. 74. One or more detents 7432 at ends of channels 7431 may engage one or more snaps 7402 to lock spacer 7430 in place. A rail 7401 may have a ramp 7403 for smooth engagement. In some embodiments, a distal end of a hand piece, e.g., distal end 7422 may include a post 7404 engageable with a recess on spacer 7430 (not shown), e.g., to prevent rotation of a spacer, e.g., spacer 7430, e.g., around a z-axis of an actuator. In some embodiments, a spacer, e.g., spacer 7430, may include a pull tab 7434, e.g., to remove spacer 7430 for a hand piece. In some embodiments, a hand piece may include channels at a distal end that may engage one or more rails at a proximal end of a spacer, e.g., a vacuum spacer. In some embodiments, a spacer, e.g., a vacuum spacer may be mounted on a hand piece using a snap on/pinch off mechanism, e.g., mechanism 7501, e.g., as shown in FIG. 75. In some embodiments, a spacer, e.g., a vacuum spacer 7600 may be mounted on a hand piece using a bayonet and/or quarter-turn device with a detent (e.g., detent 7601) to secure a spacer in place, e.g., as shown in FIG. 76. In some embodiments, a spacer, e.g., a vacuum spacer may be mounted on a hand piece be threading a spacer onto a hand piece, by using magnetic connectors on a spacer and/or a hand piece, or by using one or more ball/quick connect joints.

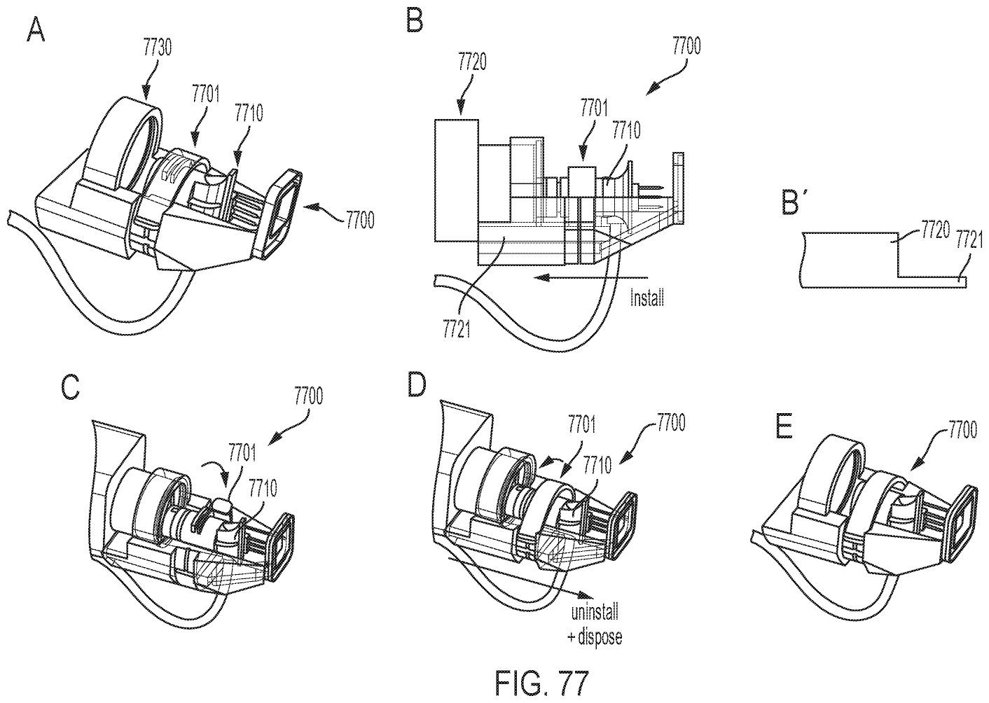

An example spacer mounting system is shown in FIG. 77. In some embodiments, a needle hub and a spacer, e.g., a vacuum spacer, a packaged and/or mounted together. An example joint spacer and needle hub assembly (e.g., assembly 7700) may include a moveable or slideable element, e.g., a circular slideable element (e.g., ring lock 7701) that is moveable from a first position to a second position (FIG. 77A). In some embodiments, when a moveable or slideable element (e.g., ring lock 7701) is in a first position, the moveable or slideable element may engage both a needle hub (e.g., needle hub 7710) and a spacer (e.g., spacer 7730). In some embodiments, when a moveable or slideable element is in a second position, the moveable or slideable element (e.g., ring lock 7701) may engage both a spacer (e.g., spacer 7730) and a hand piece (e.g., hand piece 7720).

In some embodiments, a moveable or slideable element (e.g., ring lock 7701) may be a circular slideable element that may engage one or more grooves or rails on a needle hub, a spacer, and/or a hand piece. When the moveable or slideable element is in a first position, a needle hub is held and/or locked in position relative to a spacer, e.g., the moveable or slideable element engages both a needle hub and a spacer. An example joint spacer and needle hub assembly may be connected to a hand piece (e.g., hand piece 7720), e.g., by sliding a joint spacer and needle hub assembly (e.g., assembly 7700) onto a tang (e.g., tang 7721) at a distal end of a hand piece (e.g., hand piece 7720) (FIG. 77B and FIG. 77B′). During storage and/or sliding of a joint spacer and needle hub assembly (e.g., assembly 7700) onto a tang (e.g., tang 7721), a moveable or slideable element (e.g., ring lock 7701) remains in the first position. When a joint spacer and needle hub assembly (e.g., assembly 7700) is in its final position on a hand piece, e.g., after (fully) sliding the assembly onto the tang (e.g., tang 7721), a moveable or slideable element (e.g., ring lock 7701) may be moved (e.g., rotated) to a second position (FIG. 77C). In some embodiments, one or more grooves or rails on the joint spacer and needle hub assembly may align with one or more grooves or rails on a hand piece, allowing the moveable or slideable element (e.g., ring lock 7701) to engage the grooves or rails on both a spacer (e.g., spacer 7730) and a hand piece (e.g., hand piece 7720), e.g., on tang 7721, thus forming a connection between a spacer and a hand piece. When a moveable or slideable element (e.g., ring lock 7701) is in a second position, a slideable element may disengage grooves or rails a needle hub (e.g., needle hub 7710), and the needle hub may be disconnected from the spacer (e.g., spacer 7730), thus allowing movement (e.g., actuation) of a needle hub (e.g., needle hub 7710). To remove a joint spacer and needle hub assembly (e.g., assembly 7700) from a hand piece, the procedure described above may be reversed (FIG. 77D and FIG. 77E).

Alternative Core Extraction Implements

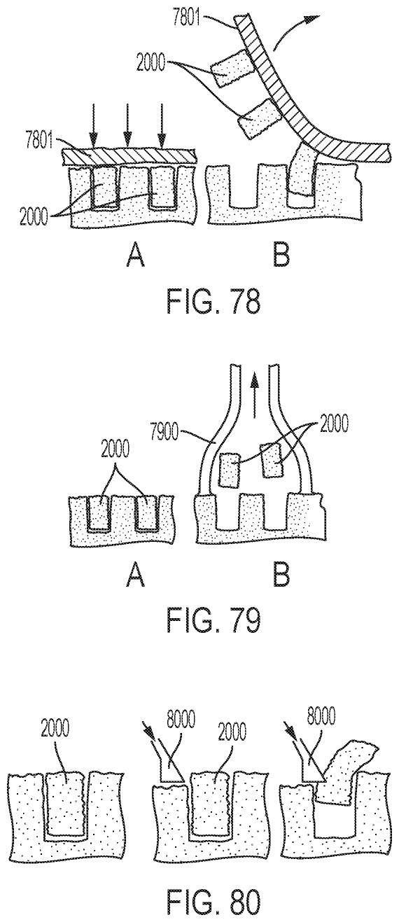

A system as described herein may be implemented for coring without tissue removal. In some embodiments, one or more needles may be used for producing one or more microcores, but needles may be extracted from a tissue with cores remaining in place. In some embodiments, tissue core removal may be accomplished using one or more separate system components. In some embodiments, an adhesive film may be applied to a tissue including mircocores (see FIG. 78A). As an adhesive film (e.g., film 7801) is removed, one or more tissue cores 2000 may remain attached to the film and may be removed (see FIG. 78B). In some embodiments, a separate suction device 7900 may be used to remove one or more tissue cores 2000 after coring, e.g., as shown in FIGS. 79A and B. In some embodiments, a scraping device 8000 may be used to engage one or more microcores as the scraping device is moved across a treated skin surface, thus pulling one or more cores 2000 from their respective holes, e.g., as shown in FIG. 80.

Needles

An example apparatus as described herein includes at least one hollow needle. In some embodiments, an example apparatus as described herein may include at least one hollow needle having at least a first prong. In some embodiments, an angle between a lateral side of a prong and a longitudinal axis of a hollow needle (e.g., a bevel angle α) may be at least about 20 degrees (e.g., the bevel angle α may be greater than about 20 degrees, such as greater than 20 degrees, 22 degrees, 24 degrees, 26 degrees, 28 degrees, 30 degrees, 32 degrees, 34 degrees, 36 degrees, 38 degrees, and 40 degrees, or at an angle of about 20 to about 40 degrees, between 20 to 40 degrees, 20 to 38 degrees, 20 to 36 degrees, 20 to 34 degrees, 20 to 32 degrees, 20 to 30 degrees, 20 to 28 degrees, 20 to 26 degrees, 20 to 24 degrees, 20 to 22 degrees, 22 to 40 degrees, 24 to 40 degrees, 26 to 40 degrees, 28 to 40 degrees, 30 to 40 degrees, 32 to 40 degrees, 34 to 40 degrees, 36 to 40 degrees, or 38 to 40 degrees). In particular, an angle between a lateral side of the prong and a longitudinal axis of the hollow needle (e.g., a bevel angle α) may be about 30 degrees.

In some embodiments, a tip of a prong of a hollow needle may be an edge. In some embodiments, a tip of a prong of a hollow needle is a flat tip having at least two dimensions. In some embodiments, a prong of a hollow needle includes a tip micro-feature. Hollow needles may be constructed to prevent frequent needle damage during use, such as needle tip curling and wear (e.g., becoming dull), needle heel degradation, and needle bending. Hollow needles may be designed to maintain mechanical integrity and durability over a large number of actuation cycles (e.g., actuation cycles greater than 500, 1,000, 2,000, 3,000, 4,000, 5,000, 6,000, 7,000, 8,000, 9,000, 1,0000, 11,000, 12,000, 13,000, 14,000, 15,000, or 20,000). Needles may also effectively remove tissue portions from the skin with high coring rate. In some embodiments, to produce a cosmetic effect in skin tissue, a hollow needle of an apparatus may be inserted into the skin tissue, preferably to a pre-determined depth using a pre-determined force, such that a hollow needle removes a portion of the skin tissue by capturing the portion of the skin tissue in the lumen of the hollow needle.

Prongs

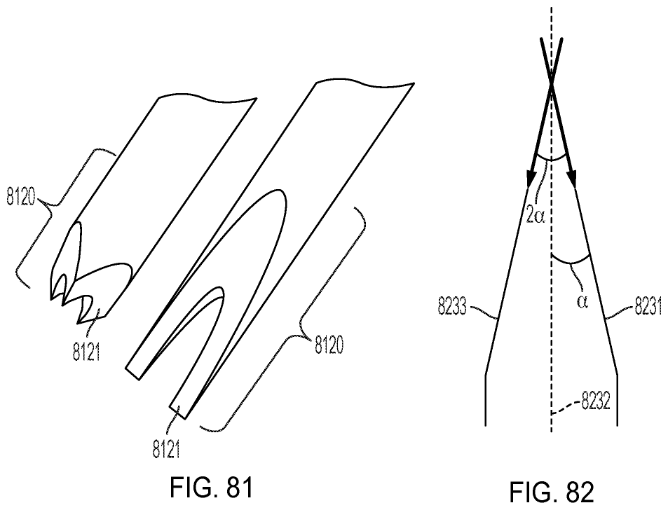

As shown in FIG. 81, distal end 8120 of a hollow needle of an apparatus (e.g., the end of the needle that penetrates the skin tissue) may be shaped to form one or more prongs 8121. In some embodiments, a hollow needle of an apparatus may have one prong at a distal end, two prongs, or more than two prongs (e.g., three, four, five, or six prongs). A hollow needle having one prong may be formed by grinding one side of a distal end of the hollow needle at an angle relative to a longitudinal axis of the hollow needle. A hollow needle having two prongs may be formed by grinding opposite sides of a distal end of the hollow needle at an angle relative to a longitudinal axis of the hollow needle.

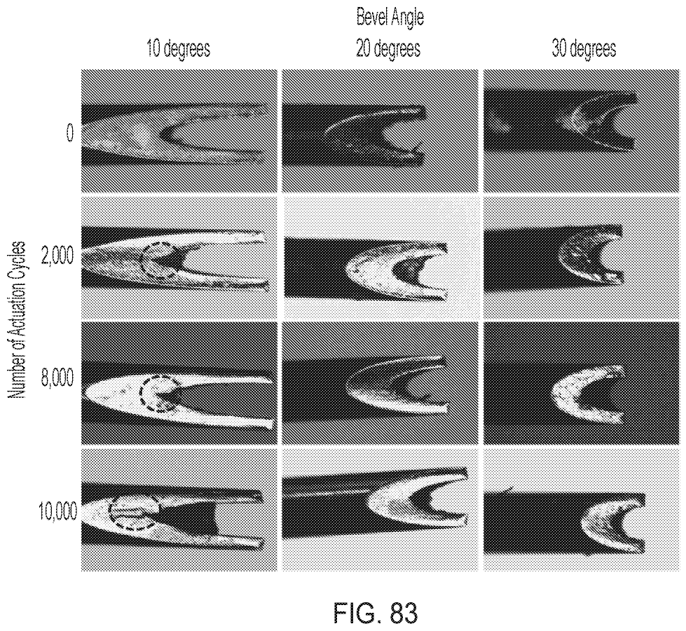

The geometry of a prong at a distal end of a hollow needle may be characterized by a bevel angle. A bevel angle, e.g., angle α as shown in FIG. 82, refers to the angle between lateral side 8231 of the prong and longitudinal axis 8232 of the hollow needle. An angle of “2a” refers to the angle between two lateral sides of the prong of a hollow needle, e.g., the angle between lateral side 8231 and lateral side 8233 of the hollow needle. In some embodiments, a bevel angle α between a lateral side of a prong and a longitudinal axis of the hollow needle may be at least about 20 degrees (e.g., between about 20 and about 40 degrees (e.g., 20, 22, 24, 26, 28, 30, 32, 34, 36, 38, or 40 degrees)). An angle between a lateral side of a prong and a longitudinal axis of a hollow needle may be about 30 degrees. For hollow needles having two or more prongs (e.g., as shown in FIG. 83), each prong may have the same bevel angle or different bevel angles. In some embodiments, for a hollow needle having two prongs, e.g., a first prong and a second prong, an angle between a lateral side of the first prong and a longitudinal axis of the hollow needle may be between about 20 and about 30 degrees (e.g., 20, 22, 24, 26, 28, or 30 degrees) and an angle between a lateral side of the second prong and a longitudinal axis of the hollow needle may be between about 30 and about 40 degrees (e.g., 30, 32, 34, 36, 38, or 30 degrees). For example, a first prong may have a bevel angle α of 20 degrees and a second prong may have a bevel angle α of 30 degrees.

A bevel angle α of at least about 20 degrees or more may improve the mechanical integrity of the needle over several actuation cycles of insertion and withdrawal into skin tissue. Table 1 below shows that a two-prong hollow needle having a 2α bevel angle of 40 degrees (the bevel angle α of each prong is 20 degrees) may reduce the occurrence of needle tip curling relative to a two-prong hollow needle having a 2α bevel angle of 20 degrees (the bevel angle α of each prong is 10 degrees). In an example implementation, a total of five two-prong hollow needles each having a bevel angle α of 10° and five two-prong hollow needles each having a bevel angle α of 20° were tested.

TABLE 1

| Number of | Number of Needles showing Tip Curling | |
|---|---|---|
| Actuation Cycles | 10° Bevel Angle α | 20° Bevel Angle α |
| 5,000 | 1 | 0 |
| 10,000 | 2 | 0 |
| 15,000 | 2 | 0 |
| 20,000 | 3 | 1 |

Additionally, FIG. 83 shows that increasing a needle bevel angle α of a prong may also reduce occurrence of needle heel degradation over a large number of actuation cycles. As show in FIG. 83, a hollow needle having a bevel angle α of 10 degrees displayed signs of needle heel degradation (indicated by dashed circles) before 2,000 actuation cycles, while a hollow needle having a bevel angle α of 20 degrees and a hollow needle having a bevel angle α of 30 degrees showed no apparent sign of needle heel degradation over 10,000 actuation cycles.

A tip of a prong of a hollow needle may be of varying geometries. For example, a tip of a prong may have a sharp point or an edge (e.g., a one-dimensional edge). In some embodiments, for a prong having an edge at the tip, each of the bevel angles of the prong may be at least about 20 degrees (e.g., from about 20 to about 40 degrees (e.g., about 30 degrees)). In some embodiments, for a hollow needle having two or more prongs, e.g., two prongs, the prongs may have different bevel angles (e.g., a bevel angle α of about 20 degrees at the first prong and a bevel angle α of about 30 degrees at the second prong). A tip of a prong may be a flat tip (e.g., a flat tip having two dimensions). For example, a flat tip may have a length and a width. A surface (length/width) of the flat tip of the prong may be at an angle relative to the longitudinal axis of the hollow needle. For example, the surface of the flat tip may be perpendicular to the longitudinal axis of the hollow needle (e.g., at a 90 degree angle relative to the longitudinal axis of the hollow needle) or the surface of the flat tip may be at a non-90 degree angle relative to the longitudinal axis of the hollow needle (e.g., between about 3 to about 89 degrees, such as 3 to 89 degrees, e.g., 3, 6, 9, 12, 15, 18, 21, 24, 27, 30, 33, 36, 39, 42, 45, 48, 51, 54, 57, 60, 63, 66, 69, 72, 75, 78, 81, 84, 87, or 89 degrees). A surface of a flat tip may be level or may have a different geometry, e.g., arc, groove, or non-level. For a prong having a two-dimensional flat tip, each of the bevel angles of the prong may be between about 2 degrees to about 40 degrees (e.g., 2, 4, 6, 8, 10, 12, 14, 16, 18, 20, 22, 24, 26, 28, 30, 32, 34, 36, 38, or 40 degrees). A needle may have one or two prongs each with a two-dimensional flat tip in which one or both of the prongs have a bevel angle α of at least about 20 degrees (e.g., from about 20 to about 40 degrees (e.g., about 30 degrees)). Needles having a one-dimensional edge or a two-dimensional flat tip may exhibit a reduced likelihood of needle tip curling.

Gauges, Inner Diameters, and Lengths

A hollow needle of an apparatus described herein may be of any gauge, including gauges of from 18 to 30 (e.g., 18, 19, 20, 21, 22, 23, 24, 25, 26, 27, 28, 29, or 30 gauge). The gauges of a hollow needle may be from 22 to 25 (e.g., 22, 23, 24, or 25 gauge). A hollow needle of the apparatus may have an inner diameter of from about 0.14 mm to about 0.84 mm (e.g., 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, or 0.84 mm). An inner diameter of a hollow needle may refer to the diameter of the inner lumen of the hollow needle. An inner diameter of a hollow needle may be from about 0.24 mm to about 0.40 mm (e.g., 0.24, 0.25, 0.26, 0.27, 0.28, 0.29, 0.3, 0.31, 0.32, 0.33, 0.34, 0.35, 0.36, 0.37, 0.38, 0.39, or 0.4 mm). An inner diameter of a hollow needle may be from about 0.5 mm to about 2.5 mm (e.g., 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 mm). Accordingly, in some embodiments, a diameter of a portion of skin tissue removed by a hollow needle of an apparatus (e.g., a cored tissue portion) may generally correspond to an inner diameter of a hollow needle.

In some embodiments, an outer and/or inner diameter of a hollow needle may vary across its length, such that the diameter of one region of a hollow needle may be different from the outer and/or inner diameter of another region of the same needle. A change in a diameter across a hollow needle may or may not be continuous. In some embodiments, a hollow needle may or may not be entirely cylindrical. For example, one or more hollow needles may be rectangular, serrated, scalloped, and/or irregular in one or more dimensions and along some or all of their lengths. In some embodiments, the inner lumen diameter may vary along the length of a hollow needle. In some embodiments, a needle may be a swaged hollow needle having a bevel angle α of at least 20 degrees (e.g., between about 20 and about 40 degrees (e.g., 20, 22, 24, 26, 28, 30, 32, 34, 36, 38, or 40 degrees)) and a variable inner lumen diameter over its length. A swaged hollow needle may have a smaller diameter near the distal end of the hollow needle (e.g., near the end of the needle that penetrates the skin tissue). In some embodiments, an inner diameter may be wider at the proximal end of a hollow needle (e.g., away from the tip that penetrates the skin). This may facilitate the removal of a cored tissue portion from the hollow needle, may limit the need for clearing of the hollow needle, and/or may reduce the occurrence of needle clogging.

A hollow needle of an apparatus may be of varying lengths and may have varying active lengths (e.g., the length of a hollow needle configured to penetrate the skin tissue). Active lengths may vary from about 0.5 mm to about 10 mm (e.g., 0.5, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, or 10 mm) and may be adjustable/selectable with manual or automatic controls (e.g., as described herein, e.g., using a scroll wheel or an actuation mechanism such as an electromagnetic actuator). Active lengths of a hollow needle may be adjusted and selected depending on a skin area needing treatment. In some embodiments, a hollow needle with an active length from about 0.5 mm to about 2 mm (e.g., 0.5, 0.6, 0.8, 1, 1.2, 1.4, 1.6, 1.8, or 2 mm) may be used to treat thin skin, e.g., skin of an eyelid. The thickness of the epidermal and dermal layers of the skin of an eyelid may be from about 0.5 mm to about 1 mm (e.g., 0.5, 0.6, 0.8, or 1). Hollow needles with active lengths from about 5 mm to about 10 mm (e.g., 5, 6, 7, 8, 9, or 10 mm) may be used to treat thick skin, e.g., skin of the back or scar tissue, which may be thicker than healthy skin tissue. The thickness of an epidermal layer of skin may be from about 0.05 to about 2 mm (e.g., 0.05 to 2, 0.05 to 1.95, 0.05 to 1.9, 0.05 to 1.85, 0.05 to 1.8, 0.05 to 1.75, 0.05 to 1.7, 0.05 to 1.65, 0.05 to 1.6, 0.05 to 1.55, 0.05 to 1.5, 0.05 to 1.45, 0.05 to 1.4, 0.05 to 1.35, 0.05 to 1.3, 0.05 to 1.25, 0.05 to 1.2, 0.05 to 1.15, 0.05 to 1.1, 0.05 to 1.05, 0.05 to 1, 0.05 to 0.95, 0.05 to 0.9, 0.05 to 0.85, 0.05 to 0.8, 0.05 to 0.75, 0.05 to 0.7, 0.05 to 0.65, 0.05 to 0.6, 0.05 to 0.55, 0.05 to 0.5, 0.05 to 0.45, 0.05 to 0.4, 0.05 to 0.35, 0.05 to 0.3, 0.05 to 0.25, 0.05 to 0.2, 0.05 to 0.15, 0.05 to 0.1, 0.1 to 2, 0.15 to 2, 0.2 to 2, 0.25 to 2, 0.3 to 2, 0.35 to 2, 0.4 to 2, 0.45 to 2, 0.5 to 2, 0.55 to 2, 0.6 to 2, 0.65 to 2, 0.7 to 2, 0.75 to 2, 0.8 to 2, 0.85 to 2, 0.9 to 2, 0.95 to 2, 1 to 2, 1.05 to 2, 1.15 to 2, 1.2 to 2, 1.25 to 2, 1.3 to 2, 1.35 to 2, 1.4 to 2, 1.45 to 2, 1.5 to 2, 1.55 to 2, 1.6 to 2, 1.65 to 2, 1.7 to 2, 1.75 to 2, 1.8 to 2, 1.85 to 2, 1.9 to 2, or 1.95 to 2 mm). The thickness of a dermal layer of skin may be from 2 to 8 mm (e.g., 2 to 8, 2 to 7.5, 2 to 7, 2 to 6.5, 2 to 6, 2 to 5.5, 2 to 5, 2 to 4.5, 2 to 4, 2 to 3.5, 2 to 3, 2 to 2.5, 2.5 to 8, 3 to 8, 3.5 to 8, 4 to 8, 4.5 to 8, 5 to 8, 5.5 to 8, 6 to 8, 6.5 to 8, 7 to 8, or 7.5 to 8 mm). Active lengths of a hollow needle may be adjusted and selected to penetrate the epidermal and/or the dermal layer of skin.

In some embodiments, active lengths of a hollow needle may also be adjusted using one or more spacers, which are described in detail further herein. Hollow needle parameters may be selected based on the area of skin and the condition to be treated. For example, treatment of thin, lax skin on the cheeks may benefit from a hollow needle having an active length of about 2 mm and medium gauge (e.g., 25 gauge), while treatment of thick skin on the back or treatment of scar tissue may benefit from a hollow needle having an active length closer to 5 mm and a thicker gauge (e.g., 22 gauge). A hollow needle of an apparatus may be configured to extend to varying depths of the skin tissue. In some embodiments, depth of penetration of a hollow needle may be determined by the active length (e.g., from about 2 mm to about 5 mm) of a hollow needle. In some embodiments, a hollow needle may be configured to extend (i) into the dermal layer, (ii) through the entire dermal layer to the junction of the dermal layer and the subcutaneous fat layer, and/or (iii) into the subcutaneous fat layer.

Needle Coating

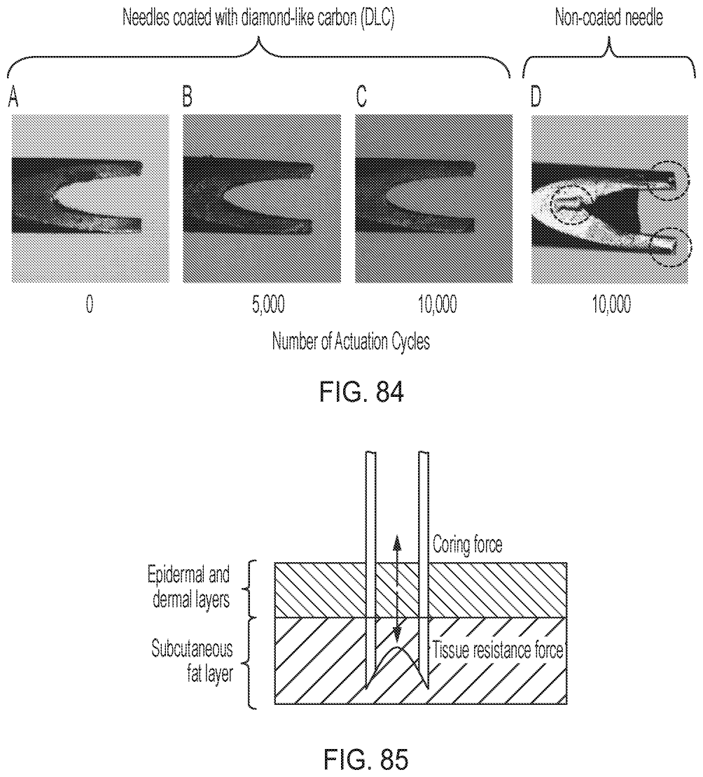

In some embodiments, a hollow needle may be coated with a material (e.g., a hard material) that may improve or maintain the mechanical integrity, durability, reliability, and/or affect mechanical, biological, or electrical properties of the hollow needle. A coating material may help to prevent damage, abrasion, and wear and tear of the needle tip and heel during repeated insertions into and withdrawals from skin tissue. Examples of materials (e.g., a hard material) that may be used to coat a hollow needle of the apparatus include, but are not limited to, TiN, TiCN, TiAlN, ZrN, and diamond-like carbon (DLC). A hard material may be applied as a coating to the outside surface of a hollow needle, the inner surface (e.g., the surface of the inner lumen) of a hollow needle, or both surfaces. Results from an experiment shown in FIGS. 84A-84C show that a hollow needle coated with DLC exhibited a reduction in needle heel and tip degradation over 10,000 actuation cycles of insertions and withdrawals into pig skin, while a non-coated hollow needle showed needle heel and tip degradation (indicated by dashed circles) over 10,000 actuation cycles of insertions and withdrawals into pig skin (FIG. 84D).

Surface of Needle Lumen

A lumen surface of a hollow needle may affect coring force, coring rate, and/or insertion force of the hollow needle. Without wishing to be bound by theory, the friction between a lumen surface and a cored tissue portion may determine the coring force, coring rate, and insertion force. Hollow needles described herein may be designed to maximize coring rate and minimize hollow needle insertions that do not result in cored tissue removal. A tissue portion detaches from skin when a coring force (e.g., the force applied by the hollow needle of the apparatus to the cored tissue portion as the needle is being withdrawn from the skin) exceeds a tissue resistance force, which may be determined by the connection of the tissue portion to its surrounding tissue. For example, when a hollow needle is fully inserted through the dermal layer of the skin, a tissue resistance force may be determined by the connection between the tissue portion in the lumen of the needle and the subcutaneous fat layer. Accordingly, when coring force exceeds tissue resistance force, the cored tissue portion may be captured in the lumen of the hollow needle and removed from the skin (see FIG. 85). A rough lumen surface may increase friction between a cored tissue portion and a lumen surface, which may result in increased insertion force, increased coring force, and/or increased coring rate. Lubrication of a lumen surface may reduce friction between a cored tissue portion and a lumen surface, which may result in decreased insertion force, decreased coring force, and decreased coring rate. An overly rough and uneven lumen surface may lead to higher occurrence of needle degradation (e.g., needle heel and/or tip degradations), may cause difficulty in removing cored tissue portions from a lumen, and/or may cause needle clogging, compared to a needle having smooth and/or even lumen surface. The degree of roughness of a lumen surface may be optimized to increase coring force and/or coring rate without compromising the durability of the needle, the insertion force, the ability to remove tissue from the needle lumen, and the resistance of a needle to degradation (e.g., needle heel and tip degradation).

In some embodiments, hollow needles and methods may have a coring rate of at least about 5% (e.g., from about 5% to about 100%, such as 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 95%, 15% to 95%, 20% to 95%, 25% to 95%, 30% to 95%, 35% to 95%, 40% to 95%, 45% to 95%, 50% to 95%, 55% to 95%, 60% to 95%, 65% to 95%, 70% to 95%, 75% to 95%, 80% to 95%, 85% to 95%, or 90% to 95%).

In some embodiments, hollow needles and methods may exert a coring force of about 3 N to about 10 N (e.g., 3, 3.5, 4, 4.5, 5, 5.5, 6, 6.5, 7, 7.5, 8, 8.5, 9, 9.5, or 10 N). In some embodiments, a two-prong hollow needle having a bevel angle α of 20 degrees may exert a coring force of about 3 N to about 10 N (e.g., 3, 3.5, 4, 4.5, 5, 5.5, 6, 6.5, 7, 7.5, 8, 8.5, 9, 9.5, or 10 N).

A coating material and/or a lubricant may affect the degree of roughness of the lumen surface, and thus friction between the lumen surface and a cored tissue portion. A lumen surface of a hollow needle may be polished by running a lubricant or polishing media though the hollow needle to reduce the roughness of the lumen surface. Examples of lubricants include, but are not limited to, salt-based lubricants (e.g., buffered saline solutions (e.g., PBS)), sugar-based lubricants (e.g., sucrose and glucose solutions), and/or surfactant-based lubricants (e.g., solutions containing Tween20). The degree of roughness of the lumen surface of the hollow needle may also be affected by the manufacturing process used to make the hollow needle. Table 2 below shows lumen surface roughness measured in Ra (arithmetic average of roughness profile) and Rz (mean roughness depth) of hollow needles made using single plug, double plug, and/or sunk manufacturing processes. The lumen surface of hollow needles made using double plug process may be smoother (lower Ra and Rz values) than the lumen surface of hollow needles made using single plug process.

TABLE 2

| Manufacturing Process | Ra | Rz |
|---|---|---|
| Single plug | 53 | 299 |
| Double plug | 37 | 206 |
| Sunk | 56 | 330 |

Array Patterns

One or more hollow needles of a system and/or apparatus as described herein may be arranged, e.g., on a needle hub, to form an array pattern in skin upon removal of portions of skin tissue. In some embodiments, an array pattern may include holes in one or more rows or in a random or semi-random spatial distribution. Size and geometry of an array pattern may be generated based on an area of skin and condition being treated. In some embodiments, a small array pattern may be generated for treatment of the peri-oral area, while a large array pattern may be suitable for treatment of the abdomen. In some embodiments, an array pattern may be generated using different numbers and/or arrangements of a plurality of hollow needles. In some embodiments, an array pattern may be generated using one hollow needle, which may undergo multiple actuation cycles and be translated across a surface of a skin region, e.g., by an x-actuator and/or y-actuator to generate an array pattern. In some embodiments, an array pattern may be generated using a plurality of hollow needles (e.g., an array of hollow needles), which may undergo one or more actuation cycles to generate an array pattern. A number of actuation cycles needed to generate an array pattern of holes in skin tissue may be determined by the size of the array pattern, the gauge and/or inner and/or outer diameter of a hollow needle, the number of hollow needles, size distribution of a plurality of needles of different sizes, and/or an amount of skin tissue to be removed, e.g., an areal fraction of skin tissue removed. An “areal fraction” of tissue removed refers to the fraction of skin tissue surface covered by holes generated by one or more hollow needle(s) of an apparatus. In other words, an areal fraction of tissue removed refers to the ratio of the area covered by the total amount of cored tissue portions to the total skin treatment area. In some embodiments, one or more hollow needles may be used or configured to remove an areal fraction of about 0.01 to about 0.65 (e.g., 0.01, 0.02, 0.03, 0.04, 0.05, 0.10, 0.15, 0.20, 0.25, 0.30, 0.35, 0.40, 0.45, 0.50, 0.55, 0.60, or 0.65) of tissue within a treatment area. In some embodiments, one or more hollow needles may be used or configured to remove an areal fraction of less than about 0.1, such as about 0.01 to about 0.05 (e.g., 0.01, 0.015, 0.02, 0.025, 0.03, 0.035, 0.04, 0.045, or 0.05) of tissue within a treatment area. In some embodiments, one or more hollow needles may be used or configured to remove an areal fraction of about 0.02 to about 0.03 (e.g., 0.02, 0.021, 0.022, 0.023, 0.024, 0.025, 0.026, 0.027, 0.028, 0.029, or 0.03, e.g., 0.025) of tissue within a treatment area. In some embodiments, an areal fraction of about 0.01 to about 0.65 (e.g., 0.01, 0.02, 0.03, 0.04, 0.05, 0.10, 0.15, 0.20, 0.25, 0.30, 0.35, 0.40, 0.45, 0.50, 0.55, 0.60, or 0.65) of tissue may be removed within a treatment area, e.g., for wrinkle reduction. In some embodiments, an areal fraction of about 0.02 to about 0.03 (e.g., 0.02, 0.021, 0.022, 0.023, 0.024, 0.025, 0.026, 0.027, 0.028, 0.029, or 0.03, e.g., 0.025) of tissue may be removed within a treatment area, e.g., for wrinkle reduction. Table 3 below shows an example number of actuation cycles required for the treatment of different body areas using a 24 gauge hollow needle.

TABLE 3

| | Total | Areal | Number |
|---|---|---|---|
| | Treatment | Fraction | of |
| Treatment | Area | of Tissue | Actuation |
| Site | (cm²) | Removed | Cycles |
| Cheek | 120 | 0.1 | 15,782 |
| Upper lip | 10 | 0.1 | 1,315 |
| Knee | 120 | 0.1 | 15,782 |
| Hand | 100 | 0.1 | 13,151 |

An apparatus as described herein may be configured for detachable attachment to one or more hollow needles having the same or different configurations. In some embodiments, an apparatus may have as few as 1 or as many as hundreds of hollow needles. In some embodiments, 1-100 hollow needles may be present (e.g., 1-10, 1-20, 1-30, 1-40, 1-50, 1-60, 1-70, 1-80, 1-90, 1-100, 3-10, 3-20, 3-30, 3-40, 3-50, 3-60, 3-70, 3-80, 3-90, 3-100, 5-10, 5-20, 5-30, 5-40, 5-50, 5-60, 5-70, 5-80, 5-90, 5-100, 10-20, 10-40, 10-60, 10-80, 10-100, 20-40, 20-60, 20-80, 20-100, 40-60, 40-80, 40-100, 60-80, 60-100, or 80-100 hollow needles). The use of an array of a plurality of hollow needles to generate an array pattern may facilitate skin treatment over larger areas and/or in less time.

In some embodiments, a minimum distance between two hollow needles in an array of hollow needles may be between about 0.1 mm to about 50 mm (e.g., from 0.1 mm to 0.2 mm, 0.1 mm to 0.5 mm, 0.1 mm to 1 mm, 0.1 mm to 2 mm, 0.1 mm to 5 mm, 0.1 mm to 10 mm, 0.1 mm to 15 mm, 0.1 mm to 20 mm, 0.1 mm to 30 mm, 0.1 mm to 40 mm, 0.1 mm to 50 mm, 0.2 mm to 0.5 mm, 0.2 mm to 1 mm, 0.2 mm to 2 mm, 0.2 mm to 5 mm, 0.2 mm to 10 mm, 0.2 mm to 15 mm, 0.2 mm to 20 mm, 0.2 mm to 30 mm, 0.2 mm to 40 mm, 0.2 mm to 50 mm, 0.5 mm to 1 mm, 0.5 mm to 2 mm, 0.5 mm to 5 mm, 0.5 mm to 10 mm, 0.5 mm to 15 mm, 0.5 mm to 20 mm, 0.5 mm to 30 mm, 0.5 mm to 40 mm, 0.5 mm to 50 mm, 1 mm to 2 mm, 1 mm to 5 mm, 1 mm to 10 mm, 1 mm to 15 mm, 1 mm to 20 mm, 1 mm to 30 mm, 1 mm to 40 mm, 1 mm to 50 mm, 2 mm to 5 mm, 2 mm to 10 mm, 2 mm to 15 mm, 2 mm to 20 mm, 2 mm to 30 mm, 2 mm to 40 mm, 2 mm to 50 mm, 5 mm to 10 mm, 5 mm to 15 mm, 5 mm to 20 mm, 5 mm to 30 mm, 5 mm to 40 mm, 5 mm to 50 mm, 10 mm to 15 mm, 10 mm to 20 mm, 10 mm to 30 mm, 10 mm to 40 mm, 10 mm to 50 mm, 15 mm to 20 mm, 15 mm to 30 mm, 15 mm to 40 mm, 15 mm to 50 mm, 20 mm to 30 mm, 20 mm to 40 mm, 20 mm to 50 mm, 30 mm to 40 mm, 30 mm to 50 mm, or 40 mm to 50 mm). In some embodiments, a distance between two hollow needles in an array of hollow needles is less than about 15 mm. In some embodiments, a minimum distance may correspond to the minimal size of an array pattern, while the maximum distance may correspond to the maximum size or dimension of an array pattern.

Coring procedures may be adapted and/or optimized, e.g., to adapt coring to specific tissue types, e.g., wrinkles, scars, or dog ears, or to trace certain features, e.g., scars or tumors. Coring depth, hole density, and/or patterns may be adapted and/or optimized. In some embodiments, array patterns of different sizes and geometries may be generated based on the area of treatment and the skin condition being treated. In some embodiments, array patterns may also be generated for compatibility with actuation mechanisms and/or control electronics of a given apparatus. In some embodiments, actuation mechanisms and/or control electronics of an apparatus may be selected for compatibility with a desired array pattern size and/or geometry. In some embodiments, a long, linear array pattern may be generated using a translating mechanism with driving wheels, while a large, rectangular array may be generated using an x- and/or y-actuator to drive the hollow needle(s) across skin. In some embodiments, a pattern may be pre-programmed or adapted during a procedure, e.g., during a coring process, e.g., to adapt and/or optimize treatment in real time. In some embodiments, adaptation and/or optimization of a coring procedure may be based on tissue characteristics. In some embodiments, adaptation and/or optimization may be carried out based on voice coil data (e.g., kinematics and/or electronics), or may be carried out based on other data, e.g. acoustic, optical, or radiofrequency data obtained before, during, and/or after a coring procedure.

In an example apparatus, one or more hollow needles may be configured to provide from about 10 to about 10000 cored tissue portions or more per cm² area (e.g., 10 to 50, 10 to 100, 10 to 200, 10 to 300, 10 to 400, 10 to 500, 10 to 600, 10 to 700, 10 to 800, 10 to 900, 10 to 1000, 10 to 2000, 10 to 4000, 10 to 6000, 10 to 8000, 10 to 10000, 50 to 100, 50 to 200, 50 to 300, 50 to 400, 50 to 500, 50 to 600, 50 to 700, 50 to 800, 50 to 900, 50 to 1000, 50 to 2000, 50 to 4000, 510 to 6000, 50 to 8000, 50 to 10000, 100 to 200, 100 to 300, 100 to 400, 100 to 500, 100 to 600, 100 to 700, 100 to 800, 100 to 900, 100 to 1000, 100 to 2000, 100 to 4000, 100 to 6000, 100 to 8000, 100 to 10000, 200 to 300, 200 to 400, 200 to 500, 200 to 600, 200 to 700, 200 to 800, 200 to 900, 200 to 1000, 200 to 2000, 200 to 4000, 200 to 6000, 200 to 8000, 200 to 10000, 300 to 400, 300 to 500, 300 to 600, 300 to 700, 300 to 800, 300 to 900, 300 to 1000, 300 to 2000, 300 to 4000, 300 to 6000, 300 to 8000, 300 to 10000, 400 to 500, 400 to 600, 400 to 700, 400 to 800, 400 to 900, 400 to 1000, 400 to 2000, 400 to 4000, 400 to 6000, 400 to 8000, 400 to 10000, 500 to 600, 500 to 700, 500 to 800, 500 to 900, 500 to 1000, 500 to 2000, 500 to 4000, 500 to 6000, 500 to 8000, 500 to 10000, 600 to 700, 600 to 800, 600 to 900, 600 to 1000, 600 to 2000, 600 to 4000, 600 to 6000, 600 to 8000, 600 to 10000, 700 to 800, 700 to 900, 700 to 1000, 700 to 2000, 700 to 4000, 700 to 6000, 700 to 8000, 700 to 10000, 800 to 900, 800 to 1000, 800 to 2000, 800 to 4000, 800 to 6000, 800 to 8000, 800 to 10000, 900 to 1000, 900 to 2000, 900 to 4000, 900 to 6000, 900 to 8000, 900 to 10000, 1000 to 2000, 1000 to 4000, 1000 to 6000, 1000 to 8000, 1000 to 10000, 2000 to 4000, 2000 to 6000, 2000 to 8000, 2000 to 10000, 4000 to 6000, 4000 to 8000, 4000 to 10000, 6000 to 8000, 6000 to 10000, or 8000 to 10000 tissue portions per cm² area) of the skin region to which the apparatus is applied (e.g., the treatment area).

Base Unit and User Interface

An apparatus as described herein (e.g., apparatus 100, 200, or 400) may be in communication with a base unit and/or control unit, which may include, e.g., a user interface, a power supply, control electronics, e.g., a digital processing unit, mechanisms to drive operation of the apparatus, and other components. A base unit may include a computer including, e.g., a digital processing unit, which may be programmed to operate and/or control any or all aspects of a system or an apparatus (e.g., apparatus 100, 200, or 400) as described herein. A base unit may include one or more pumps, valves, traps, actuators, switches, and/or tubing, e.g., to generate low pressure or (partial) vacuum in a system and/or to move fluids through one or more components of a system and/or apparatus.

A user interface in a base unit may include buttons, keys, switches, toggles, spin-wheels, screens, touch screens, keyboards, cursors, dials, indicators, displays, and/or other components, and may be connected to one or more digital processing units. In some embodiments, a user interface may be configured or programmed to indicate proper couplings and/or attachments of one or more components of a system, e.g., proper couplings and/or attachments of a support base, a z-actuator (e.g., a voice coil), one or more hollow needles, a fluid conduit, an aspiration tube, a trap, a low pressure and/or (partial) vacuum generation system, a pressure generating source (e.g., a vacuum pump), and or a needle assembly. In some embodiments, a user interface may be configured or programmed to indicate, e.g., charged and/or powered status of an apparatus, mode and/or position of hollow needle(s), application of high (e.g., positive) pressure or low pressure (e.g., partial vacuum), actuation of one or more apparatus components, and/or other indicia. In some embodiments, a user interface may be configured or programmed to provide information about the number and kind of hollow needle(s) of an apparatus, a treatment area, treatment coverage (e.g., areal fraction of skin surface area removed), arrangement of one or more hollow needles, potential depth of penetration by hollow needle(s), mechanism or mode of operation, use count of the hollow needle(s), and other information. In some embodiments, a user interface may include implements for adjustment of parameters and/or operation mode, application of high (e.g., positive) pressure or low pressure (e.g., partial vacuum), and/or activation of penetration into the skin by one or more hollow needle(s). In some embodiments, a user interface may also be configured or programmed to transmit and/or receive information from another unit. For example, user actions at a user interface on an apparatus may be reflected by a user interface of the base unit, or vice versa.

A base unit may include buttons, keys, switches (e.g., hand switches or foot switches), toggles, spin-wheels, and/or other activation mechanisms for adjustment of parameters and/or operation mode, adjust pressure, e.g., application of high (e.g., positive) pressure or low pressure (e. g., partial vacuum), depth and/or duration of penetration into skin by one or more hollow needle(s), and/or powering on or off of a base unit and/or pressure generating source. In some embodiments, these components may be integrated into a user interface of the base unit. In some embodiments, a base unit may include one or more foot switches that may allow a user to operate one or more functions of a system, e.g., low pressure system and/or z-actuation without use of a user's hands, e.g., while maintaining grip on a hand piece. In some embodiments, one or more feedback devices and/or controls may be integrated into an apparatus, e.g., a hand piece, and may include lights, screens, vibrating implements and/or audio signal generators.

In some embodiments, the base unit may include electronics to control operation of the apparatus, pressure generating source, and/or other components couple to the apparatus. For example, the base unit may include one or more microcontrollers, programmable logic, discrete elements, and/or other components. The base unit may have one or more power supplies, or may include one or more connections to power supply external to the base unit. Power supplies may include batteries, alternators, generators, and/or other components. In some embodiments, a base unit may include one or more devices for conversion of main power (alternating current) to direct current for system operation. In some embodiments, a base unit may include a battery charging station for use with a battery-powered apparatus.

In some embodiments, a base unit may include a user interface that may indicate, e.g., that a hollow needle is properly installed in a needle hub, that a needle hub is properly coupled to an actuation unit, that an apparatus is charged or otherwise powered (e.g., the amount of battery life remaining), that one or more hollow needles are in an extended or retracted position, that a pressure generating source is coupled to an apparatus, that a fill level of a trap for collecting cored tissue portions, and/or other information. In some embodiments, a user interface may include information about an apparatus, such as the number of hollow needle(s) of the apparatus, an arrangement of the hollow needle(s), a potential depth of tissue penetration by the hollow needle(s), a mechanism or mode of operation, and/or other information. In some embodiments, a user interface may include buttons, keys, switches, toggles, spin-wheels, LED displays, and/or touch screens that allow a user to observe and change various parameters or configurations during operation of the apparatus, to activate and/or de-activate a pressure generating source, and/or to initiate penetration into the skin by one or more hollow needle(s). In some embodiments, a user interface may also be configured to transmit and/or receive information from another unit, such as a computer, e.g., a digital processing unit.

In some embodiments, a base unit is or comprises a cart, e.g., including a structure moveable, e.g., on wheels. In some embodiments, one or more pumps, traps, user interfaces are mounted on a cart. In some embodiments, an apparatus is connected to a base unit, e.g., a cart, via a moveable articulated arm, e.g., to support an apparatus or hand piece and/or facilitate movement and/or stabilization of an apparatus or hand piece.

Materials

The technologies described herein (e.g., hollow needles, needle hubs, actuation units, apparatuses, kits, and methods described herein) may include (e.g., be comprises of/made from) any material. For example, a needle hub may include and/or be formed from any polymer or plastic. Such materials may include alginate, benzyl hyaluronate, carboxymethylcellulose, cellulose acetate, chitosan, collagen, dextran, epoxy, gelatin, hyaluronic acid, hydrocolloids, nylon (e.g., nylon 6 or PA6), pectin, poly (3-hydroxyl butyrate-co-poly (3-hydroxyl valerate), polyalkanes, polyalkene, polyalkynes, polyacrylate (PA), polyacrylonitrile (PAN), polybenzimidazole (PBI), polycarbonate (PC), polycaprolactone (PCL), polyester (PE), polyethylene glycol (PEG), polyethylene oxide (PEO), PEO/polycarbonate/polyurethane (PEO/PC/PU), poly(ethylene-co-vinyl acetate) (PEVA), PEVA/polylactic acid (PEVA/PLA), polyethylene, polypropylene, poly (ethylene terephthalate) (PET), PET/poly (ethylene naphthalate) (PET/PEN) polyglactin, polyglycolic acid (PGA), polyglycolic acid/polylactic acid (PGA/PLA), polyimide (PI), polylactic acid (PLA), poly-L-lactide (PLLA), PLLA/PC/polyvinylcarbazole (PLLA/PC/PVCB), poly (β-malic acid)-copolymers (PMLA), polymethacrylate (PMA), poly (methyl methacrylate) (PMMA), polystyrene (PS), polyurethane (PU), poly (vinyl alcohol) (PVA), polyvinylcarbazole (PVCB), polyvinyl chloride (PVC), polyvinylidenedifluoride (PVDF), polyvinylpyrrolidone (PVP), silicone, rayon, polytetrafluoroethylene (PTFE), polyether ether ketone (PEEK), or combinations thereof. Polymers and/or plastics that may be used in the apparatus or system as described herein may be composite materials in which additives to the polymers and/or plastics, such as ceramics or particles, alter the mechanical properties.

Elements of the technologies described herein (e.g., all or a portion of the apparatus, such as all or a portion of the needle assembly, the actuation unit, or other components) may also include and/or be formed from any useful metal or metal alloy. For example, in some embodiments, a hollow needle may be a metallic needle. Metals and alloys that may be used in the apparatus or system as described herein include stainless steel; titanium; a nickel-titanium (NiTi) alloy; a nickel-titanium-niobium (NiTiNb) alloy; a nickel-iron-gallium (NiFeGa) alloy; a nickel-manganese-gallium (NiMnGa) alloy; a copper-aluminum-nickel (CuAlNi) allow; a copper-zinc (CuZn) alloy; a copper-tin (CuSn) alloy; a copper-zinc-aluminum (CuZnAl) alloy; a copper-zinc-silicon (CuZnSi) alloy; a copper-zinc-tin (CuZnSn) alloy; a copper-manganese alloy; a gold-cadmium (AuCd) alloy; a silver-cadmium (AgCd) alloy; an iron-platinum (FePt) alloy; an iron-manganese-silicon (FeMnSi) alloy; a cobalt-nickel-aluminum (CoNiAl) alloy; a cobalt-nickel-gallium (CoNiGa) alloy; or a titanium-palladium (TiPd) alloy. Elements of the technologies described herein may include and/or be formed from glass. For example, an apparatus may include one or more glass hollow needles.

The systems, hollow needles, needle assemblies, actuation units, apparatuses, kits, and/or methods described herein may include one or more adhesives. An adhesive may be located on a surface, between elements, or otherwise adhered to an element, e.g., of an apparatus as described herein. Example adhesives include a biocompatible matrix (e.g., those including at least one of collagen (e.g., a collagen sponge), low melting agarose (LMA), polylactic acid (PLA), and/or hyaluronic acid (e.g., hyaluranon); a photosensitizer (e.g., Rose Bengal, riboflavin-5-phosphate (R-5-P), methylene blue (MB), N-hydroxypyridine-2-(1H)-thione (N-HTP), a porphyrin, or a chlorin, as well as precursors thereof); a photochemical agent (e.g., 1,8 naphthalimide); a synthetic glue (e.g., a cyanoacrylate adhesive, a polyethylene glycol adhesive, or a gelatin-resorcinol-formaldehyde adhesive); a biologic sealant (e.g., a mixture of riboflavin-5-phosphate and fibrinogen, a fibrin-based sealant, an albumin-based sealant, or a starch-based sealant); or a hook or loop and eye system (e.g., as used for Velcro®). In some embodiments, an adhesive is biodegradable.

In some embodiments, an adhesive may be a pressure-sensitive adhesive (PSA). The properties of pressure sensitive adhesives are governed by three parameters: tack (initial adhesion), peel strength (adhesion), and shear strength (cohesion). Pressure-sensitive adhesives can be synthesized in several ways, including solvent-borne, water-borne, and hot-melt methods. Tack is the initial adhesion under slight pressure and short dwell time and depends on the adhesive's ability to wet the contact surface. Peel strength is the force required to remove the PSA from the contact surface. The peel adhesion depends on many factors, including the tack, bonding history (e.g. force, dwell time), and adhesive composition. Shear strength is a measure of the adhesive's resistance to continuous stress. The shear strength is influenced by several parameters, including internal adhesion, cross-linking, and viscoelastic properties of the adhesive. Permanent adhesives are generally resistant to debonding and possess very high peel and shear strength. Pressure-sensitive adhesives may include natural rubber, synthetic rubber (e.g., styrene-butadiene and styrene-ethylene copolymers), polyvinyl ether, polyurethane, acrylic, silicones, and ethylene-vinyl acetate copolymers. A copolymer's adhesive properties can be altered by varying the composition (via monomer components) changing the glass transition temperature (Tg) or degree of cross-linking. In general, a copolymer with a lower Tg is less rigid and a copolymer with a higher Tg is more rigid. The tack of PSAs can be altered by the addition of components to alter the viscosity or mechanical properties. Pressure sensitive adhesives are further described in Czech et al., “Pressure-Sensitive Adhesives for Medical Applications,” in Wide Spectra of Quality Control, Dr. Isin Akyar (Ed., published by InTech), Chapter 17 (2011), which is hereby incorporated by reference in its entirety.

A system, apparatus, method, or kit may contain or be used to deliver one or more useful therapeutic agents. For example, the hollow needles of an apparatus as described herein may be configured to administer one or more therapeutic agents to the skin. In some embodiments, hollow needles of an apparatus as described herein may be used to create direct channels or holes to the local blood supply and local perfusion by removing cored tissue portions. In some embodiments, direct channels or holes may be used to deliver one or more useful therapeutic agents. Depending on the size (e.g., diameter and/or active length) of hollow needles, holes having different diameters and/or penetration depths may be created. For example, hollow needles having a large diameter (e.g., 18 gauge) and/or a long active length may be used to create large and/or deep holes that may be used as delivery channels to deliver a large volume dose of therapeutic agents. In some embodiments, holes may be plugged. In some embodiments, holes may be covered with a dressing (e.g., a compressive or occlusive dressing) and/or a closure (e.g., bandage, hemostats, sutures, or adhesives) to prevent the delivered therapeutic agents from leaking out of the skin and/or to maintain moisture of the treated skin area. Delivery of useful therapeutic agents through the holes created by the hollow needles of the apparatus may provide precise control of dosing of the therapeutic agents.

Examples of therapeutic agents that may be delivered using the technologies described herein include one or more growth factors (e.g., vascular endothelial growth factor (VEGF), platelet-derived growth factor (PDGF), transforming growth factor beta (TGF-β), fibroblast growth factor (FGF), epidermal growth factor (EGF), and keratinocyte growth factor); one or more stem cells (e.g., adipose tissue-derived stem cells and/or bone marrow-derived mesenchymal stem cells); one or more skin whitening agents (e.g., hydroquinone); one or more vitamin A derivatives (e.g., tretinoin), one or more analgesics (e.g., paracetamol/acetaminophen, aspirin, a non-steroidal antiinflammatory drug, as described herein, a cyclooxygenase-2-specific inhibitor, as described herein, dextropropoxyphene, co-codamol, an opioid (e.g., morphine, codeine, oxycodone, hydrocodone, dihydromorphine, pethidine, buprenorphine, tramadol, or methadone), fentanyl, procaine, lidocaine, tetracaine, dibucaine, benzocaine, p-butylaminobenzoic acid 2-(diethylamino) ethyl ester HCl, mepivacaine, piperocaine, dyclonine, or venlafaxine); one or more antibiotics (e.g., cephalosporin, bactitracin, polymyxin B sulfate, neomycin, bismuth tribromophenate, or polysporin); one or more antifungals (e.g., nystatin); one or more antiinflammatory agents (e.g., a non-steroidal antiinflammatory drug (NSAID, e.g., ibuprofen, ketoprofen, flurbiprofen, piroxicam, indomethacin, diclofenac, sulindac, naproxen, aspirin, ketorolac, or tacrolimus), a cyclooxygenase-2-specific inhibitor (COX-2 inhibitor, e.g., rofecoxib (Vioxx®), etoricoxib, and celecoxib (Celebrex®)), a glucocorticoid agent, a specific cytokine directed at T lymphocyte function), a steroid (e.g., a corticosteroid, such as a glucocorticoid (e.g., aldosterone, beclometasone, betamethasone, cortisone, deoxycorticosterone acetate, dexamethasone, fludrocortisone acetate, hydrocortisone, methylprednisolone, prednisone, prednisolone, or triamcinolone) or a mineralocorticoid agent (e.g., aldosterone, corticosterone, or deoxycorticosterone)), or an immune selective antiinflammatory derivative (e.g., phenylalanine-glutamine-glycine (FEG) and its D-isomeric form (feG))); one or more antimicrobials (e.g., chlorhexidine gluconate, iodine (e.g., tincture of iodine, povidone-iodine, or Lugol's iodine), or silver, such as silver nitrate (e.g., as a 0.5% solution), silver sulfadiazine (e.g., as a cream), or Ag⁺ in one or more useful carriers (e.g., an alginate, such as Acticoat® including nanocrystalline silver coating in high density polyethylene, available from Smith & Nephew, London, U.K., or Silvercel® including a mixture of alginate, carboxymethylcellulose, and silver coated nylon fibers, available from Systagenix, Gatwick, U.K.; a foam (e.g., Contreet® Foam including a soft hydrophilic polyurethane foam and silver, available from Coloplast A/S, Humlebck, Denmark); a hydrocolloid (e.g., Aquacel® Ag including ionic silver and a hydrocolloid, available from Conva Tec Inc., Skillman, N.J.); or a hydrogel (e.g., Silvasorb® including ionic silver, available from Medline Industries Inc., Mansfield, Mass.)); one or more antiseptics (e.g., an alcohol, such as ethanol (e.g., 60-90%), 1-propanol (e.g., 60-70%), as well as mixtures of 2-propanol/isopropanol; boric acid; calcium hypochlorite; hydrogen peroxide; manuka honey and/or methylglyoxal; a phenol (carbolic acid) compound, e.g., sodium 3,5-dibromo-4-hydroxybenzene sulfonate, trichlorophenylmethyl iodosalicyl, or triclosan; a polyhexanide compound, e.g., polyhexamethylene biguanide (PHMB); a quaternary ammonium compound, such as benzalkonium chloride (BAC), benzethonium chloride (BZT), cetyl trimethylammonium bromide (CTMB), cetylpyridinium chloride (CPC), chlorhexidine (e.g., chlorhexidine gluconate), or octenidine (e.g., octenidine dihydrochloride); sodium bicarbonate; sodium chloride; sodium hypochlorite (e.g., optionally in combination with boric acid in Dakin's solution); or a triarylmethane dye (e.g., Brilliant Green)); one or more antiproliferative agents (e.g., sirolimus, tacrolimus, zotarolimus, biolimus, or paclitaxel); one or more emollients; one or more hemostatic agents (e.g., collagen, such as microfibrillar collagen, chitosan, calcium-loaded zeolite, cellulose, anhydrous aluminum sulfate, silver nitrate, potassium alum, titanium oxide, fibrinogen, epinephrine, calcium alginate, poly-N-acetyl glucosamine, thrombin, coagulation factor(s) (e.g., II, V, VII, VIII, IX, X, XI, XIII, or Von Willebrand factor, as well as activated forms thereof), a procoagulant (e.g., propyl gallate), an anti-fibrinolytic agent (e.g., epsilon aminocaproic acid or tranexamic acid), and the like); one or more procoagulative agents (e.g., any hemostatic agent described herein, desmopressin, coagulation factor(s) (e.g., II, V, VII, VIII, IX, X, XI, XIII, or Von Willebrand factor, as well as activated forms thereof), procoagulants (e.g., propyl gallate), antifibrinolytics (e.g., epsilon aminocaproic acid), and the like); one or more anticoagulative agents (e.g., heparin or derivatives thereof, such as low molecular weight heparin, fondaparinux, or idraparinux; an anti-platelet agent, such as aspirin, dipyridamole, ticlopidine, clopidogrel, or prasugrel; a factor Xa inhibitor, such as a direct factor Xa inhibitor, e.g., apixaban or rivaroxaban; a thrombin inhibitor, such as a direct thrombin inhibitor, e.g., argatroban, bivalirudin, dabigatran, hirudin, lepirudin, or ximelagatran; or a coumarin derivative or vitamin K antagonist, such as warfarin (coumadin), acenocoumarol, atromentin, phenindione, or phenprocoumon); one or more immune modulators, including corticosteroids and non-steroidal immune modulators (e.g., NSAIDS, such as any described herein); one or more proteins; and/or one or more vitamins (e.g., vitamin A, C, and/or E). One or more of botulinum toxin, fat (e.g. autologous), hyaluronic acid, a collagen-based filler, or other filler may also be administered to the skin. Platelet rich plasma may also be administered to the skin. One or more therapeutic agents described herein may be formulated as a depot preparation. In general, depot preparations are typically longer acting than non-depot preparations. In some embodiments, depot preparations are prepared using suitable polymeric or hydrophobic materials (for example an emulsion in an acceptable oil) or ion exchange resins, or as sparingly soluble derivatives, for example, as a sparingly soluble salt.

In some embodiments, a therapeutic agent may include anticoagulative and/or procoagulative agents. For instance, by controlling the extent of bleeding and/or clotting in treated skin regions, a skin tightening effect may be more effectively controlled. Thus, in some embodiments, the methods and devices herein include or can be used to administer one or more anticoagulative agents, one or more procoagulative agents, one or more hemostatic agents, one or more fillers, or combinations thereof. In particular embodiments, the therapeutic agent controls the extent of bleeding and/or clotting in the treated skin region, including the use one or more anticoagulative agents (e.g., to inhibit clot formation prior to skin healing or slit/hole closure) and/or one or more hemostatic or procoagulative agents.

Components of different embodiments described in this specification may be combined to form other embodiments not specifically set forth in this specification. Components may be left out of the systems, apparatuses, etc. described in this specification without adversely affecting their operation. In addition, the logic flows shown in, or implied by, the figures do not require the particular order shown, or sequential order, to achieve desirable results. Various separate components may be combined into one or more individual components to perform the functions described here.

### EXAMPLE EMBODIMENTS

Embodiment 1: An apparatus for producing a cosmetic effect in skin tissue, the apparatus comprising:

(i) a needle hub comprising at least one hollow needle having a distal end for contacting skin and configured to remove a portion of the skin tissue (e.g., a microcore) when the hollow needle is inserted into and withdrawn from the skin tissue;

(ii) a translation and/or actuation mechanism connected to the needle hub to translate and/or actuate the needle hub in one or more directions relative to a surface of the skin tissue; and

(iii) a spacer to stabilize and/or maintain a constant position of the apparatus relative to the surface of the skin tissue.

Embodiment 2: The apparatus of Embodiment 1, comprising a hand piece shell at least partially enclosing the translation and/or actuation mechanism.

Embodiment 3: The apparatus of Embodiment 1 or Embodiment 2, wherein the spacer is attached to the hand piece shell.

Embodiment 4: The apparatus of any of Embodiments 1-3, wherein the needle hub comprises a single hollow needle.

Embodiment 5: The apparatus of any of Embodiments 1-4, wherein the needle hub comprises three hollow needles arranged in a row.

Embodiment 6: The apparatus of any of Embodiments 1-5, wherein the needle hub comprises a two dimensional array of needles (e.g., a two-by-two, three-by-two, or three-by-three array).

Embodiment 7: The apparatus of any of Embodiments 1-6, wherein the needle hub comprises a first lumen having a first end and a second end, wherein the first lumen comprises a lumen of the at least one hollow needle and wherein the first end of the first lumen is at the distal end of the hollow needle.

Embodiment 8: The apparatus of any of Embodiments 1-7, wherein the needle hub comprises a second lumen having a wall, a first end, and a second end, wherein the first end of the second lumen is or comprises a fluid intake nozzle.

Embodiment 9: The apparatus of any of Embodiments 1-8, wherein the first lumen is connected to the second lumen such that the second end of the first lumen forms an opening in the wall of the second lumen.

Embodiment 10: The apparatus of any of Embodiments 1-9, wherein each of the first lumen and the second lumen are substantially straight, and wherein the first lumen is substantially perpendicular to the second lumen forming a T-junction.

Embodiment 11: The apparatus of any of Embodiments 1-10, wherein the fluid intake nozzle is a convergent nozzle.

Embodiment 12: The apparatus of any of Embodiments 1-11, wherein the second end of the second lumen is connected to a fluid conduit such that when low pressure or vacuum is applied to the conduit, low pressure or vacuum is induced in the first lumen and the second lumen, such that fluid is drawn into and through the second lumen through the first end of the second lumen, thereby clearing skin tissue from the first lumen.

Embodiment 13: The apparatus of any of Embodiments 1-12, wherein the translation and/or actuation mechanism comprises an actuator to displace the needle hub along a z-axis in a direction substantially perpendicular to a surface of the skin tissue and substantially parallel to a longitudinal axis of the at least one hollow needle.

Embodiment 14: The apparatus of any of Embodiments 1-13, wherein the actuator is or comprises a voice coil.

Embodiment 15: The apparatus of any of Embodiments 1-14, comprising a sensing device for detecting a position of the needle hub along the z-axis.

Embodiment 16: The apparatus of any of Embodiments 1-15, wherein the translation and/or actuation mechanism comprises an x/y-stage to translate the needle hub in one or more directions parallel to the surface of the skin.

Embodiment 17: The apparatus of any of Embodiments 1-16, wherein the translation and/or actuation mechanism comprises a rotary stage to rotate the needle hub around the z-axis.

Embodiment 18: The apparatus of any of Embodiments 1-17, wherein the spacer comprises a device to contact a surface of the skin tissue, and to (a) to maintain a distance and/or position between the apparatus and the skin tissue and/or (b) maintain or increase tension in the skin tissue during treatment compared to the skin tissue not being treated and/or contacted by an apparatus.

Embodiment 19: The apparatus of any of Embodiments 1-18, wherein the spacer comprises a frame to contact the surface of the skin tissue, wherein the frame comprises a base, an inner wall, and an outer wall, wherein the base, inner wall, and outer wall form an open channel.

Embodiment 20: The apparatus of any of Embodiments 1-19, wherein the channel is configured such that when the frame is placed on the surface of the skin, the surface of the skin, the base, the inner wall, and outer wall form a frame lumen.

Embodiment 21: The apparatus of any of Embodiments 1-20, wherein the frame is connected to a fluid conduit such that when low pressure or vacuum is applied to the conduit, low pressure or vacuum is established in the frame lumen, thereby drawing skin tissue toward and/or into the channel.

Embodiment 22: The apparatus of any of Embodiments 1-21, wherein the base comprises one or more protrusions.

Embodiment 23: The apparatus of any of Embodiments 1-22, wherein the frame is contoured (e.g., wherein the frame is concave).

Embodiment 24: The apparatus of any of Embodiments 1-23, wherein the spacer comprises a switch connected to a sensor to detect a position of the apparatus relative to tissue underlying the skin, wherein

(a) when the frame is placed on the surface of the skin and a low pressure or vacuum is applied to the frame, the switch is in a “no-go” position, and

(b) when the frame, while the frame is in contact with the surface of the skin after a low pressure or vacuum is applied to the frame, and after the frame is moved in a direction that is substantially perpendicular to and away from the surface of the skin, the switch is in a “go” position;

wherein, when the switch is in the no-go position, the needle hub is prevented from moving along a z-axis in a direction substantially perpendicular to a surface of the skin tissue and substantially parallel to a longitudinal axis of the at least one hollow needle; and

wherein, when the switch is in the go position, the needle hub is moveable along the z-axis.

Embodiment 25: The apparatus of any of Embodiments 1-24, wherein the sensor is or comprises a pushrod.

Embodiment 26: A system comprising the apparatus of any of Embodiments 1-25, the system comprising a removal system for removing one or more tissue portions from the apparatus.

Embodiment 27: The system of Embodiment 26, wherein the removal system comprises a low pressure source (e.g., a vacuum pump).

Embodiment 28: The system of any of Embodiments 26-27, wherein the low pressure source is connected to the needle hub comprising the at least one hollow needle via a first conduit to provide suction in the at least one hollow needle.

Embodiment 29: The system of any of Embodiments 26-28, wherein the low pressure source is connected to the spacer via a second conduit to provide suction in the spacer.

Embodiment 30: The apparatus of any of Embodiments 1-24, wherein the at least one hollow needle comprises at least a first prong provided at a distal end of the hollow needle for contacting skin, wherein an angle between a lateral side of the first prong and a longitudinal axis of the hollow needle is at least about 20 degrees.

Embodiment 31: The apparatus of any of Embodiments 1-24, wherein the at least one hollow needle comprises a second prong at the distal end of the hollow needle.

Embodiment 32: The apparatus of any of Embodiments 1-24, wherein the first prong and/or the second prong comprises a flat tip.

Embodiment 33: The apparatus of any of Embodiments 1-24, wherein the first prong and/or the second prong comprises an edge.

Embodiment 34: The apparatus of any of Embodiments 1-24, wherein an inner diameter of the at least one hollow needle is between about 0.14 mm and 0.84 mm.

Embodiment 35: The apparatus of any of Embodiments 1-24, wherein an inner diameter of the at least one hollow needle is between about 0.24 mm and 0.40 mm.

Embodiment 36: The apparatus of any of Embodiments 1-24, wherein the at least one hollow needle is configured to extend (i) into the dermal layer, (ii) through the entire dermal layer to the junction of the dermal layer and the subcutaneous fat layer, or (iii) into the subcutaneous fat layer.

Embodiment 37: An apparatus comprising a hollow needle and a pushrod moveably disposed therein.

Elements of different embodiments described herein may be combined to form other embodiments not specifically set forth above. Elements may be left out of the technologies, systems, apparatuses. computer programs, user interfaces, etc. described herein without adversely affecting their operation or the operation of the technologies in general. Furthermore, various separate elements may be combined into one or more individual elements to perform the functions described herein.

The foregoing description of various embodiments has been presented for purposes of illustration and description. The foregoing description is not intended to limit the claims to the embodiment disclosed herein.

What is claimed is:

1. An apparatus for producing a cosmetic effect in skin tissue, the apparatus comprising:
(i) a needle hub comprising at least one hollow needle having a distal end for contacting skin and configured to remove a portion of the skin tissue (e.g., a microcore) when the hollow needle is inserted into and withdrawn from the skin tissue;
(ii) a translation and/or actuation mechanism connected to the needle hub to translate and/or actuate the needle hub in one or more directions relative to a surface of the skin tissue; and
(iii) a spacer to stabilize and/or maintain a constant position of the apparatus relative to the surface of the skin tissue.

2. The apparatus of claim 1, comprising a hand piece shell at least partially enclosing the translation and/or actuation mechanism.

3. The apparatus of claim 2, wherein the spacer is attached to the hand piece shell.

4. The apparatus of claim 1, wherein the needle hub comprises a single hollow needle.

5. The apparatus of claim 1, wherein the needle hub comprises three hollow needles arranged in a row.

6. The apparatus of claim 1, wherein the needle hub comprises a two dimensional array of needles (e.g., a two-by-two, three-by-two, or three-by-three array).

7. The apparatus of claim 1, wherein the needle hub comprises a first lumen having a first end and a second end, wherein the first lumen comprises a lumen of the at least one hollow needle and wherein the first end of the first lumen is at the distal end of the hollow needle.

8. The apparatus of claim 7, wherein the needle hub comprises a second lumen having a wall, a first end, and a second end, wherein the first end of the second lumen is or comprises a fluid intake nozzle.

9. The apparatus of claim 8, wherein the first lumen is connected to the second lumen such that the second end of the first lumen forms an opening in the wall of the second lumen.

10. The apparatus of claim 9, wherein each of the first lumen and the second lumen are substantially straight, and wherein the first lumen is substantially perpendicular to the second lumen forming a T-junction.

11. The apparatus of claim 7, wherein the fluid intake nozzle is a convergent nozzle.

12. The apparatus of claim 9, wherein the second end of the second lumen is connected to a fluid conduit such that when low pressure or vacuum is applied to the conduit, low pressure or vacuum is induced in the first lumen and the second lumen, such that fluid is drawn into and through the second lumen through the first end of the second lumen, thereby clearing skin tissue from the first lumen.

13. The apparatus of claim 1, wherein the translation and/or actuation mechanism comprises an actuator to displace the needle hub along a z-axis in a direction substantially perpendicular to a surface of the skin tissue and substantially parallel to a longitudinal axis of the at least one hollow needle.

14. The apparatus of claim 13, wherein the actuator is or comprises a voice coil.

15. The apparatus of claim 13, comprising a sensing device for detecting a position of the needle hub along the z-axis.

16. The apparatus of claim 1, wherein the translation and/or actuation mechanism comprises an x/y-stage to translate the needle hub in one or more directions parallel to the surface of the skin.

17. The apparatus of claim 1, wherein the translation and/or actuation mechanism comprises a rotary stage to rotate the needle hub around the z-axis.

18. The apparatus of claim 1, wherein the spacer comprises a device to contact a surface of the skin tissue, and to (a) to maintain a distance and/or position between the apparatus and the skin tissue and/or (b) maintain or increase tension in the skin tissue during treatment compared to the skin tissue not being treated and/or contacted by an apparatus.

19. The apparatus of claim 1, wherein the spacer comprises a frame to contact the surface of the skin tissue, wherein the frame comprises a base, an inner wall, and an outer wall, wherein the base, inner wall, and outer wall form an open channel.

20. The apparatus of claim 19, wherein the channel is configured such that when the frame is placed on the surface of the skin, the surface of the skin, the base, the inner wall, and outer wall form a frame lumen.

21. The apparatus of claim 20, wherein the frame is connected to a fluid conduit such that when low pressure or vacuum is applied to the conduit, low pressure or vacuum is established in the frame lumen, thereby drawing skin tissue toward and/or into the channel.

22. The apparatus of claim 19, wherein the base comprises one or more protrusions.

23. The apparatus of claim 19, wherein the frame is contoured (e.g., wherein the frame is concave).

24. The apparatus of claim 19, wherein the spacer comprises a switch connected to a sensor to detect a position of the apparatus relative to tissue underlying the skin, wherein
(a) when the frame is placed on the surface of the skin and a low pressure or vacuum is applied to the frame, the switch is in a “no-go” position, and
(b) when the frame, while the frame is in contact with the surface of the skin after a low pressure or vacuum is applied to the frame, and after the frame is moved in a direction that is substantially perpendicular to and away from the surface of the skin, the switch is in a “go” position;
wherein, when the switch is in the no-go position, the needle hub is prevented from moving along a z-axis in a direction substantially perpendicular to a surface of the skin tissue and substantially parallel to a longitudinal axis of the at least one hollow needle; and
wherein, when the switch is in the go position, the needle hub is moveable along the z-axis.

25. The apparatus of claim 24, wherein the sensor is or comprises a pushrod.

26. A system comprising the apparatus of claim 1, the system comprising a removal system for removing one or more tissue portions from the apparatus.

27. The system of claim 26, wherein the removal system comprises a low pressure source (e.g., a vacuum pump).

28. The system of claim 27, wherein the low pressure source is connected to the needle hub comprising the at least one hollow needle via a first conduit to provide suction in the at least one hollow needle.

29. The system of claim 28, wherein the low pressure source is connected to the spacer via a second conduit to provide suction in the spacer.

30. The apparatus of claim 1, wherein the at least one hollow needle comprises at least a first prong provided at a distal end of the hollow needle for contacting skin, wherein an angle between a lateral side of the first prong and a longitudinal axis of the hollow needle is at least about 20 degrees.

31. The apparatus of claim 30, wherein the at least one hollow needle comprises a second prong at the distal end of the hollow needle.

32. The apparatus of claim 31, wherein the first prong and/or the second prong comprises a flat tip.

33. The apparatus of claim 31, wherein the first prong and/or the second prong comprises an edge.

34. The apparatus of claim 1, wherein an inner diameter of the at least one hollow needle is between about 0.14 mm and 0.84 mm.

35. The apparatus of claim 1, wherein an inner diameter of the at least one hollow needle is between about 0.24 mm and 0.40 mm.

36. The apparatus of claim 1, wherein the at least one hollow needle is configured to extend (i) into the dermal layer, (ii) through the entire dermal layer to the junction of the dermal layer and the subcutaneous fat layer, or (iii) into the subcutaneous fat layer.

37. An apparatus comprising a hollow needle and a pushrod moveably disposed therein.
